(19) United States
(12) Patent Application Publication
(10) Pub. No.: US 2025/0281491 A1
(43) Pub. Date: Sep. 11, 2025

(54) DUAL FUNCTIONING IMMUNE MODULATING COMPOUNDS, FORMULATIONS, AND USES THEREOF

(71) Applicant: THE REGENTS OF THE UNIVERSITY OF MICHIGAN, Ann Arbor, MI (US)

(72) Inventors: Duxin Sun, Ann Arbor, MI (US); Chengyi Li, Ann Arbor, MI (US); Wei Gao, Ann Arbor, MI (US); Mahamadou Djibo, Ann Arbor, MI (US); Mohamed Dit Mady Traore, Ann Arbor, MI (US); Zhongwei Liu, Ann Arbor, MI (US); Shuai Mao, Ann Arbor, MI (US)

(21) Appl. No.: 18/858,987

(22) PCT Filed: Apr. 25, 2023
(86) PCT No.: PCT/US2023/019804

§ 371 Date: Oct. 22, 2024

Related U.S. Application Data
(60) Provisional application No. 63/334,441, filed on Apr. 25, 2022.

(51) Int. Cl. A61K 31/519; A61K 9/51; A61K 31/405; A61K 39/00; A61K 45/06; C07D 409/12; C07D 487/04

(52) U.S. Cl. CPC A61K 31/519; A61K 9/5169; A61K 31/405; A61K 45/06; C07D 409/12; C07D 487/04; A61K 2039/53; A61K 2039/585

(57) ABSTRACT

The present disclosure provides dual functioning compounds, compositions, formulations, and methods for inducing or modulating an immune or inflammatory response and treating diseases or disorders (e.g., cancer, autoimmune diseases, inflammatory diseases, and infectious diseases) with the compounds or compositions thereof. In particular disclosed herein are dual functioning compounds comprising a stimulator of interferon (IFN) genes (STING) agonist moiety and a second active moiety selected from an indoleamine 2,3-dioxygenase (IDO) inhibitor and phosphatidylinositol 3-kinase (PI3K) inhibitor, and compositions and formulations thereof.

### CROSS-REFERENCE TO RELATED APPLICATIONS

This application claims the benefit of U.S. Provisional Application No. 63/334,441, filed Apr. 25, 2022, the content of which is herein incorporated by reference in its entirety.

### FIELD

The present disclosure provides dual functioning compounds, compositions, formulations, and methods for inducing or modulating an immune or inflammatory response and treating diseases or disorders (e.g., cancer, autoimmune diseases, inflammatory diseases, and infectious diseases) with the compounds or compositions thereof.

### BACKGROUND

The innate immune agonist STING (Stimulator of Interferon Genes) binds its natural ligand 2′3′-cGAMP (cyclic guanosine-adenosine monophosphate) and then, by way of its signaling pathway, induces the expression of interferons, inflammatory factors, and autophagy genes. Microbial infection, tumor DNA, and self-damaging DNA are three factors that induce the activation of the cGAS-STING signaling pathway and associate STING with the etiology of cancer, and autoimmune, infectious, and inflammatory diseases. Many natural and synthetic STING agonists have entered clinical development, particularly for cancer treatment, with the first generation demonstrating safety but only modest systemic activity. As such, most STING agonists require local delivery, due to their poor stability, thereby limiting their utility.

### SUMMARY

In one aspect, disclosed herein is a compound of formula (I):

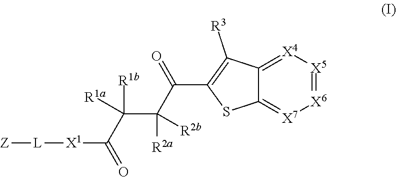

or a pharmaceutically acceptable salt thereof, wherein:
X¹ is selected from O, NR, S, and a bond;

R^{1a}, R^{1b}, R^{2a}, and R^{2b} are each independently selected from hydrogen, C₁-C₄ alkyl, C₁-C₄ alkoxy, C₃-C₆ cycloalkyl, halo-C₁-C₄-alkyl, amino-C₁-C₄-alkyl, hydroxy-C₁-C₄-alkyl, C₁-C₄-alkoxy-C₁-C₄-alkyl, halo, hydroxy, amino, C₁-C₄-alkylamino, di-C₁-C₄-alkylamino, and cyano; wherein R^{1a} and R^{1b}, or R^{1a} and R^{2a}, or R^{2a} and R^{2b}, are optionally taken together with the carbon atom(s) to which they are attached to form an optionally substituted 3- to 6-membered ring;
X⁴ is CR⁴ or N;
X⁵ is CR⁵ or N;
X⁶ is CR⁶ or N;
X⁷ is CR⁷ or N;
R³, R⁴, R⁵, R⁶, and R⁷ are each independently selected from hydrogen, C₁-C₆ alkyl, C₂-C₆ alkenyl, C₂-C₆ alkynyl, C₁-C₆ alkoxy, C₁-C₆ alkylthio, C₃-C₆ cycloalkyl, halo-C₁-C₆-alkyl, halo-C₁-C₆-alkoxy, amino-C₁-C₆-alkyl, hydroxy-C₁-C₆-alkyl, C₁-C₆-alkoxy-C₁-C₆-alkyl, halo, hydroxy, amino, C₁-C₄-alkylamino, di-C₁-C₄-alkylamino, cyano, —COOR^{x}, —CON(R^{y})₂, —SO₂R^{z}, an oligo- or poly-ethylene glycol chain, and a group-Y—R⁸; wherein R⁴ and R⁵, R⁵ and R⁶, or R⁶ and R⁷ are optionally taken together with the carbon atoms to which they are attached to form an optionally substituted 5- or 6-membered ring;
Y is selected from —C(O)—, —C(O)O—, —C(O)NR^{v}—, and —C(O)S—;
R⁸ is a lipid moiety having at least 8 carbon atoms;
R^{v}, R^{w}, R^{x}, R^{y}, and R^{z} are each independently selected from hydrogen, C₁-C₆ alkyl, C₂-C₆ alkenyl, C₂-C₆ alkynyl, C₃-C₆ cycloalkyl, and halo-C₁-C₆-alkyl;
L is a linker; and
Z is a moiety of formula (a) or formula (b):

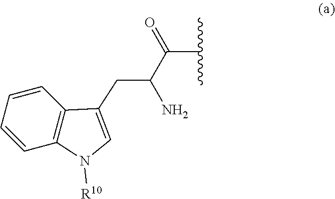

wherein R¹⁰ is hydrogen, or C₁-C₆ alkyl;

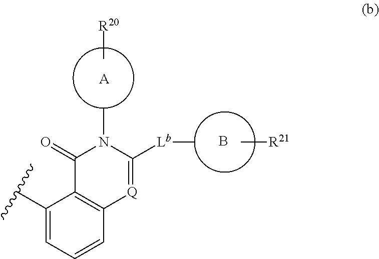

wherein:
Q is CH or N;
A is aryl or a 5- or 6-membered monocyclic heteroaryl having 1, 2, 3, or 4 heteroatoms independently selected from N, O, S, and P;
R²⁰ is selected from hydrogen, halo, C₁-C₄ alkyl, C₃-C₆ cycloalkyl, C₁-C₄ haloalkyl, —OR^{a1}, —N(R^{a2})(R^{a3}), —SO₂R^{a4}, —SO₂N(R^{a5})(R^{a6}), and —NHSO₂R^{a7}, wherein R^{a1}, R^{a2}, R^{a3}, R^{a4}, R^{a5}, R^{a6}, and R^{a7} are each independently selected from hydrogen, C₁-C₄ alkyl, and C₁-C₄ haloalkyl;
R²¹ is selected from hydrogen and a group-L²¹-E, wherein:
   L²¹ is a bond, C₁-C₂ alkylene, —CH═CH—, —C≡C—, —C(O)—, —O—, —NH—, —S—, —C(O)O—, —C(O)NH—, —C(O)S—, arylene, cycloalkylene, heteroarylene, or heterocyclylene, or wherein L²¹ comprises a combination of any two of such groups;
   E is a bicyclic heterocyclyl or bicyclic heteroaryl, each of which is optionally substituted with 1, 2, 3, 4, or 5 substituents independently selected from halo, C₁-C₄ alkyl, C₃-C₆ cycloalkyl, C₃-C₆-cycloalkyl-C₁₋₄-alkyl, C₁-C₄ haloalkyl, oxo, —OR^{b1}, —N(R^{b2})(R^{b3}), —SO₂R^{b4}, —SO₂N(R^{b5})(R^{b6}), and —NHSO₂R⁶⁷, wherein R^{b1}, R^{b2}, R^{b3}, R^{b4}, R^{b5}, R^{b6}, and R^{b7} are each independently selected from hydrogen, C₁-C₄ alkyl, and C₁-C₄ haloalkyl;
L^{b} is —(CR^{c1}R^{c2})ₘ-G^{b}-, wherein:
   R^{c1} and R^{c2} are independently selected from hydrogen and C₁-C₄ alkyl;
   m is 0, 1, or 2; and
   G^{b} is a bond, —NHC(O)—, —NH—, —O—, or —S—; and
B is a bicyclic heteroaryl or bicyclic heterocyclyl, each of which is optionally substituted with 1, 2, 3, 4, or 5 substituents independently selected from halo, C₁-C₄ alkyl, C₃-C₆ cycloalkyl, C₁-C₄ haloalkyl, optionally substituted aryl, —OR^{d1}, —N(R^{d2})(R^{d3}), —SO₂R^{d4}, —SO₂N(R^{d5})(R^{d6}), and —NHSO₂R^{d7}, wherein R^{d1}, R^{d2}, R^{d3}, R^{d4}, R^{d5}, R^{d6}, and R^{d7} are each independently selected from hydrogen, C₁-C₄ alkyl, and C₁-C₄ haloalkyl.

In some embodiments, X⁴ is CR⁴, X⁵ is CR⁵, X⁶ is CR⁶, and X⁷ is CR⁷.

In some embodiments, R⁴ is selected from hydrogen and halo. In some embodiments, R⁴ is hydrogen.

In some embodiments, R⁵ and R⁶ are each independently selected from C₁-C₆ alkyl, C₂-C₆ alkenyl, C₂-C₆ alkynyl, C₁-C₆ alkoxy, C₁-C₆ alkylthio, C₃-C₆ cycloalkyl, halo-C₁-C₆-alkyl, halo-C₁-C₆-alkoxy, amino-C₁-C₆-alkyl, hydroxy-C₁-C₆-alkyl, C₁-C₆-alkoxy-C₁-C₆-alkyl, halo, hydroxy, amino, C₁-C₄-alkylamino, di-C₁-C₄-alkylamino, cyano, —COOR^{x}, —CON(R^{y})₂, and —SO₂R^{z}. In some embodiments, R⁵ and R⁶ are each independently selected from C₁-C₄ alkyl, C₂-C₄ alkenyl, C₂-C₄ alkynyl, C₁-C₄ alkoxy, C₁-C₆ alkylthio, halo-C₁-C₄-alkyl, halo-C₁-C₄-alkoxy, hydroxy, halo, and C₁-C₄-alkylamino. In some embodiments, R⁵ and R⁶ are each independently selected from C₁-C₄ alkyl, C₁-C₄ alkoxy, and halo. In some embodiments, R⁵ and R⁶ are each independently selected from C₁-C₄ alkoxy.

In some embodiments, R⁷ is hydrogen.

In some embodiments, R^{1a}, R^{1b}, R^{2a}, and R^{2b} are each independently selected from hydrogen, C₁-C₄ alkyl, C₃-C₆ cycloalkyl, C₁-C₄-alkoxy-C₁-C₄-alkyl, and hydroxy. In some embodiments, R^{1a}, R^{1b}, R^{2a}, and R^{2b} are each independently selected from hydrogen and C₁-C₄ alkyl. In some embodiments, R^{1b}, R^{2a}, and R^{2b} are hydrogen, and R^{1a} is selected from hydrogen and C₁-C₄ alkyl. In some embodiments, R^{1a} and R^{2a} are hydrogen, and R^{1b} and R^{2b} together with the carbon atoms to which they are attached form a 3-membered ring.

In some embodiments, R³ is selected from hydrogen and halo. In some embodiments, R³ is hydrogen.

In some embodiments, the compound is a compound of formula (Ia):

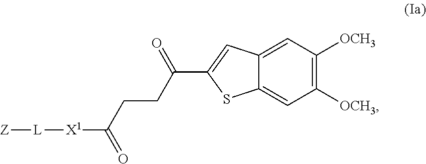

or a pharmaceutically acceptable salt thereof.

In some embodiments, Z is a moiety of formula (a). In some embodiments, R¹⁰ is methyl.

In some embodiments, Z is a moiety of formula (b). In some embodiments, the moiety of formula (b) is a moiety of formula (bi):

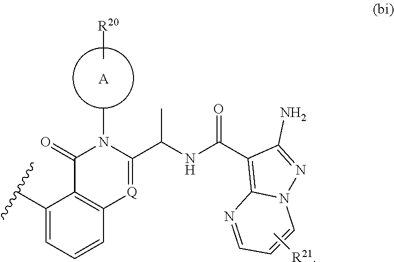

In some embodiments, A is phenyl. In some embodiments, R²⁰ is hydrogen.

In some embodiments, Q is CH. In some embodiments, R²¹ is selected from hydrogen and a group of formula:

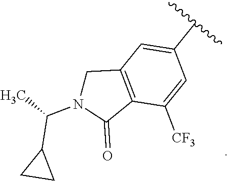 [SMILES: *c1cc2c(c(C(F)(F)F)c1)C(=O)N([C@@H](C)C1CC1)C2]

In some embodiments, the moiety of formula (b) is:

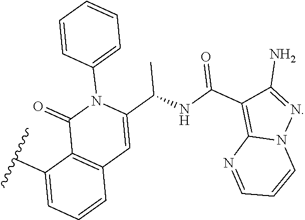 [SMILES: *c1cccc2cc([C@H](C)NC(=O)c3c(N)nn4cccnc34)n(-c3ccccc3)c(=O)c12]

In some embodiments, L comprises one or more groups independently selected from —C(R′)₂—, —CH═CH—, —C≡C—, —O—, —NR′—, —BR′—, —S—, —C(O)—, —C(NR′)—, —S(O)—, —S(O)₂—, arylene, heteroarylene, cycloalkylene, and heterocyclylene, wherein each R′ is independently selected from hydrogen, C₁-C₈₀ alkyl, C₂-C₈₀ alkenyl, C₂-C₈₀ alkynyl, aryl, arylalkyl, cycloalkyl, cycloalkylalkyl, heterocyclyl, heterocyclylalkyl, heteroaryl, and heteroarylalkyl, and wherein each alkyl, arylene, heteroarylene, cycloalkylene, and heterocyclylene is independently unsubstituted or substituted with 1, 2, or 3 substituents. In some embodiments, L comprises one or more groups independently selected from —C(R′)₂—, —C≡C—, —O—, —NH—, —C(O)—, and heteroarylene, wherein each R′ is independently selected from hydrogen, C₁-C₄₀ alkyl, phenyl, and —CH₂-heterocyclyl (e.g., wherein the heterocyclyl is a 6-membered heterocyclyl having 1 or 2 heteroatoms independently selected from N, O, and S), wherein the phenyl and the heterocyclyl are each independently unsubstituted or substituted with 1 or 2 substituents.

In some embodiments, the compound is selected from:

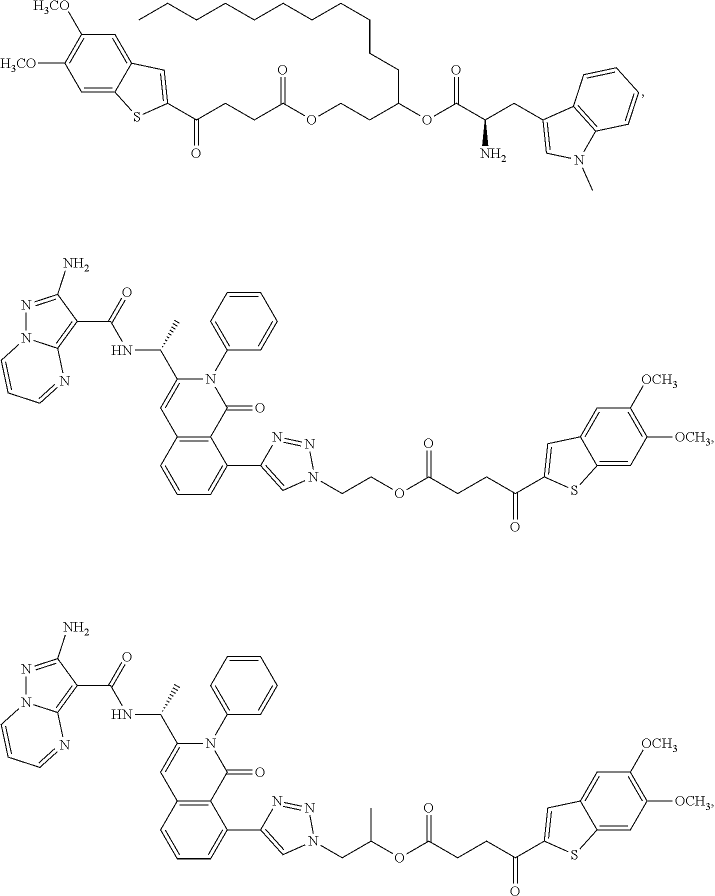 [SMILES: CCCCCCCCCCCC(CCOC(=O)CCC(=O)c1cc2cc(OC)c(OC)cc2s1)OC(=O)[C@H](N)Cc1cn(C)c2ccccc12 ; COc1cc2cc(C(=O)CCC(=O)OCCn3cc(-c4cccc5cc([C@@H](C)NC(=O)c6c(N)nn7cccnc67)n(-c6ccccc6)c(=O)c45)nn3)sc2cc1OC ; COc1cc2cc(C(=O)CCC(=O)OC(C)Cn3cc(-c4cccc5cc([C@@H](C)NC(=O)c6c(N)nn7cccnc67)n(-c6ccccc6)c(=O)c45)nn3)sc2cc1OC]

and pharmaceutically acceptable salts thereof.

In another aspect, disclosed herein is a pharmaceutical composition comprising an effective amount of a compound disclosed herein (e.g., a compound of formula (I)) or a pharmaceutically acceptable salt thereof, and a pharmaceutically acceptable carrier.

In some embodiments, the composition comprises albumin nanoparticles, liposomes, micelles, or lipid nanoparticles. In some embodiments, the composition further comprises an albumin nanoparticle. In some embodiments, the albumin is human serum albumin or albumin from animal species.

In some embodiments, the composition further comprises at least one additional therapeutic agent. In some embodiments, the at least one additional therapeutic agent comprises an immune modulator, a chemotherapeutic agent, a nucleic acid, a decongestant, a steroid, an analgesic, an antimicrobial agent, an immunotherapy, or a combination thereof. In some embodiments, the at least one additional therapeutic agent comprises an RNA selected from the group consisting of a small interfering RNA (siRNA), an asymmetrical interfering RNA (aiRNA), a microRNA (miRNA), a Dicer-substrate RNA (dsRNA), a small hairpin RNA (shRNA), a messenger RNA (mRNA), and mixtures thereof. In some embodiments, the at least one additional therapeutic agent is selected from a chemotherapeutic agent, an IDO inhibitor, a Stat3 inhibitor, a TLR agonist, PD-1 or PD-L1 antibody, and a PI3K inhibitor. In some embodiments, the composition further comprises one or more cell targeting epitopes. In some embodiments, the one or more cell targeting epitopes are covalently attached or directly conjugated to an albumin. In some embodiments, the cell targeting epitopes comprise an immune cell epitope. In some embodiments, the composition further comprises one or more epitopes from a microbiological agent.

In another aspect, disclosed herein is a vaccine comprising an effective amount of: a compound disclosed herein (e.g., a compound of formula (I), or a pharmaceutically acceptable salt thereof), or a pharmaceutical composition disclosed herein (e.g., a pharmaceutical composition comprising a compound of formula (I), or a pharmaceutically acceptable salt thereof); and an antigen or a nucleic acid encoding thereof.

In some embodiments, the antigen is a tumor antigen, a self-antigen, or an infectious disease derived antigen. In some embodiments, the nucleic acid is messenger RNA (mRNA).

In another aspect, disclosed herein is a method of treating or preventing a disease or disorder comprising administering an effective amount of a compound disclosed herein (e.g., a compound of formula (I), or a pharmaceutically acceptable salt thereof), or a pharmaceutical composition disclosed herein (e.g., a pharmaceutical composition comprising a compound of formula (I), or a pharmaceutically acceptable salt thereof), or a vaccine disclosed herein (e.g., a vaccine comprising a compound of formula (I), or a pharmaceutically acceptable salt thereof), to a subject in need thereof. In some embodiments, the disease or disorder comprises cancer, an autoimmune disease, an inflammatory disease, or an infectious disease. In some embodiments, the disease or disorder is cancer. In some embodiments, the subject has cancer, has had cancer, is predisposed to cancer, or has a family history of cancer. In some embodiments, the cancer comprises a solid tumor or hematological cancer. In some embodiments, the cancer is metastatic cancer. In some embodiments, the method suppresses or eliminates cancer metastasis, decreases tumor growth, prevents tumor recurrences, or any combination thereof. In some embodiments, the administering comprises an initial immunization and at least one subsequent immunization.

In another aspect, disclosed herein is a method of inducing or modulating an immune or inflammatory response in a subject comprising administering a compound disclosed herein (e.g., a compound of formula (I), or a pharmaceutically acceptable salt thereof), or a pharmaceutical composition disclosed herein (e.g., a pharmaceutical composition comprising a compound of formula (I), or a pharmaceutically acceptable salt thereof), or a vaccine disclosed herein (e.g., a vaccine comprising a compound of formula (I), or a pharmaceutically acceptable salt thereof), to a subject in need thereof. In some embodiments, the subject is human. In some embodiments, the method further comprises administering at least one additional therapeutic agent. In some embodiments, the at least one additional therapeutic agent comprises an immune modulator, a chemotherapeutic agent, a nucleic acid, a decongestant, a steroid, an analgesic, an antimicrobial agent, an immunotherapy, or a combination thereof.

In another aspect, disclosed herein is a use of compound disclosed herein (e.g., a compound of formula (I), or a pharmaceutically acceptable salt thereof), or a pharmaceutical composition disclosed herein (e.g., a pharmaceutical composition comprising a compound of formula (I), or a pharmaceutically acceptable salt thereof), in the manufacture of a medicament for the treatment or prevention a disease or disorder. In some embodiments, the disease or disorder comprises cancer, an autoimmune disease, an inflammatory disease, or an infectious disease.

Other aspects and embodiments of the disclosure will be apparent in light of the following detailed description.

### BRIEF DESCRIPTION OF THE DRAWINGS

FIG. 1 is a graph of STING activation for different concentrations of compounds as determined by incubating with THP-1 ISG Blue cells for 24 hours, mixing with ‘QUANTI-Blue’ solution and measuring OD 655.

FIG. 2 is a graph of STING activation for different concentrations of DMA01-166 as determined by incubating with THP-1 ISG Blue cells for 24 hours, mixing with ‘QUANTI-Blue’ solution and measuring OD 655.

FIG. 3 is a graph of STING activation for different concentrations of MSA-2 as determined by incubating with THP-1 ISG Blue cells for 24 hours, mixing with ‘QUANTI-Blue’ solution and measuring OD 655.

FIG. 4 are graphs of BMDC activation as measured based on the upregulation of CD80 and CD86 by flow cytometry for different concentrations of Compounds (cGAMP (10 μg/mL), IPI-549 (10 μM), MSA-2 (10 μM), DMA01-173 (10 μM), DMA01-166 (10 μM)) incubated with mouse bone marrow derived dendritic cells (BMDCs) for 24 hours.

FIG. 5 are graphs of BMDC activation as measured based on the upregulation of CD80 and CD86 by flow cytometry for different concentrations of DMA01-166 incubated with mouse bone marrow derived dendritic cells (BMDCs) for 24 hours.

FIG. 6 is a graph of mIFNβ concentrations as measured by ELISA from the supernatants of mouse bone marrow derived dendritic cells (BMDCs) incubated with different concentrations of Compounds (cGAMP (10 μg/mL), IPI-549 (10 μM), MSA-2 (10 μM), DMA01-173 (10 μM), DMA01-166 (10 μM)) for 24 hours.

FIG. 7 a graph of mIFNβ concentrations as measured by ELISA from the supernatants of mouse bone marrow derived dendritic cells (BMDCs) incubated with different concentrations of DMA01-166 for 24 hours.

FIG. 8 is a graph of TNFα concentrations as measured by ELISA from the supernatants of M2 polarized RAW264.7 cells incubated with different concentrations of compounds (cGAMP (10 μg/mL), ADU (10 μg/mL), IPI-549 (10 μM), MSA-2 (10 μM), DMA01-173 (10 μM), DMA01-170 (10 μM), DMA01-166 (10 μM)) for 2 days.

FIG. 9 is a graph of M1 macrophage ratio as determined by measuring CD80 and CD86 as markers for M1 macrophage by flow cytometry from M2 polarized RAW264.7 cells incubated with different concentrations of compounds (cGAMP (10 μg/mL), ADU (10 μg/mL), IPI-549 (10 μM), MSA-2 (10 μM), DMA01-173 (10 μM), DMA01-170 (10 M), DMA01-166 (10 μM)) for 2 days.

FIG. 10 is a graph of M1 macrophage ratio as determined by measuring CD80 and CD86 as markers for M1 macrophage by flow cytometry from M2 polarized RAW264.7 cells incubated with different concentrations of DMA01-166 for 2 days.

FIG. 11 are graphs of the inhibition of different isoforms of PI3K alpha, beta, gamma, delta by IPI-549, DMA01-148, 143, 132, 166, 170, and 173. Inhibition of PI3K activity by IPI-549, DMA01-148, 143, 132, 166, 170, 173 (0.1-10,000 nM) was measured using Kinase-Glo Max assay against purified enzymes PI3Kα, PI3K β, PI3Kγ, and PI3Kδ. The IC50 of compounds to inhibit different PI3K isoforms were calculated using Prism 8.

FIG. 12 is the size distribution figure (left) and average diameter and PDI changes (middle) and TEM image (right) of an exemplary albumin nanoformulation of DMA01-166.

FIG. 13 is a graph of the in vivo efficacy on KPC transgenic pancreatic cancer model of an exemplary albumin nanoformulations of: DMA01-166 (D166) and DMA01-166 plus PD-1 antibody (D166+PD-1) with and without paclitaxel (Nano-P).

### DETAILED DESCRIPTION

Described herein are dual functioning compounds comprising a stimulator of interferon (IFN) genes (STING) agonist moiety and a second active moiety selected from an indoleamine 2,3-dioxygenase (IDO) inhibitor and phosphatidylinositol 3-kinase (PI3K) inhibitor, and compositions and formulations thereof. One portion of the disclosed molecules acts as a STING agonist to target the cGAS-STING pathway, which is a cytosolic DNA-sensing pathway that drives activation of Type I IFN and other inflammatory cytokines. The other moiety of the dual functioning compounds acts as either a PI3K inhibitor or an IDO inhibitor, such that the compounds stimulate the immune response.

Section headings as used in this section and the entire disclosure herein are merely for organizational purposes and are not intended to be limiting.

### 1. DEFINITIONS

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular. All publications, patent applications, patents and other references mentioned herein are incorporated by reference in their entirety.

As used herein, “treat,” “treating,” and the like means a slowing, stopping, or reversing of progression of a disease or disorder when provided a compound or composition described herein to an appropriate control subject. The term also means a reversing of the progression of such a disease or disorder to a point of eliminating or greatly reducing the symptoms. As such, “treating” means an application or administration of the compositions described herein to a subject, where the subject has a disease or a symptom of a disease, where the purpose is to cure, heal, alleviate, relieve, alter, remedy, ameliorate, improve, or affect the disease or symptoms of the disease.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., humans and non-humans) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment, the mammal is a human.

As used herein, the terms “providing,” “administering,” and “introducing,” are used interchangeably herein and refer to the placement of the compounds or compositions of the disclosure into a subject by a method or route which results in at least partial localization of the compounds or composition to a desired site. The compounds or compositions can be administered by any appropriate route which results in delivery to a desired location in the subject.

The term “vaccine,” as used herein, refers to any pharmaceutical composition containing at least one antigenic or immunogenic peptide or other immunogen or at least one nucleic acid encoding at least one antigenic or immunogenic peptide or other immunogen, which can be used to prevent or treat a disease or condition in a subject.

The term “immunization,” as used herein, refers to a process that increases an organisms' reaction to antigen and therefore improves its ability to resist or overcome infection.

“Polynucleotide” or “oligonucleotide” or “nucleic acid,” as used herein, means at least two nucleotides covalently linked together. The polynucleotide may be DNA, both genomic and cDNA, RNA, or a hybrid, where the polynucleotide may contain combinations of deoxyribo- and ribo-nucleotides, and combinations of bases including uracil, adenine, thymine, cytosine, guanine, inosine, xanthine hypoxanthine, isocytosine and isoguanine. The nucleic acid, whether DNA or RNA may comprise non-natural nucleotides, modified nucleotides, and/or non-nucleotide building blocks that can exhibit the same function as natural nucleotides (e.g., “nucleotide analogs”). Nucleic acids may be obtained by chemical synthesis methods or by recombinant methods. Polynucleotides may be single- or double-stranded or may contain portions of both double stranded and single stranded sequence. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof.

A “peptide” or “polypeptide” is a linked sequence of two or more amino acids linked by peptide bonds. Peptides and polypeptides include proteins such as binding proteins, receptors, and antibodies. The terms “polypeptide” and “protein,” are used interchangeably herein.

As used herein, “nucleic acid” or “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41 (14): 4503-4510 (2002)) and U.S. Pat. No. 5,034,506), locked nucleic acid (LNA; see Wahlestedt et al., Proc. Natl. Acad. Sci. U.S.A., 97:5633-5638 (2000)), cyclohexenyl nucleic acids (see Wang, J. Am. Chem. Soc., 122:8595-8602 (2000)), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Sorrell, Organic Chemistry, 2^{nd} edition, University Science Books, Sausalito, 2006; Smith, March's Advanced Organic Chemistry: Reactions, Mechanism, and Structure, 7^{th} Edition, John Wiley & Sons, Inc., New York, 2013; Larock, Comprehensive Organic Transformations, 3^{rd} Edition, John Wiley & Sons, Inc., New York, 2018; and Carruthers, Some Modern Methods of Organic Synthesis, 3rd Edition, Cambridge University Press, Cambridge, 1987; the entire contents of each of which are incorporated herein by reference.

The term “alkyl,” as used herein, means a straight or branched, saturated hydrocarbon chain. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, 4,4-dimethylpentan-2-yl, n-heptyl, n-octyl, n-nonyl, n-decyl, undecyl, dodecyl, tridecyl, tetradecyl, pentadecyl, hexadecyl, heptadecyl, octadecyl, nonadecyl and icosyl.

The term “alkenyl,” as used herein, means a straight or branched hydrocarbon chain containing at least one carbon-carbon double bond. The double bond(s) may be located at any positions with the hydrocarbon chain. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkynyl,” as used herein, means a straight or branched hydrocarbon chain containing at least one carbon-carbon triple bond. The triple bond(s) may be located at any positions with the hydrocarbon chain. Representative examples of alkynyl include, but are not limited to, ethynyl, propynyl, and butynyl.

The term “alkoxy,” as used herein, refers to an alkyl group, as defined herein, appended to the parent molecular moiety through an oxygen atom. Representative examples of alkoxy include, but are not limited to, methoxy, ethoxy, n-propoxy, isopropoxy, n-butoxy, and tert-butoxy.

The term “alkoxyalkyl,” as used herein, refers to an alkyl group, as defined herein, in which at least one hydrogen atom (e.g., one hydrogen atom) is replaced with an alkoxy group, as defined herein. Representative examples of alkoxyalkyl include, but are not limited to, methoxymethyl.

The term “alkylthio,” as used herein, refers to an alkyl group, as defined herein, appended to the parent molecular moiety through a sulfur atom. Representative examples of alkoxy include, but are not limited to, methylthio, ethylthio, n-propylthio, isopropylthio, n-butylthio, and tert-butylthio.

The term “amino,” as used herein, refers to an —NH₂ μgroup. The term “alkylamino,” as used herein, refers to a group —NHR, wherein R is an alkyl group as defined herein. The term “dialkylamino,” as used herein, refers to a group —NR₂, wherein each R is independently an alkyl group as defined herein.

The term “aminoalkyl,” as used herein, refers to an alkyl group, as defined herein, in which at least one hydrogen atom (e.g., one hydrogen atom) is replaced with an amino group.

The term “cycloalkyl,” as used herein, refers to a saturated carbocyclic ring system containing three to ten carbon atoms and zero heteroatoms. The cycloalkyl may be monocyclic, bicyclic, bridged, fused, or spirocyclic. Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, bicyclo[2.2.1]heptanyl, bicyclo[3.2.1]octanyl, and bicyclo[5.2.0]nonanyl.

The term “cyano,” as used herein, refers to a group of formula —CN.

The term “halogen” or “halo,” as used herein, means F, Cl, Br, or I.

The term “haloalkyl,” as used herein, means an alkyl group, as defined herein, in which at least one hydrogen atom (e.g., one, two, three, four, five, six, seven or eight hydrogen atoms) is replaced with a halogen. In some embodiments, each hydrogen atom of the alkyl group is replaced with a halogen. Representative examples of haloalkyl include, but are not limited to, fluoromethyl, difluoromethyl, trifluoromethyl, 2-fluoroethyl, 2,2,2-trifluoroethyl, and 3,3,3-trifluoropropyl.

The term “haloalkoxy,” as used herein, means a haloalkyl group, as defined herein, is appended to the parent molecular moiety through an oxygen atom. Representative examples of haloalkoxy include, but are not limited to, difluoromethoxy, trifluoromethoxy, and 2,2,2-trifluoroethoxy.

The term “heteroalkyl,” as used herein, means an alkyl group, as defined herein, in which one or more of the carbon atoms (and any associated hydrogen atoms) are each independently replaced with a heteroatom group such as —NH—, —O—, —S—, —S(O)—, —S(O)₂—, —O—P(O)(O⁻)O—, or the like. By way of example, 1, 2, 3, 4, 5, 6, or more carbon atoms may be independently replaced with the same or different heteroatom group. A heteroalkyl group can also include one or more carbonyl moieties (i.e., wherein a carbon atom of the alkyl group is oxidized to a —C(O)— group).

The term “heteroalkenyl,” as used herein, refers to an alkenyl group, as defined herein, in which one or more of the carbon atoms (and any associated hydrogen atoms) are each independently replaced with a heteroatom group such as —NH—, —O—, —S—, —S(O)—, —S(O)₂—, or the like. By way of example, 1, 2, 3, 4, 5, 6, or more carbon atoms may be independently replaced with the same or different heteroatom group. A heteroalkenyl group can also include one or more carbonyl moieties (i.e., wherein a carbon atom of the alkyl group is oxidized to a —C(O)— group).

The term “heteroalkynyl,” as used herein, refers to an alkynyl group, as defined herein, in which one or more of the carbon atoms (and any associated hydrogen atoms) are each independently replaced with a heteroatom group such as —NH—, —O—, —S—, —S(O)—, —S(O)₂—, or the like. By way of example, 1, 2, 3, 4, 5, 6, or more carbon atoms may be independently replaced with the same or different heteroatom group. A heteroalkynyl group can also include one or more carbonyl moieties (i.e., wherein a carbon atom of the alkyl group is oxidized to a —C(O)— group).

The term “hydroxy,” as used herein, refers to an —OH group.

The term “hydroxyalkyl,” as used herein, refers to an alkyl group, as defined herein, in which at least one hydrogen atom (e.g., one hydrogen atom) is replaced with a hydroxy group.

As used herein, the term “substituent” refers to a group substituted on an atom of the indicated group.

When a group or moiety can be substituted, the term “substituted” indicates that one or more (e.g., 1, 2, 3, 4, 5, or 6; in some embodiments 1, 2, or 3; and in other embodiments 1 or 2) hydrogen atoms on the group indicated in the expression using “substituted” can be replaced with a selection of recited indicated groups or with a suitable substituent group known to those of skill in the art (e.g., one or more of the groups recited below), provided that the designated atom's normal valence is not exceeded. Substituent groups include, but are not limited to, alkyl, alkenyl, alkynyl, alkoxy, acyl, amino, amido, amidino, aryl, azido, carbamoyl, carboxyl, carboxyl ester, cyano, cycloalkyl, cycloalkenyl, guanidino, halo, haloalkyl, haloalkoxy, heteroalkyl, heteroaryl, heterocyclyl, hydroxy, hydrazino, imino, oxo, nitro, phosphate, phosphonate, sulfonic acid, sulfonamido, thiol, thione, thioxo, or combinations thereof.

As used herein, in chemical structures the indication;

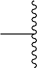

represents a point of attachment of one moiety to another moiety.

In some instances, the number of carbon atoms in a hydrocarbyl substituent (e.g., alkyl alkenyl) is indicated by the prefix “Cₓ-C_{y}”, wherein x is the minimum and y is the maximum number of carbon atoms in the substituent. Thus, for example, “C₁-C₃ alkyl” refers to an alkyl substituent containing from 1 to 3 carbon atoms.

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

Where substituent groups are specified by their conventional chemical formulae, written from left to right, they optionally encompass substituents resulting from writing the structure from right to left, e.g., —CH₂O— is intended to encompass —OCH₂—, and —C(O)NH— is intended to encompass —NHC(O)—.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. COMPOUNDS

In one aspect, disclosed is a compound of formula (I):

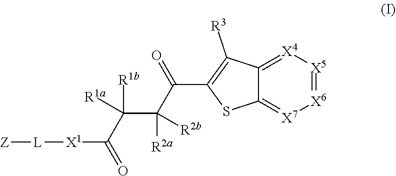

or a pharmaceutically acceptable salt thereof, wherein:
X¹ is selected from O, NR″, S, and a bond;
R^{1a}, R^{1b}, R^{2a}, and R^{2b} are each independently selected from hydrogen, C₁-C₄ alkyl, C₁-C₄ alkoxy, C₃-C₆ cycloalkyl, halo-C₁-C₄-alkyl, amino-C₁-C₄-alkyl, hydroxy-C₁-C₄-alkyl, C₁-C₄-alkoxy-C₁-C₄-alkyl, halo, hydroxy, amino, C₁-C₄-alkylamino, di-C₁-C₄-alkylamino, and cyano; wherein R^{1a} and R^{1b}, or R^{1a} and R^{2a}, or R^{2a} and R^{2b}, are optionally taken together with the carbon atom(s) to which they are attached to form an optionally substituted 3- to 6-membered ring;
X⁴ is CR⁴ or N;
X⁵ is CR⁵ or N;
X⁶ is CR⁶ or N;
X⁷ is CR⁷ or N;
R³, R⁴, R⁵, R⁶, and R⁷ are each independently selected from hydrogen, C₁-C₆ alkyl, C₂-C₆ alkenyl, C₂-C₆ alkynyl, C₁-C₆ alkoxy, C₁-C₆ alkylthio, C₃-C₆ cycloalkyl, halo-C₁-C₆-alkyl, halo-C₁-C₆-alkoxy, amino-C₁-C₆-alkyl, hydroxy-C₁-C₆-alkyl, C₁-C₆-alkoxy-C₁-C₆-alkyl, halo, hydroxy, amino, C₁-C₄-alkylamino, di-C₁-C₄-alkylamino, cyano, —COOR^{x}, —CON(R^{y})₂, —SO₂R^{z}, an oligo- or poly-ethylene glycol chain, and a group —Y—R⁸; wherein R⁴ and R⁵, R⁵ and R⁶, or R⁶ and R⁷ are optionally taken together with the carbon atoms to which they are attached to form an optionally substituted 5- or 6-membered ring;
Y is selected from —C(O)—, —C(O)O—, —C(O)NR^{v}—, and —C(O)S—;
R⁸ is a lipid moiety having at least 8 carbon atoms;
R^{v}, R^{w}, R^{x}, R^{y}, and R^{z} are each independently selected from hydrogen, C₁-C₆ alkyl, C₂-C₆ alkenyl, C₂-C₆ alkynyl, C₃-C₆ cycloalkyl, and halo-C₁-C₆-alkyl;
L is a linker; and
Z is a moiety of formula (a) or formula (b):

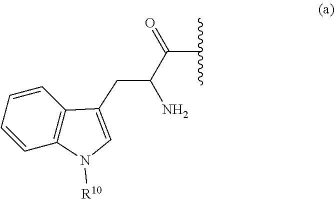

wherein R¹⁰ is hydrogen, or C₁-C₆ alkyl;

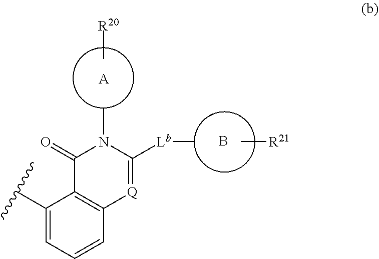

wherein:
Q is CH or N;
A is aryl or a 5- or 6-membered monocyclic heteroaryl having 1, 2, 3, or 4 heteroatoms independently selected from N, O, S, and P;
R²⁰ is selected from hydrogen, halo, C₁-C₄ alkyl, C₃-C₆ cycloalkyl, C₁-C₄ haloalkyl, —OR^{a1}, —N(R^{a2})(R^{a3}), —SO₂R^{a4}, —SO₂N(R^{a5})(R^{a6}), and —NHSO₂R^{a7}, wherein R^{a1}, R^{a2}, R^{a3}, R^{a4}, R^{a5}, R^{a6}, and R^{a7} are each independently selected from hydrogen, C₁-C₄ alkyl, and C₁-C₄ haloalkyl;
R²¹ is selected from hydrogen and a group -L²¹-E, wherein:
   L²¹ is a bond, C₁-C₂ alkylene, —CH═CH—, —C≡C—, —C(O)—, —O—, —NH—, —S—, —C(O)O—, —C(O)NH—, —C(O)S—, arylene, cycloalkylene, heteroarylene, or heterocyclylene, or wherein L²¹ comprises a combination of any two of such groups;
   E is a bicyclic heterocyclyl or bicyclic heteroaryl, each of which is optionally substituted with 1, 2, 3, 4, or 5 substituents independently selected from halo, C₁-C₄ alkyl, C₃-C₆ cycloalkyl, C₃-C₆-cycloalkyl-C₁₋₄-alkyl, C₁-C₄ haloalkyl, oxo, —OR^{b1}, —N(R^{b2})(R^{b3}), —SO₂R^{b4}, —SO₂N(R^{b5})(R^{b6}), and —NHSO₂R^{b7}, wherein R^{b1}, R^{b2}, R^{b3}, R^{b4}, R^{b5}, R^{b6}, and R^{b7} are each independently selected from hydrogen, C₁-C₄ alkyl, and C₁-C₄ haloalkyl;
L^{b} is —(CR^{c1}R^{c2})ₘ-G^{b}, wherein:
   R^{c1} and R^{c2} are independently selected from hydrogen and C₁-C₄ alkyl;
   m is 0, 1, or 2; and
   G^{b} is a bond, —NHC(O)—, —NH—, —O—, or —S—; and
B is a bicyclic heteroaryl or bicyclic heterocyclyl, each of which is optionally substituted with 1, 2, 3, 4, or 5 substituents independently selected from halo, C₁-C₄ alkyl, C₃-C₆ cycloalkyl, C₁-C₄ haloalkyl, optionally substituted aryl, —OR^{d1}, —N(R^{d2})(R^{d3}), —SO₂R^{d4}, —SO₂N(R^{d5})(R^{d6}), and —NHSO₂R^{d7}, wherein R^{d1}, R^{d2}, R^{d3}, R^{d4}, R^{d5}, R^{d6}, and R^{d7} are each independently selected from hydrogen, C₁-C₄ alkyl, and C₁-C₄ haloalkyl.

In some embodiments, R^{1a}, R^{1b}, R^{2a}, and R^{2b} are each independently selected from hydrogen, C₁-C₄ alkyl, C₃-C₆ cycloalkyl, C₁-C₄-alkoxy-C₁-C₄-alkyl, and hydroxy. In some embodiments, R^{1a}, R^{1b}, R^{2a}, and R^{2b} are each independently selected from hydrogen, methyl, ethyl, n-propyl, isopropyl, cyclopropyl, methoxymethyl, and hydroxy. In some embodiments, R^{1a}, R^{1b}, R^{2a}, and R^{2b} are each independently selected from hydrogen and C₁-C₄ alkyl. In some embodiments, R^{1a}, R^{1b}, R^{2a}, and R^{2b} are each independently selected from hydrogen and methyl. In some embodiments, one of R^{1a}, R^{1b}, R^{2a}, and R^{2b} is selected from C₁-C₄ alkyl, C₃-C₆ cycloalkyl, C₁-C₄-alkoxy-C₁-C₄-alkyl, and hydroxy, and the remaining three are hydrogen. In some embodiments, one of R^{1a}, R^{1b}, R^{2a}, and R^{2b} is selected from methyl, ethyl, n-propyl, isopropyl, cyclopropyl, methoxymethyl, and hydroxy, and the remaining three are hydrogen. In some embodiments, R^{1a}, R^{2a}, and R^{2b} are hydrogen, and R^{1a} is selected from hydrogen and C₁-C₄ alkyl. In some embodiments, R^{1b}, R^{2a}, and R^{2b} are hydrogen, and R^{1a} is selected from hydrogen and methyl. In some embodiments, R^{1a}, R^{1b}, R^{2a}, and R^{2b} are each hydrogen.

In some embodiments, R^{1a} and R^{2a} are hydrogen, and R^{1b} and R^{2b} together with the carbon atoms to which they are attached form a 3-membered ring (e.g., a cyclopropyl ring).

In some embodiments, R³ is selected from hydrogen and halo (e.g., fluoro, chloro, or bromo). In some embodiments, R³ is fluoro. In some embodiments, R³ is hydrogen. In some embodiments, X⁴ is CR⁴, X⁵ is CR⁵, X⁶ is CR⁶, and X⁷ is CR⁷.

In some embodiments, R⁴ is selected from hydrogen and halo (e.g., fluoro, chloro, or bromo). In some embodiments, R⁴ is fluoro. In some embodiments, R⁴ is hydrogen.

In some embodiments, R⁵ and R⁶ are each independently selected from C₁-C₆ alkyl, C₂-C₆ alkenyl, C₁-C₆ alkynyl, C₁-C₆ alkoxy, C₁-C₆ alkylthio, C₃-C₆ cycloalkyl, halo-C₁-C₆-alkyl, halo-C₁-C₆-alkoxy, amino-C₁-C₆-alkyl, hydroxy-C₁-C₆-alkyl, C₁-C₆-alkoxy-C₁-C₆-alkyl, halo, hydroxy, amino, C₁-C₄-alkylamino, di-C₁-C₄-alkylamino, cyano, —COOR^{x}, —CON(R^{y})₂, and —SO₂R^{z}. In some embodiments, R⁵ and R⁶ are each independently selected from C₁-C₄ alkyl, C₂-C₄ alkenyl, C₂-C₄ alkynyl, C₁-C₄ alkoxy, C₁-C₆ alkylthio, halo-C₁-C₄-alkyl, halo-C₁-C₄-alkoxy, hydroxy, halo, and C₁-C₄-alkylamino. In some embodiments, R⁵ and R⁶ are each independently selected from methyl, ethyl, n-propyl, ethenyl, ethynyl, methoxy, ethoxy, methylthio, 2-fluoroethyl, difluoromethoxy, hydroxy, fluoro, chloro, bromo, and methylamino. In some embodiments, R⁵ and R⁶ are each independently selected from C₁-C₄ alkyl, C₁-C₄ alkoxy, and halo. In some embodiments, R⁵ and R⁶ are each independently selected from methyl, ethyl, n-propyl, methoxy, ethoxy, fluoro, chloro, and bromo. In some embodiments, R⁵ and R⁶ are each independently selected from C₁-C₄ alkoxy. In some embodiments, R⁵ and R⁶ are each methoxy.

In some embodiments, R⁷ is selected from hydrogen and halo (e.g., fluoro, chloro, or bromo). In some embodiments, R⁷ is fluoro. In some embodiments, R⁷ is hydrogen.

In some embodiments, X¹ is O or a bond. In some embodiments, X¹ is O. In some embodiments, X¹ is a bond. In some embodiments, X¹ is NR^{w}, wherein R^{w} is selected from hydrogen and C₁-C₆ alkyl. In some embodiments, X¹ is NH. In some embodiments, X¹ is NR^{w}, wherein R^{w} is methyl. In some embodiments, X¹ is S.

In some embodiments, the compound is a compound of formula (Ia):

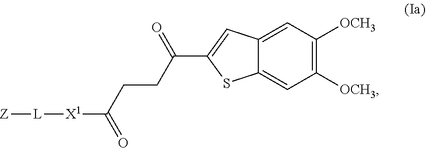

or a pharmaceutically acceptable salt thereof.

In some embodiments, Z is a moiety of formula (a). In some embodiments, R¹⁰ is C₁-C₆ alkyl. In some embodiments, R¹⁰ is methyl. In some embodiments, Z is a moiety of formula:

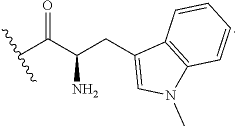 [SMILES: *C(=O)[C@H](N)Cc1cn(C)c2ccccc12]

In some embodiments, Z is a moiety of formula (b).

In some embodiments, A is phenyl or a monocyclic heteroaryl having 1, 2, 3, or 4 heteroatoms independently selected from N, O, S, and P. In some embodiments, A is phenyl or a monocyclic heteroaryl having 1, 2, 3, or 4 heteroatoms independently selected from N, O, and S. In some embodiments, A is phenyl or a monocyclic heteroaryl having one heteroatom selected from N, O, and S. In some embodiments, A is selected from phenyl, pyridyl, furan, and thiophene. In some embodiments, A is phenyl. In some embodiments, R²⁰ is hydrogen. In some embodiments, A is phenyl and R²⁰ is hydrogen.

In some embodiments, B is a nine-membered bicyclic heteroaryl or a nine-membered bicyclic heterocyclyl having 1, 2, 3, or 4 heteroatoms independently selected from N, O, S, and P. In some embodiments, B is a nine-membered bicyclic heteroaryl or a nine-membered bicyclic heterocyclyl having 1, 2, 3, or 4 heteroatoms independently selected from N, O, and S. In some embodiments, B is a nine-membered bicyclic heteroaryl or a nine-membered bicyclic heterocyclyl having 1, 2, 3, or 4 nitrogen atoms. In some embodiments, B is a pyrazolopyrimidine. In some embodiments, in addition to —R²¹, B is optionally substituted with 1, 2, 3, 4, or 5 substituents independently selected from halo, C₁-C₄ alkyl, C₁-C₄ haloalkyl, —OR^{e1}, —N(R^{e2})(R^{e3}), —SO₂R^{e4}, —SO₂N(R^{e5})(R^{e6}), and —NHSO₂R^{e7}, wherein R^{e1}, R^{e2}, R^{e3}, R^{e4}, R^{e5}, R^{e6}, and R^{e7} are each independently selected from hydrogen, C₁-C₄ alkyl, and C₁-C₄ haloalkyl. In some embodiments, in addition to —R²¹, B is substituted with one substituent selected from halo, C₁-C₄ alkyl, C₁-C₄ haloalkyl, —OR^{e1}, —N(R^{e2})(R^{e3}), —SO₂R^{e4}, —SO₂N(R^{e5})(R^{e6}), and —NHSO₂R^{e7}. In some embodiments, in addition to —R²¹, B is substituted with one substituent selected from halo, methyl, trifluoromethyl, —OR^{e1}, —N(R^{e2})(R^{e3}), —SO₂R^{e4}, —SO₂N(R^{e5})(R^{e6}), and —NHSO₂R^{e75}, wherein R^{e1}, R^{e2}, R^{e3}, R^{e4}, R^{e5}, R^{e6}, and R^{e7} are each independently selected from hydrogen, methyl, ethyl, isopropyl, t-butyl, and trifluoromethyl.

In some embodiments, R²¹ is selected from hydrogen and a group of formula:

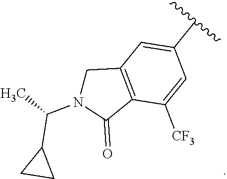 [SMILES: *c1cc2c(c(C(F)(F)F)c1)C(=O)N([C@@H](C)C1CC1)C2]

In some embodiments, L^{b} is —(CR^{c1}R^{c2})ₘ-G^{b}-, wherein m is 0, 1, or 2, R^{c1} and R^{c2} are independently selected from hydrogen and methyl, and G^{b} is a bond, —NHC(O)—, —NH—, —O—, or —S—. In some embodiments, L^{b} has a formula selected from:

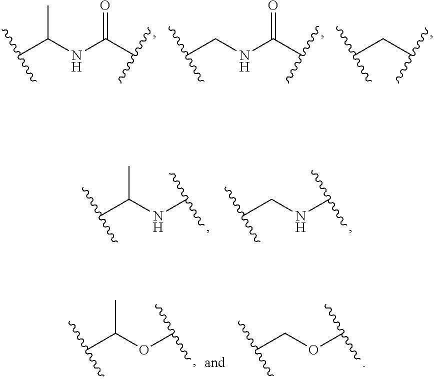

In some embodiments, the moiety of formula (b) is a moiety of formula (bi):

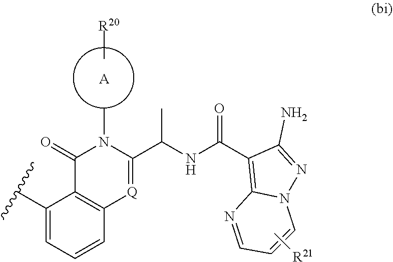

wherein A, Q, R²⁰, and R²¹ are any of the groups defined and described herein.

In some embodiments, the moiety of formula (b) is:

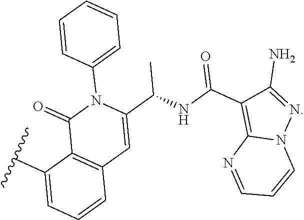 [SMILES: *c1cccc2cc([C@H](C)NC(=O)c3c(N)nn4cccnc34)n(-c3ccccc3)c(=O)c12]

The compound of formula (I) includes L, which is a linker moiety. In some embodiments, L provides sufficient distance between two elements of the compound (i.e., the STING agonist moiety and the group Z, which is either a group of formula (a) (an IDO1 inhibitor) or a group of formula (b) (a PI3K agonist)), to allow each to function undisturbed (or minimally disturbed) by the linkage to the other. In some embodiments, L separates the two groups by about 5 Å to about 1000 Å. In some embodiments, L separates the two groups by 5 Å, 10 Å, 20 Å, 50 Å, 100 Å, 150 Å, 200 Å, 300 Å, 400 Å, 500 Å, 600 Å, 700 Å, 800 Å, 900 Å, 1000 Å, or any suitable range therebetween (e.g., 5-100 Å, 50-500 Å, 150-700 Å, etc.). In some embodiments, L separates two groups by about 1-200 atoms (e.g., 1, 2, 3, 4, 5, 6, 7, 8, 9, 10, 15, 20, 25, 30, 35, 40, 45, 50, 60, 70, 80, 90, 100, 120, 140, 160, 180, 200, or any suitable ranges therebetween (e.g., 2-20, 10-50, etc.)).

L can include one or more groups independently selected from —C(R′)₂—, —CH═CH—, —C≡C—, —O—, —NR′—, —BR′—, —S—, —C(O)—, —C(NR′)—, —S(O)—, —S(O)₂—, arylene, heteroarylene, cycloalkylene, and heterocyclylene, wherein each R′ is independently selected from hydrogen, C₁-C₈₀ alkyl, C₂-C₈₀ alkenyl, C₂-C₈₀ alkynyl, aryl, arylalkyl, cycloalkyl, cycloalkylalkyl, heterocyclyl, heterocyclylalkyl, heteroaryl, heteroarylalkyl, and a lipid moiety, and wherein each alkyl, arylene, heteroarylene, cycloalkylene, and heterocyclylene is independently unsubstituted or substituted with 1, 2, or 3 substituents. In some embodiments, L comprises one or more groups independently selected from —C(R′)₂—, —C≡C—, —O—, —NH—, —C(O)—, and heteroarylene (e.g., a five-membered heteroarylene having 1, 2, or 3 nitrogen atoms), wherein each R′ is independently selected from hydrogen, C₁-C₄₀ alkyl, phenyl, and —CH₂-heterocyclyl (e.g., wherein the heterocyclyl is a 6-membered heterocyclyl having 1 or 2 heteroatoms independently selected from N, O, and S), wherein the phenyl and the heterocyclyl are each independently unsubstituted or substituted with 1 or 2 substituents.

In some embodiments, L comprises one or more —(CH) CH₂O)— (oxyethylene) groups, e.g., 1-20 —(CH₂CH₂O)— groups (e.g., 1, 2, 3, 4, 5, 6, 7, 8, 9, 10, 11, 12, 13, 14, 15, 16, 17, 18, 19, or 20 —(CH₂CH₂O)— groups, or any range therebetween). In some embodiments, L comprises a —(CH₂CH₂O)—, —(CH₂CH₂O)₂—, —(CH₂CH₂O)₃—, —(CH₂CH₂O)₄—, —(CH₂CH₂O)₅—, —(CH₂CH₂O)₆—, —(CH₂CH₂O)₇—, —(CH₂CH₂O)₈—, —(CH₂CH₂O)₉—, or —(CH₂CH₂O)₁₀— group.

In some embodiments, L comprises one or more alkylene groups (e.g., —(CH₂)ₙ, wherein n is 1, 2, 3, 4, 5, 6, 7, 8, 9, 10, 11, or 12). In some embodiments, L comprises one or more branched alkylene groups. In some embodiments, L comprises at least one —C(O)NH— group. In some embodiments, L comprises at least one —O— group. In some embodiments, L comprises at least one five-membered heteroarylene group having 2 or 3 nitrogen atoms (e.g., a group of formula

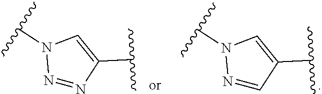 [SMILES: *c1cn(*)nn1 ; *c1cnn(*)c1]

In some embodiments, the groups —X¹-L- together have a formula —O—(CR′₂)ₙ—O—, wherein nis 1, 2, 3, 4, 5, 6, 7, 8, 9, 10, 11, or 12, and each R′ is independently selected from hydrogen, C₁-C₈₀ alkyl, C₂-C₈₀ alkenyl, C₂-C₈₀ alkynyl, aryl, arylalkyl, cycloalkyl, cycloalkylalkyl, heterocyclyl, heterocyclylalkyl, heteroaryl, heteroarylalkyl, and a lipid moiety, and wherein each alkyl, arylene, heteroarylene, cycloalkylene, and heterocyclylene is independently unsubstituted or substituted with 1, 2, or 3 substituents. In some embodiments, L has a formula —O—(CR′₂)ₙ—O—, wherein n is 1, 2, 3, 4, or 5, one R′ is selected from C₁-C₈₀ alkyl, C₂-C₈₀ alkenyl, C₂-C₈₀ alkynyl, aryl, arylalkyl, cycloalkyl, cycloalkylalkyl, heterocyclyl, heterocyclylalkyl, heteroaryl, and heteroarylalkyl, and the remaining R′ are all hydrogen.

In some embodiments, L comprises one or more substituents, pendants, side chains, etc., comprising any suitable organic functional groups (e.g., —OH, —NH₂, —SH, —CN, ═O, ═S, halogen (e.g., —F, —Cl, —Br, —I), —COOH, —CONH₂, —CH₃, etc.).

In some embodiments, the groups —X¹-L- together have a formula selected from:

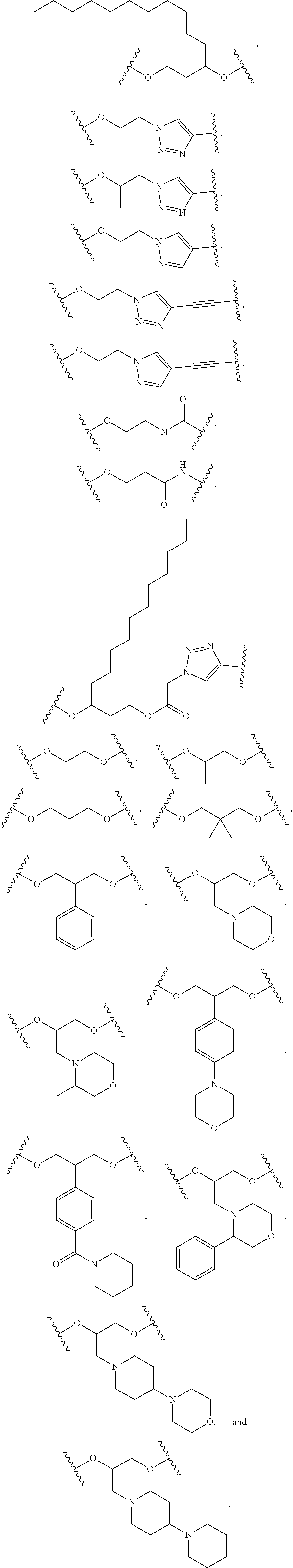

In some embodiments, the compound of formula (I) includes a lipid moiety having at least 8 carbon atoms. For example, in some embodiments, one of R³, R⁴, R⁵, R⁶, and R⁷ is a group —Y—R⁸, wherein R⁸ is a lipid moiety having at least 8 carbon atoms. In some embodiments, the linker group L includes a lipid moiety (e.g., as part of a group —C(R′)₂—, wherein one R′ is a lipid moiety.

The lipid moiety can be derived from any suitable lipid, such as a fatty alcohol, a fatty acid, a phospholipid, a steroid, or a synthetic lipid. In some embodiments, the lipid moiety is derived from a lipid having a functional group, such as a hydroxy group, a carboxylic acid group, or an amino group, and the lipid moiety is attached to the compound of formula (I) via that functional group. For example, in some embodiments, R¹ is a lipid moiety having at least 8 carbon atoms, and in such embodiments, the group X¹ in formula (I) is derived from the functional group; for example, if the lipid moiety is derived from a fatty alcohol, X¹ is O.

In some embodiments, the lipid moiety is selected from C₈-C₈₀ alkyl, C₈-C₈₀ alkenyl, C₈-C₈₀ alkynyl, C₈-C₈₀ heteroalkyl, C₈-C₈₀ heteroalkenyl, and C₈-C₈₀ heteroalkynyl, each of which is optionally substituted with one or more substituents selected from hydroxy and amino.

For example, in some embodiments, the lipid moiety is selected from C₈-C₈₀ alkyl and C₈-C₈₀ alkenyl. In some embodiments, the lipid moiety is selected from C₅-C₄₀ alkyl and C₈-C₄₀ alkenyl. In some embodiments, the lipid moiety is selected from C₁₂-C₄₀ alkyl and C₁₂-C₄₀ alkenyl. In some such embodiments (e.g., wherein R¹ is a lipid moiety and X¹ is O, or wherein R⁸ is a lipid moiety and Y is-C(O)(O—), the lipid moiety is derived from a saturated or unsaturated fatty alcohol. In some embodiments, the lipid moiety is derived from linoleyl alcohol ((9Z,12Z)-octadeca-9,12-dien-1-ol), myristyl alcohol (1-tetradecanol), palmitoleyl alcohol ((Z)-hexadec-9-en-1-ol), oleyl alcohol ((Z)-octadec-9-en-1-ol), elaidyl alcohol (trans-9-octadecenol), cis-vaccenyl alcohol (cis-11-octadecenol), gadoleyl alcohol ((Z)-icos-9-en-1-ol), 11-eicosenol, erucyl alcohol (cis-13-docosenol), 15-tetracosen-1-ol, eicosadienyl alcohol (icosa-11,14-dien-1-ol), linolenyl alcohol ((9Z,12Z,15Z)-9,12,15-octadecatrien-1-ol), Y-linolenyl alcohol ((6E,9E,12E)-octadeca-6,9,12-trien-1-ol), eleostearyl alcohol (octadeca-9,11,13-trien-1-ol), icosa-5,8,11-trien-1-ol, eicos-13-en-1-ol, icosa-11,14-17-trien-1-ol, octadeca-6,9,12,15-tetraen-1-ol, arachidonyl alcohol ((5Z,8Z,11Z,14Z)-icosatetraen-1-ol), 4E,6Z-hexadecadien-1-ol, icosa-5,8,11,14,17-pentaen-1-ol, docosahexaenoyl alcohol (docosa-4,7,10,13,16,19-hexaen-1-ol), docosa-7,10,13,16,19-pentaen-1-ol, tetracosa-6,9,12,15,18,21-hexaen-1-ol, capryl alcohol (1-octanol), pelargonic alcohol (1-nonanol), decyl alcohol (1-decanol), undecyl alcohol (1-undecanol), lauryl alcohol (1-dodecanol), tridecyl alcohol (1-tridecanol), myristyl alcohol (1-tetradecanol), pentadecyl alcohol (1-pentadecanol), cetyl alcohol (1-hexadecanol), palmitoleyl alcohol (cis-9-hexadecen-1-ol), heptadecylalcohol (1-n-heptadecanol), stearyl alcohol (1-octadecanol), oleyl alcohol (1-octadecenol), nonadecyl alcohol (1-nonadecanol), arachidyl alcohol (1-eicosanol), heneicosyl alcohol (1-beneicosanol), behenyl alcohol (1-docosanol), erucyl alcohol (cis-13-docosen-1-ol), 1-tricosanol, lignoceryl alcohol (1-tetracosanol), pentacosylic alcohol (1-pentacosanol), ceryl alcohol (1-hexacosanol), 1-heptacosanol, montanyl alcohol (1-octacosanol), 1-nonacosanol, myricyl alcohol (1-triacontanol), 1-hentriacontanol, 1-dotriacontanol (lacceryl alcohol), 1-tritriacontanol, geddyl alcohol (1-tetratriacontanol), 1-hexatriacontanol, 1-heptatriacontanol, 1-octatriacontanol, nonatriacontan-1-ol, or 1-tetracontanol.

In some embodiments, the lipid moiety is selected from C₈-C₈₀ heteroalkyl, C₈-C₈₀ heteroalkenyl, and C₈-C₈₀ heteroalkynyl. In such embodiments, the lipid moiety can be derived from a lipid including one or more heteroatom groups, such as —O—, —NH—, —C(O)—, or the like, or combinations thereof (e.g., —C(O)O— groups).

For example, in some embodiments, the lipid moiety the lipid moiety has a formula (A):

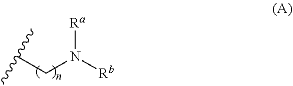

wherein:
n is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40; and
R^{a} and R^{b} are each independently selected from C₆-C₄₀ alkyl, C₆-C₄₀ alkenyl, C₆-C₄₀ heteroalkyl, and C₆-C₄₀ heteroalkenyl.

For example, in some embodiments, n is 1, 2, 3, 4, 5, 6, 7, or 8. In some embodiments, n is 2, 3, or 4. In some embodiments, n is 2. In some embodiments, n is 3. In some embodiments, n is 4.

In some embodiments, R^{a} and R^{b} are each independently selected from C₆-C₄₀ alkyl and C₆-C₄₀ alkenyl. For example, in some embodiments, R^{a} and R^{b} are each independently selected from n-hexyl, n-heptyl, n-octyl, n-nonyl, n-decyl, undecyl, dodecyl, tridecyl, tetradecyl, pentadecyl, hexadecyl, heptadecyl, octadecyl, nonadecyl, icosyl, benicosyl, docosyl, tricosyl, tetracosyl, pentacosyl, hexacosyl, heptacosyl, octacosyl, nonacosyl, triacontyl, hentriacontyl, dotriacontyl, tritriacontyl, tetratriacontyl, pentatriacontyl, hexatriacontyl, heptatriacontyl, octatriacontyl, nonatriacontyl, tetracontyl, linoleyl ((9Z,12Z)-octadeca-9,12-dien-1-yl), palmitoleyl ((Z)-hexadec-9-en-1-yl), oleyl ((Z)-octadec-9-en-1-yl), elaidyl (trans-9-octadecenyl), cis-vaccenyl (cis-11-octadecenyl), gadoleyl ((Z)-icos-9-en-1-yl), 11-eicosenyl, erucyl (cis-13-docosenyl), 15-tetracosen-1-yl, eicosadienyl (icosa-11,14-dien-1-yl), linolenyl ((9Z,12Z,15Z)-9,12,15-octadecatrien-1-yl), γ-linolenyl ((6E,9E,12E)-octadeca-6,9,12-trien-1-yl), eleostearyl (octadeca-9,11,13-trien-1-yl), icosa-5,8,11-trien-1-yl, eicos-13-en-1-yl, icosa-11,14-17-trien-1-yl, octadeca-6,9,12,15-tetraen-1-yl, arachidonyl ((5Z,8Z,11Z,14Z)-icosatetraen-1-yl), 4E,6Z-hexadecadien-1-yl, icosa-5,8,11,14,17-pentaen-1-yl, docosahexaenoyl (docosa-4,7,10,13,16,19-hexaen-1-yl), docosa-7,10,13,16,19-pentaen-1-yl, and tetracosa-6,9,12,15,18,21-hexaen-1-yl. In some embodiments, R^{a} and R^{b} are each linoleyl.

In some embodiments, the lipid moiety has a formula (B) or (C):

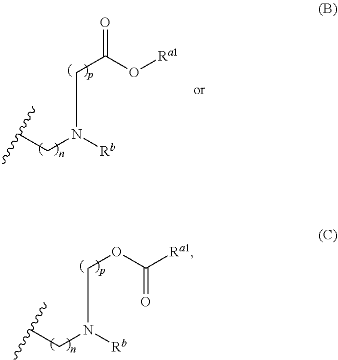

wherein:
n and p are each independently 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40;
R^{a1} is selected from C₆-C₄₀ alkyl and C₆-C₄₀ alkenyl; and
R^{b} is selected from C₆-C₄₀ alkyl, C₆-C₄₀ alkenyl, C₆-C₄₀ heteroalkyl, and C₆-C₄₀ heteroalkenyl.

In some embodiments, n is 1, 2, 3, 4, 5, 6, 7, or 8. In some embodiments, n is 2, 3, or 4. In some embodiments, n is 2. In some embodiments, n is 3. In some embodiments, n is 4.

In some embodiments, p is 1, 2, 3, 4, 5, 6, 7, or 8. In some embodiments, p is 3, 4, 5, 6, or 7. In some embodiments, p is 3. In some embodiments, p is 4. In some embodiments, p is 5. In some embodiments, p is 6. In some embodiments, p is 7.

In some embodiments, R^{a1} is C₆-C₂₄ alkyl or C₆-C₂₄ alkenyl. In some embodiments, R^{a1} is selected from C₉-C₂₂ alkyl and C₉-C₂₂ alkenyl. In some embodiments, R^{a1} is selected from straight or branched C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, or C₂₄ alkyl. In some embodiments, R^{a1} is selected from straight or branched C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, or C₂₄ alkenyl. In some embodiments, R^{a1} is selected from linoleyl, n-nonyl, n-undecyl, henicosan-11-yl, pentadecane-7-yl, and heptadecan-9-yl.

In some embodiments, R^{b} is selected from C₆-C₄₀ alkyl and C₆-C₄₀ alkenyl. For example, in some embodiments, R^{b} is selected from n-hexyl, n-heptyl, n-octyl, n-nonyl, n-decyl, undecyl, dodecyl, tridecyl, tetradecyl, pentadecyl, hexadecyl, heptadecyl, octadecyl, nonadecyl, icosyl, henicosyl, docosyl, tricosyl, tetracosyl, pentacosyl, hexacosyl, heptacosyl, octacosyl, nonacosyl, triacontyl, hentriacontyl, dotriacontyl, tritriacontyl, tetratriacontyl, pentatriacontyl, hexatriacontyl, heptatriacontyl, octatriacontyl, nonatriacontyl, tetracontyl, linoleyl ((9Z,12Z)-octadeca-9,12-dien-1-yl), palmitoleyl ((Z)-hexadec-9-en-1-yl), oleyl ((Z)-octadec-9-en-1-yl), elaidyl (trans-9-octadecenyl), cis-vaccenyl (cis-11-octadecenyl), gadoleyl ((Z)-icos-9-en-1-yl), 11-eicosenyl, erucyl (cis-13-docosenyl), 15-tetracosen-1-yl, eicosadienyl (icosa-11,14-dien-1-yl), linolenyl ((9Z,12Z,15Z)-9,12,15-octadecatrien-1-yl), Y-linolenyl ((6E,9E,12E)-octadeca-6,9,12-trien-1-yl), eleostearyl (octadeca-9,11,13-trien-1-yl), icosa-5,8,11-trien-1-yl, eicos-13-en-1-yl, icosa-11, 14-17-trien-1-yl, octadeca-6,9,12,15-tetraen-1-yl, arachidonyl ((5Z,8Z,11Z,14Z)-icosatetraen-1-yl), 4E,6Z-hexadecadien-1-yl, icosa-5,8,11,14,17-pentaen-1-yl, docosahexaenoyl (docosa-4,7,10,13,16,19-hexaen-1-yl), docosa-7,10,13,16,19-pentaen-1-yl, and tetracosa-6,9,12,15,18,21-hexaen-1-yl. In some embodiments, R^{b} is linoleyl.

In some embodiments, the lipid moiety has a formula (D), (E), or (F):

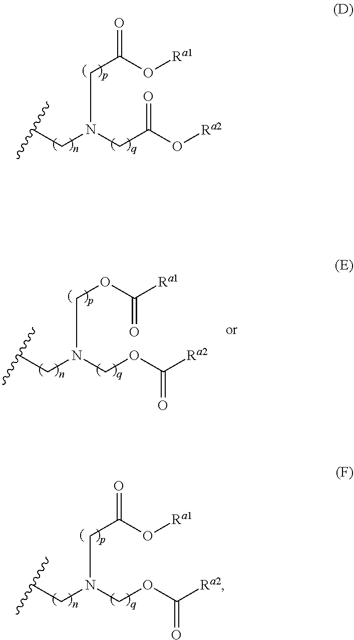

wherein:
n, p, and q are each independently 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40; and
R^{a1} and R^{a2} are each independently selected from C₆-C₄₀ alkyl and C₆-C₄₀ alkenyl.

In some embodiments, the lipid moiety has formula (D). In some embodiments, the lipid moiety has formula (E). In some embodiments, the lipid moiety has formula (F). In some embodiments, n is 1, 2, 3, 4, 5, 6, 7, or 8. In some embodiments, n is 2, 3, or 4. In some embodiments, n is 2. In some embodiments, n is 3. In some embodiments, n is 4.

In some embodiments, p and q are each independently selected from 1, 2, 3, 4, 5, 6, 7, or 8. In some embodiments, p and q are each independently selected from 3, 4, 5, 6, and 7. In some embodiments, p and q are each independently selected from 3, 5, and 7. In some embodiments, p and q are each 5. In some embodiments, p and q are each 6. In some embodiments, p and q are each 7.

In some embodiments, R^{a1} and R^{a2} are each independently selected from C₆-C₂₄ alkyl and C₆-C₂₄ alkenyl. In some embodiments, R^{a1} and R^{a2} are each independently selected from C₉-C₂₂ alkyl and C₉-C₂₂ alkenyl. In some embodiments, R^{a1} and R^{a2} are each independently selected from straight or branched C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, or C₂₄ alkyl. In some embodiments, R^{a1} and R^{a2} are each independently selected from straight or branched C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, or C₂₄ alkenyl. In some embodiments, R^{a1} and R^{a2} are each independently selected from linoleyl, n-nonyl, n-undecyl, henicosan-11-yl, pentadecane-7-yl, and heptadecan-9-yl.

In some embodiments, the lipid moiety is derived from a steroid. For example, in some embodiments, the lipid moiety is derived from cholesterol, beta-sistesterol, or BHEM-cholesterol.

In some embodiments, the lipid moiety is selected from:

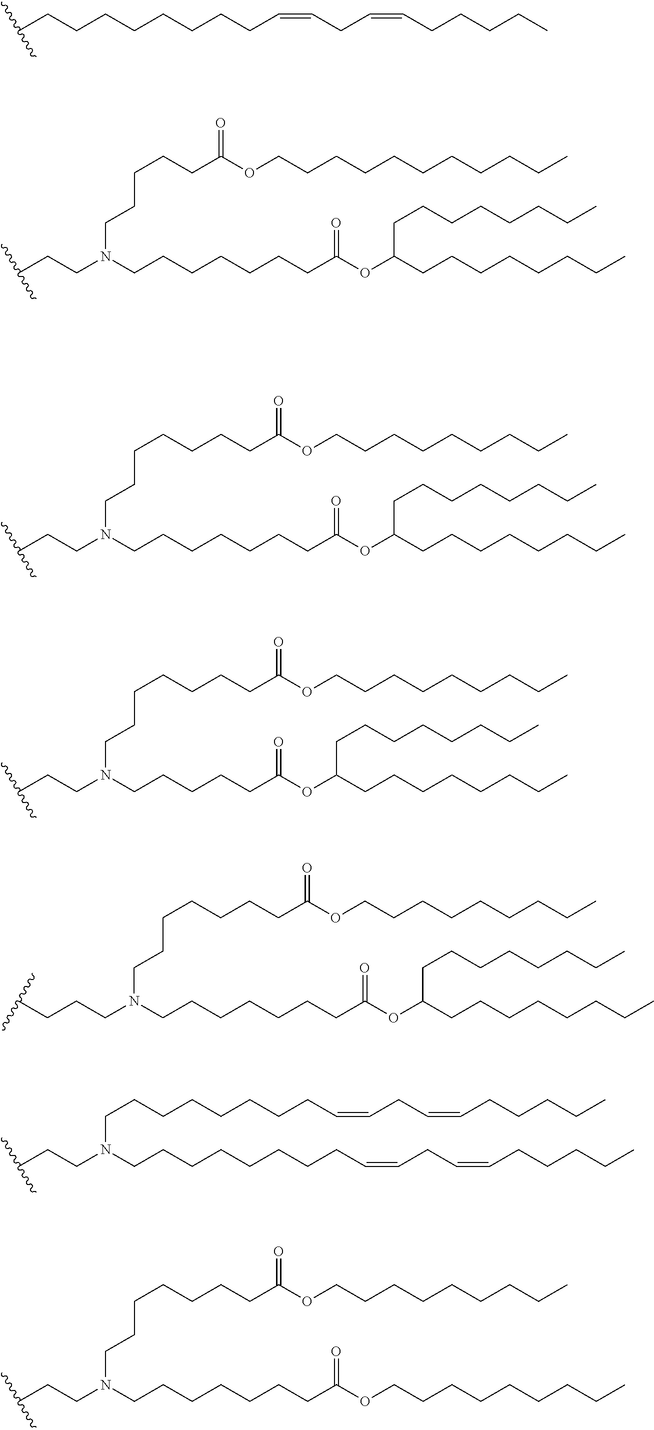

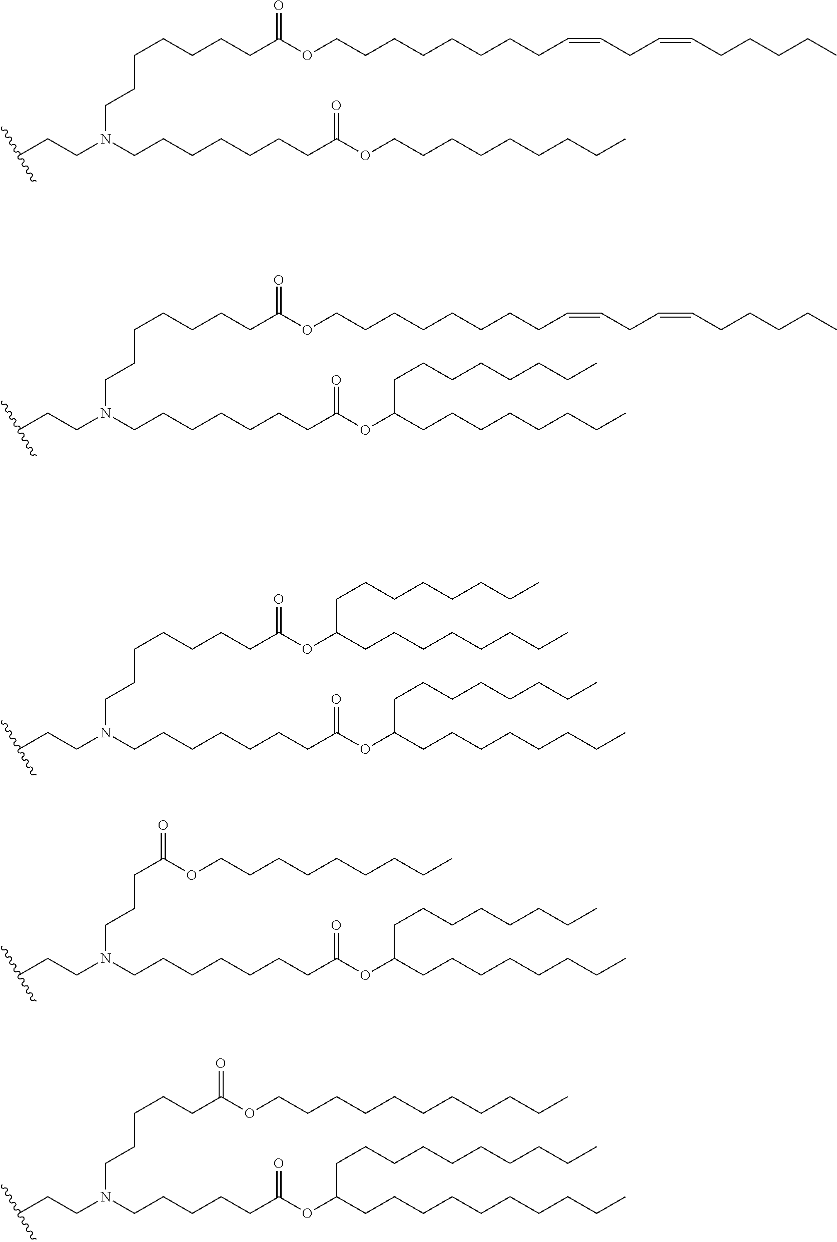 [SMILES: *CCN(CCCCCCCC(=O)OCCCCCCCCC)CCCCCCCC(=O)OCCCCCCCC/C=C\C/C=C\CCCCC ; *CCN(CCCCCCCC(=O)OCCCCCCCC/C=C\C/C=C\CCCCC)CCCCCCCC(=O)OC(CCCCCCCC)CCCCCCCC ; *CCN(CCCCCCCC(=O)OC(CCCCCCCC)CCCCCCCC)CCCCCCCC(=O)OC(CCCCCCCC)CCCCCCCC ; *CCN(CCCCCCCC(=O)OC(CCCCCCCC)CCCCCCCC)CCCC(=O)OCCCCCCCCC ; *CCN(CCCCCC(=O)OCCCCCCCCCCC)CCCCCC(=O)OC(CCCCCCCCCC)CCCCCCCCCC]

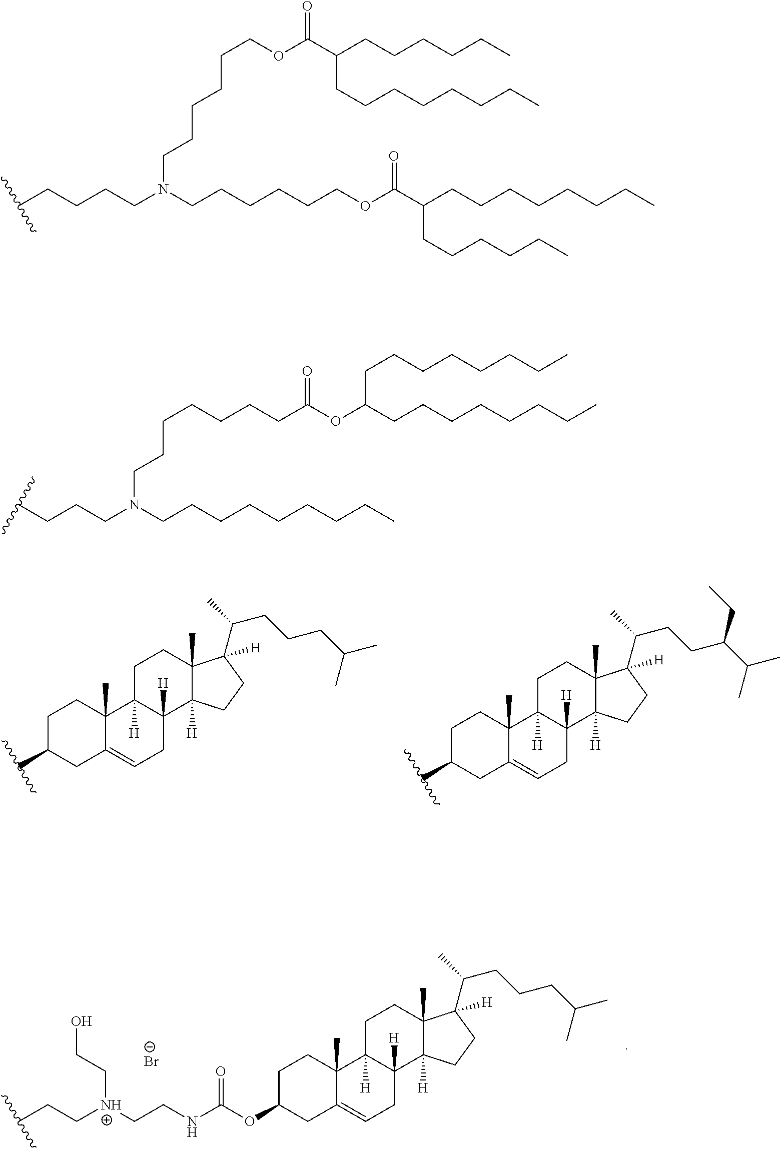

In some embodiments, the compound of formula (I) is selected from:

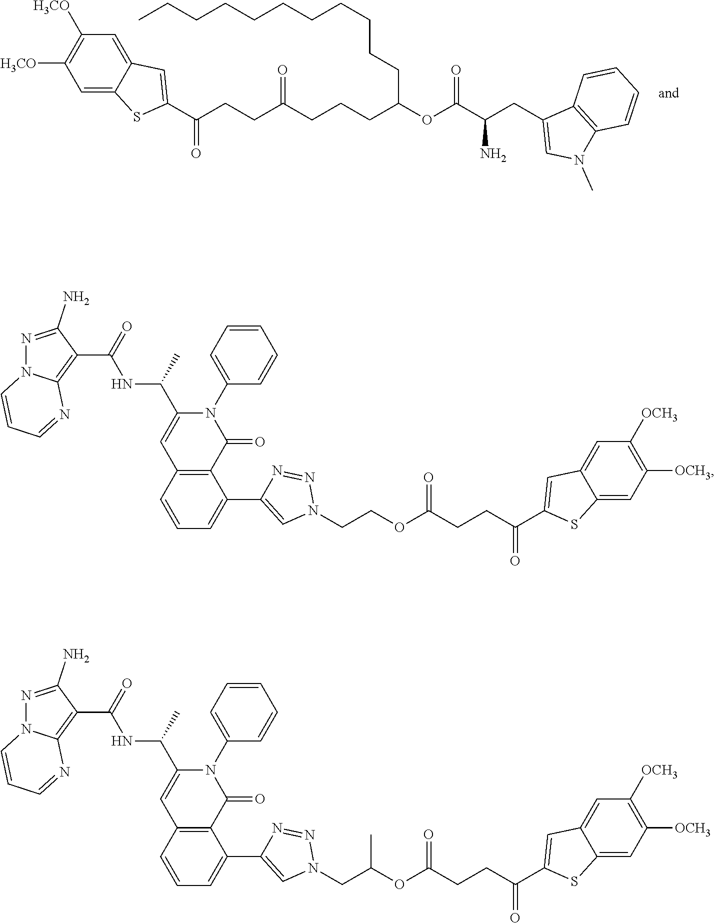 [SMILES: CCCCCCCCCCCC(CCCC(=O)CCC(=O)c1cc2cc(OC)c(OC)cc2s1)OC(=O)[C@H](N)Cc1cn(C)c2ccccc12 ; COc1cc2cc(C(=O)CCC(=O)OCCn3cc(-c4cccc5cc([C@@H](C)NC(=O)c6c(N)nn7cccnc67)n(-c6ccccc6)c(=O)c45)nn3)sc2cc1OC ; COc1cc2cc(C(=O)CCC(=O)OC(C)Cn3cc(-c4cccc5cc([C@@H](C)NC(=O)c6c(N)nn7cccnc67)n(-c6ccccc6)c(=O)c45)nn3)sc2cc1OC]

and pharmaceutically acceptable salts thereof.

Other compounds of formula (I) include:

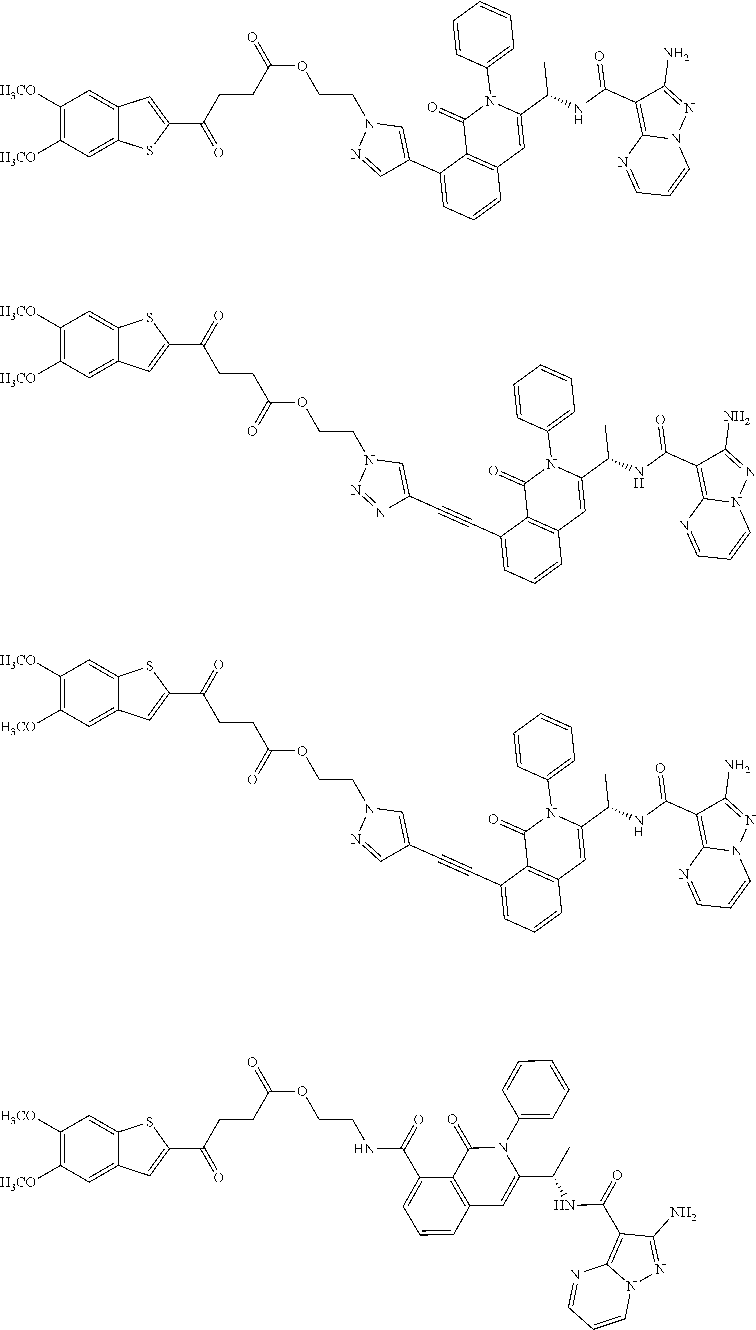 [SMILES: COc1cc2cc(C(=O)CCC(=O)OCCn3cc(-c4cccc5cc([C@H](C)NC(=O)c6c(N)nn7cccnc67)n(-c6ccccc6)c(=O)c45)cn3)sc2cc1OC ; COc1cc2cc(C(=O)CCC(=O)OCCn3cc(C#Cc4cccc5cc([C@H](C)NC(=O)c6c(N)nn7cccnc67)n(-c6ccccc6)c(=O)c45)nn3)sc2cc1OC ; COc1cc2cc(C(=O)CCC(=O)OCCn3cc(C#Cc4cccc5cc([C@H](C)NC(=O)c6c(N)nn7cccnc67)n(-c6ccccc6)c(=O)c45)cn3)sc2cc1OC ; COc1cc2cc(C(=O)CCC(=O)OCCNC(=O)c3cccc4cc([C@H](C)NC(=O)c5c(N)nn6cccnc56)n(-c5ccccc5)c(=O)c34)sc2cc1OC]

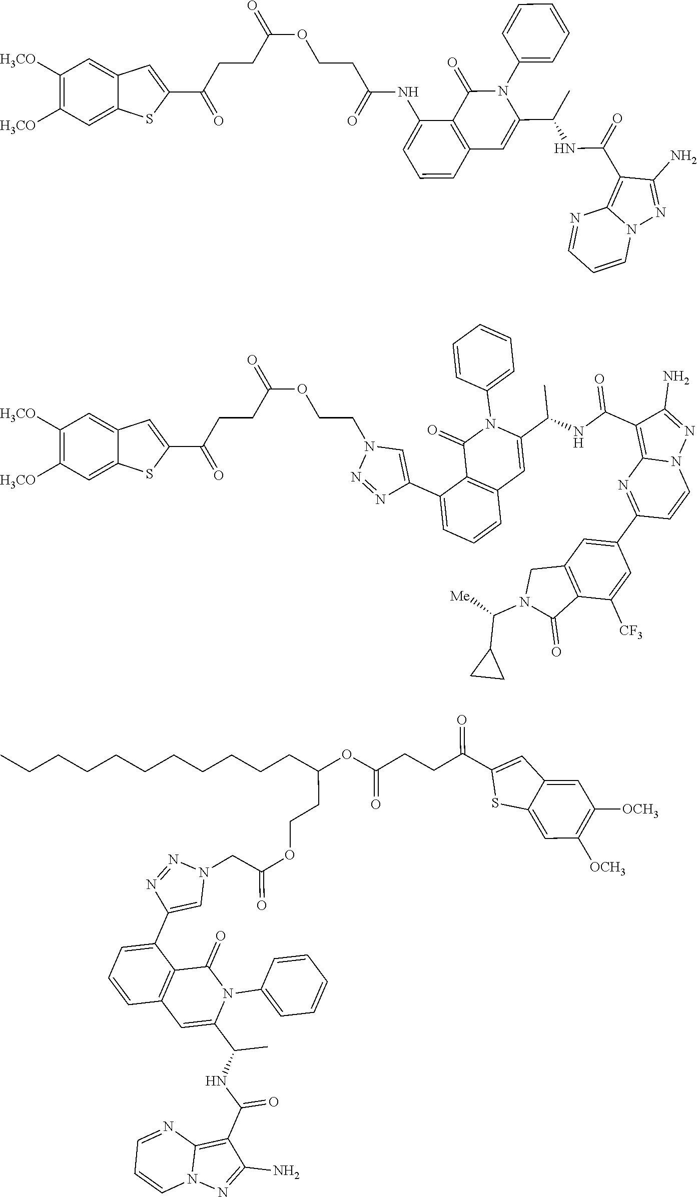

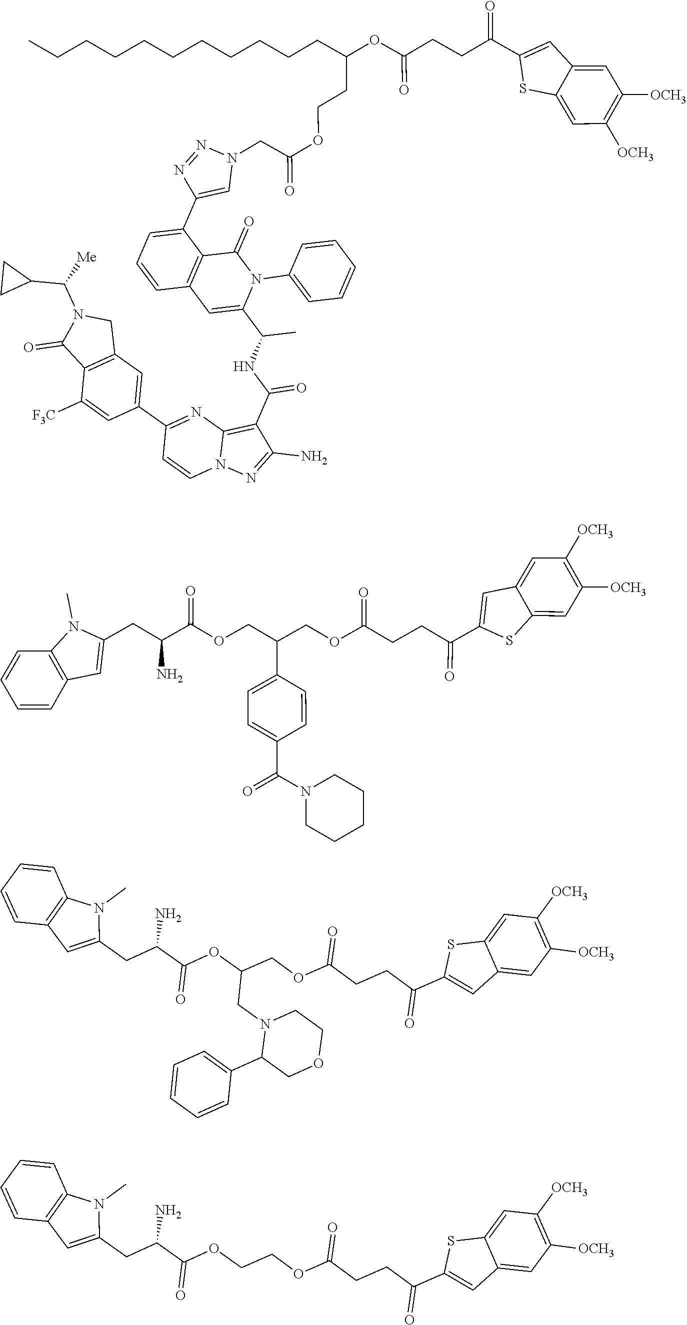 [SMILES: CCCCCCCCCCCC(CCOC(=O)Cn1cc(-c2cccc3cc([C@H](C)NC(=O)c4c(N)nn5ccc(-c6cc7c(c(C(F)(F)F)c6)C(=O)N([C@@H](C)C6CC6)C7)nc45)n(-c4ccccc4)c(=O)c23)nn1)OC(=O)CCC(=O)c1cc2cc(OC)c(OC)cc2s1 ; COc1cc2cc(C(=O)CCC(=O)OCC(COC(=O)[C@@H](N)Cc3cc4ccccc4n3C)c3ccc(C(=O)N4CCCCC4)cc3)sc2cc1OC ; COc1cc2cc(C(=O)CCC(=O)OCC(CN3CCOCC3c3ccccc3)OC(=O)[C@@H](N)Cc3cc4ccccc4n3C)sc2cc1OC ; COc1cc2cc(C(=O)CCC(=O)OCCOC(=O)[C@@H](N)Cc3cc4ccccc4n3C)sc2cc1OC]

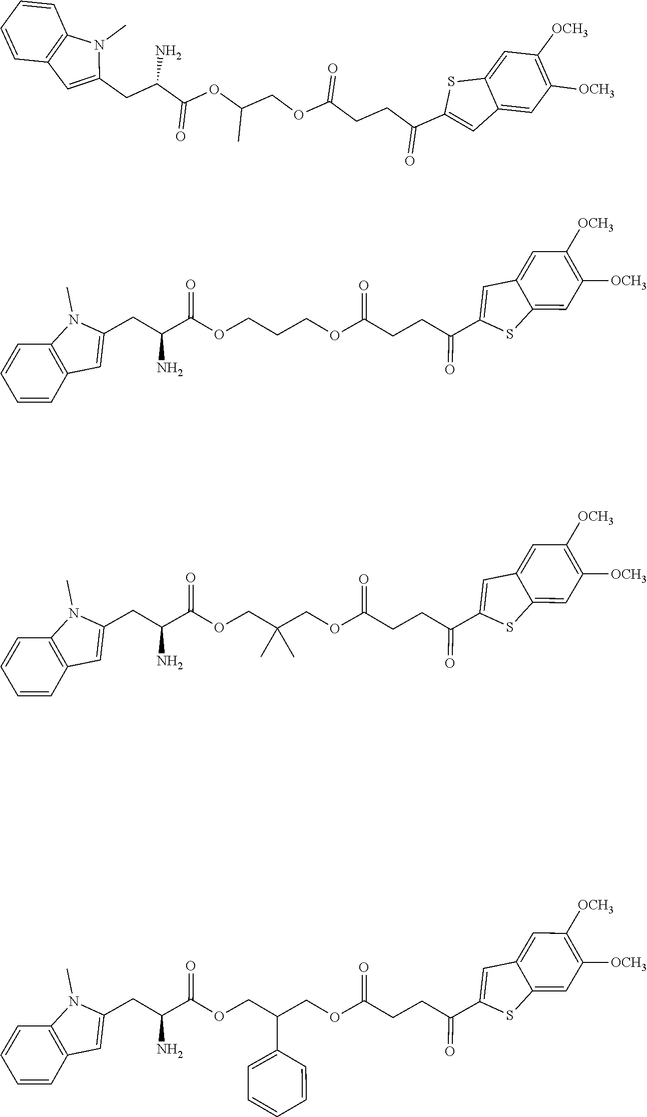 [SMILES: COc1cc2cc(C(=O)CCC(=O)OCC(C)OC(=O)[C@@H](N)Cc3cc4ccccc4n3C)sc2cc1OC ; COc1cc2cc(C(=O)CCC(=O)OCCCOC(=O)[C@@H](N)Cc3cc4ccccc4n3C)sc2cc1OC ; COc1cc2cc(C(=O)CCC(=O)OCC(C)(C)COC(=O)[C@@H](N)Cc3cc4ccccc4n3C)sc2cc1OC ; COc1cc2cc(C(=O)CCC(=O)OCC(COC(=O)[C@@H](N)Cc3cc4ccccc4n3C)c3ccccc3)sc2cc1OC]

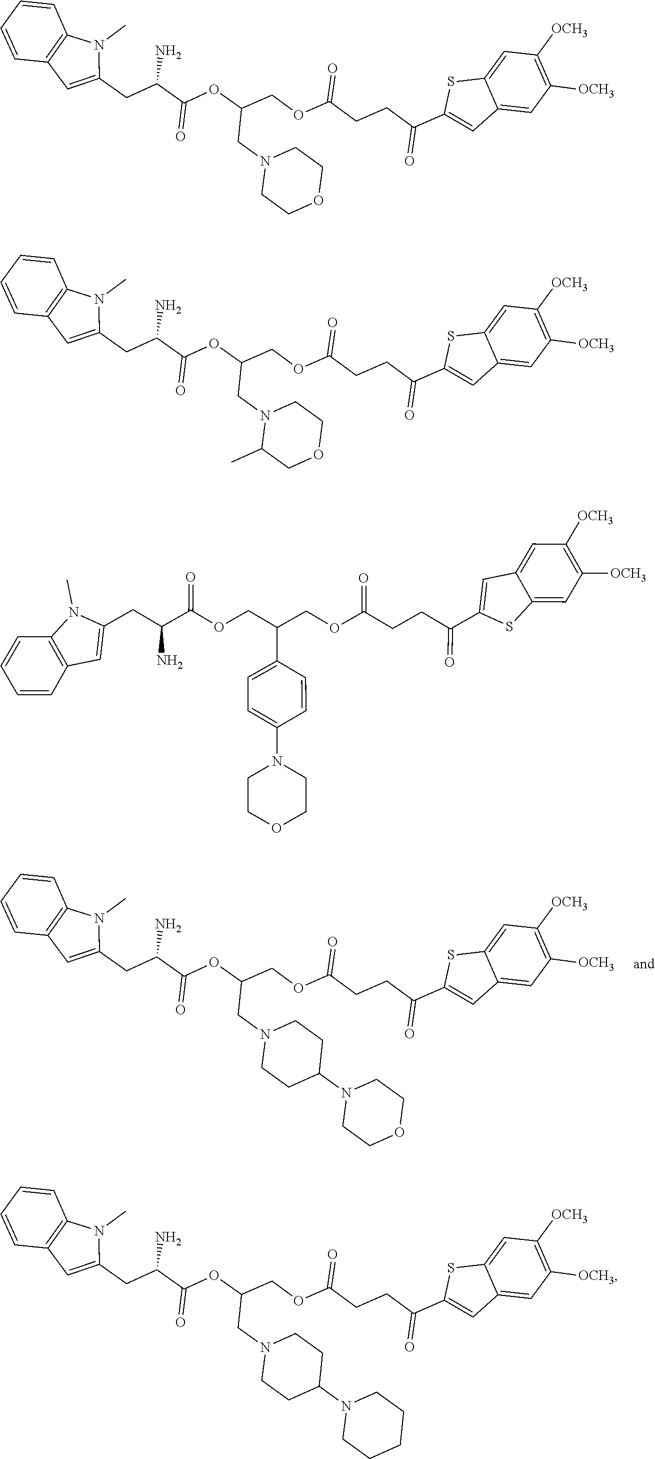 [SMILES: COc1cc2cc(C(=O)CCC(=O)OCC(CN3CCOCC3)OC(=O)[C@@H](N)Cc3cc4ccccc4n3C)sc2cc1OC ; COc1cc2cc(C(=O)CCC(=O)OCC(CN3CCOCC3C)OC(=O)[C@@H](N)Cc3cc4ccccc4n3C)sc2cc1OC ; COc1cc2cc(C(=O)CCC(=O)OCC(COC(=O)[C@@H](N)Cc3cc4ccccc4n3C)c3ccc(N4CCOCC4)cc3)sc2cc1OC ; COc1cc2cc(C(=O)CCC(=O)OCC(CN3CCC(N4CCOCC4)CC3)OC(=O)[C@@H](N)Cc3cc4ccccc4n3C)sc2cc1OC ; COc1cc2cc(C(=O)CCC(=O)OCC(CN3CCC(N4CCCCC4)CC3)OC(=O)[C@@H](N)Cc3cc4ccccc4n3C)sc2cc1OC]

or a pharmaceutically acceptable salt thereof.

Also disclosed herein are compounds of formula (II):

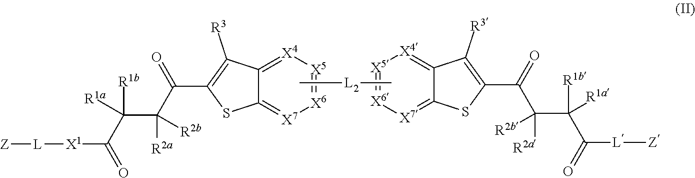

or a pharmaceutically acceptable salt thereof, wherein:
R^{1a}, R^{1b}, R^{2a}, R^{2b}, R^{1a′}, R^{1b′}, R^{2a′}, and R^{2b′} are each independently selected from hydrogen, C₁-C₄ alkyl, C₁-C₄ alkoxy, C₃-C₆ cycloalkyl, halo-C₁-C₄-alkyl, amino-C₁-C₄-alkyl, hydroxy-C₁-C₄-alkyl, C₁-C₄-alkoxy-C₁-C₄-alkyl, halo, hydroxy, amino, C₁-C₄-alkylamino, di-C₁-C₄-alkylamino, and cyano; wherein R^{1a} and R^{1b}, or R^{1a} and R^{2a}, or R²ª and R^{2b}, are optionally taken together with the carbon atom(s) to which they are attached to form an optionally substituted 3- to 6-membered ring; and wherein R^{1a′} and R^{1b′}, or R^{1a′} and R^{2a′}, or R^{2a′} and R^{26′}, are optionally taken together with the carbon atom(s) to which they are attached to form an optionally substituted 3- to 6-membered ring
X⁴ is CR⁴ or N;
X⁵ is CR⁵ or N;
X⁶ is CR⁶ or N;
X⁷ is CR⁷ or N;
X^{4′} is CR^{4′} or N;
X^{5′} is CR^{5′} or N;
X^{6′} is CR^{6′} or N;
X^{7′} is CR^{7′} or N;
R³, R⁴, R⁵, R⁶, R⁷, R^{3′}, R^{4′}, R^{5′}, R^{6′}, and R^{7′} are each independently selected from hydrogen, C₁-C₆ alkyl, C₂-C₆ alkenyl, C₂-C₆ alkynyl, C₁-C₆ alkoxy, C₁-C₆ alkylthio, C₃-C₆ cycloalkyl, halo-C₁-C₆-alkyl, halo-C₁-C₆-alkoxy, amino-C₁-C₆-alkyl, hydroxy-C₁-C₆-alkyl, C₁-C₆-alkoxy-C₁-C₆-alkyl, halo, hydroxy, amino, C₁-C₄-alkylamino, di-C₁-C₄-alkylamino, cyano, —COOR^{x}, —CON(R^{y})₂, —SO₂R^{z}, an oligo- or poly-ethylene glycol chain, and a group —Y—R⁸; wherein R⁴ and R⁵, R⁵ and R⁶, or R⁶ and R⁷ are optionally taken together with the carbon atoms to which they are attached to form an optionally substituted 5- or 6-membered ring; and wherein R^{4′} and R^{5′}, R^{5′} and R^{6′}, or R^{6′} and R^{7′} are optionally taken together with the carbon atoms to which they are attached to form an optionally substituted 5- or 6-membered ring;
Y is selected from —C(O)—, —C(O)O—, —C(O)NR^{v}—, and —C(O)S—;
R⁸ is a lipid moiety having at least 8 carbon atoms;
R^{y}, R^{w}, R^{x}, R^{y}, and R^{z} are each independently selected from hydrogen, C₁-C₆ alkyl, C₂-C₆ alkenyl, C₂-C₆ alkynyl, C₃-C₆ cycloalkyl, and halo-C₁-C₆-alkyl;
L, L′, and L² are each independently a linker; and
Z and Z′ are each independently a moiety of formula (a) or formula (b):

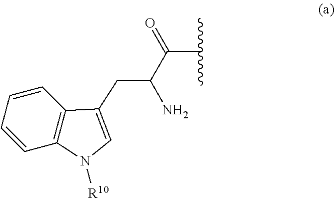

wherein R¹⁰ is hydrogen, or C₁-C₆ alkyl;

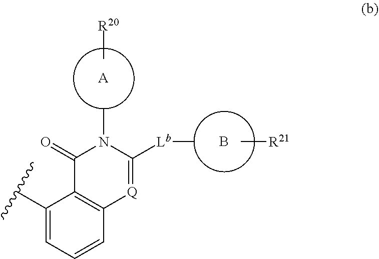

wherein:
Q is CH or N;
A is aryl or a 5- or 6-membered monocyclic heteroaryl having 1, 2, 3, or 4 heteroatoms independently selected from N, O, S, and P;
R²⁰ is selected from hydrogen, halo, C₁-C₄ alkyl, C₃-C₆ cycloalkyl, C₁-C₄ haloalkyl, —OR^{a1}, —N(R^{a2})(R^{a3}), —SO₂R^{a4}, —SO₂N(R^{a5})(R^{a6}), and —NHSO₂R^{a7}, wherein R^{a1}, R^{a2}, R^{a3}, R^{a4}, R^{a5}, R^{a6}, and R^{a7} are each independently selected from hydrogen, C₁-C₄ alkyl, and C₁-C₄ haloalkyl;
R²¹ is selected from hydrogen and a group-L²¹-E, wherein:
   L²¹ is a bond, C₁-C₂ alkylene, —CH═CH—, —C≡C—, —C(O)—, —O—, —NH—, —S—, —C(O)O—, —C(O)NH—, —C(O)S—, arylene, cycloalkylene, heteroarylene, or heterocyclylene, or wherein L²¹ comprises a combination of any two of such groups;
   E is a bicyclic heterocyclyl or bicyclic heteroaryl, each of which is optionally substituted with 1, 2, 3, 4, or 5 substituents independently selected from halo, C₁-C₄ alkyl, C₃-C₆ cycloalkyl, C₃-C₆-cycloalkyl-C₁₋₄-alkyl, C₁-C₄ haloalkyl, oxo, —OR^{b1}, —N(R^{b2})(R^{b3}), —SOR^{b4}, —SO₂N(R^{b5})(R^{b6}), and —NHSO₂R^{b7}, wherein R^{b1}, R^{b2}, R^{b3}, R^{b4}, R^{b5}, R^{b6}, and R^{b7} are each independently selected from hydrogen, C₁-C₄ alkyl, and C₁-C₄ haloalkyl;
L^{b} is —(CR^{c1}R^{c2})ₘ-G^{b}-, wherein:
   R^{c1} and R^{c2} are independently selected from hydrogen and C₁-C₄ alkyl;
   m is 0, 1, or 2; and
   G^{b} is a bond, —NHC(O)—, —NH—, —O—, or —S—; and
B is a bicyclic heteroaryl or bicyclic heterocyclyl, each of which is optionally substituted with 1, 2, 3, 4, or 5 substituents independently selected from halo, C₁-C₄ alkyl, C₃-C₆ cycloalkyl, C₁-C₄ haloalkyl, optionally substituted aryl, —OR^{d1}, —N(R^{d2})(R^{d3}), —SO₂R^{d4}, —SO₂N(R^{d5})(R^{d6}), and —NHSO₂R^{d7}, wherein R^{d1}, R^{d2}, R^{d3}, R^{d4}, R^{d5}, R^{d6}, and R^{d7} are each independently selected from hydrogen, C₁-C₄ alkyl, and C₁-C₄ haloalkyl.

Also included in the scope of this disclosure are compounds that include other STING agonists in place of the group

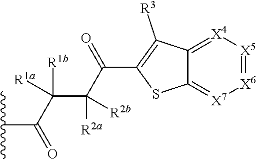

in formula (I). Other STING agonists include, for example, cyclic dinucleotides (CDNs)(Krasteva et al. Nat. Chem. Biol. 2017, 13 (4), 350-359; Burdette et al. Nature 2011, 478 (7370), 515-518), amidobenzimidazole (ABZI) and its derivatives (including diABZI1, diABZI2, and diABZI3) (Ramanjulu et al. Nature 2018, 564, 439-443; Song et al. J. Med. Chem. 2021, 64 (3), 1649-1669), 5,6-dimethylxanthenone-4-acetic acid (DMXAA), 10-carboxymethyl-9-acridanone (CMA), SR-717 (Chin et al. Science 2020, 369 (6506), 993-999), α-mangostin (Zhang et al. ChemMedChem 2018, 13 (19), 2057-2064), Compound 12b (2-methoxy-5,6-dimethylacridin-9(10H)-one; Hou et al. Bioorg. Chem. 2020, 95:103556), Compound G10 (4-(2-chloro-6-fluorobenzyl)-N-(furan-2-ylmethyl)-3-oxo-3,4-dihydro-2H-benzo[b][1,4]thiazine-6-carboxamide; Sali et al. PLoS Pathog. 2015, 11 (12), p. e1005324)), Compound C11 (N-(methylcarbamoyl)-2-phenyl-2-((5-(p-tolyl)-1,3,4-oxadiazol-2-yl) thio) acetamide; Gall et al. J. Virol. 2018, 92 (6)), (5a′S, 10a′R)-5a′,6′-dihydro-3′H,5′H-dispiro[indene-2,2′-dithiazolo[3,2-a:3′,4′-d]pyrazine-8′,2″-indene]-1,1″,3,3″,5′, 10′(10a′H)-hexaone (DSDP; Liu et al. Antiviral Res. 2017, 147, 37-46), and other compounds including ADU-S100, MK-154, MK-2118, BMS-986301, GSK-3745417, SB-11285, and IMSA-101. STING agonists are also disclosed in, e.g., Zhang et al., J. Med. Chem. 2020, 63 (8), 3785-3816.

Certain compounds disclosed herein (e.g., in the examples) may have additional single-function or dual-function activities, e.g., activity as PI3K inhibitors or as dual-function PI3K-IDO compounds. For example, certain compounds prepared as intermediates in the examples herein have individual activity as PI3K inhibitors, including but not limited to the following compounds:

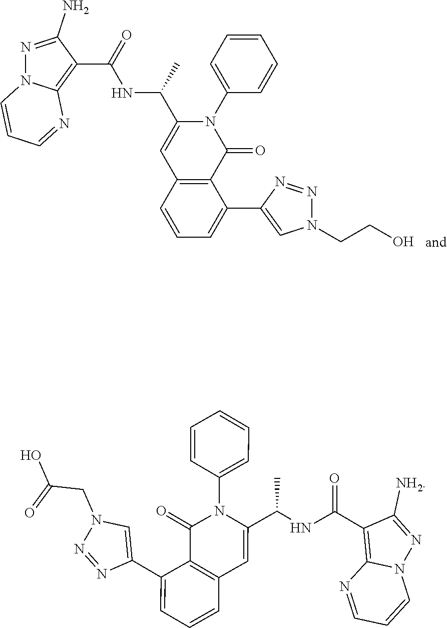 [SMILES: C[C@@H](NC(=O)c1c(N)nn2cccnc12)c1cc2cccc(-c3cn(CCO)nn3)c2c(=O)n1-c1ccccc1 ; C[C@H](NC(=O)c1c(N)nn2cccnc12)c1cc2cccc(-c3cn(CC(=O)O)nn3)c2c(=O)n1-c1ccccc1]

The present disclosure is also intended to encompass such compounds, along with pharmaceutically acceptable salts thereof, pharmaceutical compositions thereof, and methods of use thereof.

The compounds may exist as a stereoisomer wherein asymmetric or chiral centers are present. The stereoisomer is “R” or “S” depending on the configuration of substituents around the chiral carbon atom. The terms “R” and “S” used herein are configurations as defined in IUPAC 1974 Recommendations for Section E, Fundamental Stereochemistry, in Pure Appl. Chem., 1976, 45:13-30. The disclosure contemplates various stereoisomers and mixtures thereof and these are specifically included within the scope of this disclosure. Stereoisomers include enantiomers and diastereomers, and mixtures of enantiomers or diastereomers. Individual stereoisomers of the compounds may be prepared synthetically from commercially available starting materials, which contain asymmetric or chiral centers or by preparation of racemic mixtures followed by methods of resolution well-known to those of ordinary skill in the art. These methods of resolution are exemplified by (1) attachment of a mixture of enantiomers to a chiral auxiliary, separation of the resulting mixture of diastereomers by recrystallization or chromatography and optional liberation of the optically pure product from the auxiliary as described in Furniss, Hannaford, Smith, and Tatchell, “Vogel's Textbook of Practical Organic Chemistry,” 5th edition (1989), Longman Scientific & Technical, Essex CM20 2JE, England (or more recent versions thereof), or (2) direct separation of the mixture of optical enantiomers on chiral chromatographic columns, or (3) fractional recrystallization methods.

It should be understood that the compounds may possess tautomeric forms, as well as geometric isomers, and that these also constitute embodiments of the disclosure.

The present disclosure also includes isotopically-labeled compounds, which is identical to those recited in formula (I), but for the fact that one or more atoms are replaced by an atom having an atomic mass or mass number different from the atomic mass or mass number usually found in nature. Examples of isotopes include those for hydrogen, carbon, nitrogen, oxygen, phosphorus, sulfur, fluorine, and chlorine, such as, but not limited to ²H, ³H, ¹³C, ¹⁴C, ¹⁵N, ¹⁸O, ¹⁷O, ³¹P, ³²P, ³⁵S, ¹⁸F, and ³⁶Cl, respectively. Substitution with heavier isotopes such as deuterium, for example, ²H, can afford certain therapeutic advantages resulting from greater metabolic stability, for example increased in vivo half-life or reduced dosage requirements and, hence, may be preferred in some circumstances. The compound may incorporate positron-emitting isotopes for medical imaging and positron-emitting tomography (PET) studies. Suitable positron-emitting isotopes that can be incorporated in compounds of formula (I) are ¹¹C, ¹³N, ¹⁵O, and ¹⁸F. Isotopically-labeled compounds of formula (I) can generally be prepared by conventional techniques known to those skilled in the art or by processes analogous to those described in the accompanying Examples using appropriate isotopically-labeled reagent in place of non-isotopically-labeled reagent.

The disclosed compounds may exist as pharmaceutically acceptable salts. The term “pharmaceutically acceptable salt” refers to salts or zwitterions of the compounds which are water or oil-soluble or dispersible, suitable for treatment of disorders without undue toxicity, irritation, and allergic response, commensurate with a reasonable benefit/risk ratio and effective for their intended use. The salts may be prepared during the final isolation and purification of the compounds or separately by reacting an amino group of the compounds with a suitable acid. For example, a compound may be dissolved in a suitable solvent, such as but not limited to methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloric, hydrobromic, sulfuric, phosphoric and the like. The amino groups of the compounds may also be quaternized with alkyl chlorides, bromides and iodides such as methyl, ethyl, propyl, isopropyl, butyl, lauryl, myristyl, stearyl and the like.

Basic addition salts may be prepared during the final isolation and purification of the disclosed compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like.

Compounds may be synthesized according to a variety of methods, including those illustrated in the Examples. Reaction conditions and reaction times for each individual step can vary depending on the particular reactants employed and substituents present in the reactants used. Specific procedures are provided in the Examples section. Reactions can be worked up in the conventional manner, e.g., by eliminating the solvent from the residue and further purified according to methodologies generally known in the art such as, but not limited to, crystallization, distillation, extraction, trituration, and chromatography. Unless otherwise described, the starting materials and reagents are either commercially available or can be prepared by one skilled in the art from commercially available materials using methods described in the chemical literature. Starting materials, if not commercially available, can be prepared by procedures selected from standard organic chemical techniques, techniques that are analogous to the synthesis of known, structurally similar compounds, or techniques that are analogous to the above described schemes or the procedures described in the synthetic examples section.

Routine experimentations, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the disclosure. Suitable protecting groups and the methods for protecting and deprotecting different substituents using such suitable protecting groups are well known to those skilled in the art; examples of which can be found in PGM Wuts and TW Greene, in Greene's book titled Protective Groups in Organic Synthesis (4th ed.), John Wiley & Sons, NY (2006), which is incorporated herein by reference in its entirety. Synthesis of the compounds of the disclosure can be accomplished by methods analogous to those described in the synthetic schemes described hereinabove and in specific examples.

When an optically active form of a disclosed compound is required, it can be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization, or enzymatic resolution).

Similarly, when a pure geometric isomer of a compound is required, it can be obtained by carrying out one of the above procedures using a pure geometric isomer as a starting material, or by resolution of a mixture of the geometric isomers of the compound or intermediates using a standard procedure such as chromatographic separation.

It can be appreciated that the synthetic schemes and specific examples as described are illustrative and are not to be read as limiting the scope of the disclosure as it is defined in the appended claims. All alternatives, modifications, and equivalents of the synthetic methods and specific examples are included within the scope of the claims.

### 3. COMPOSITIONS

The disclosed compounds may be incorporated into compositions that may be suitable for administration to a subject (such as a patient, which may be a human or non-human).

a. Pharmaceutical Compositions

The disclosed compounds may be incorporated into pharmaceutically acceptable compositions. The pharmaceutical compositions may include a “therapeutically effective amount” or a “prophylactically effective amount” of the compound(s). A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of a compound of the invention (e.g., a compound of formula (I)) are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The pharmaceutical compositions and formulations may include pharmaceutically acceptable carriers. The term “pharmaceutically acceptable carrier,” as used herein, means a non-toxic, inert solid, semi-solid or liquid filler, diluent, encapsulating material, surfactant, cyclodextrins or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; surfactants such as, but not limited to, cremophor EL, cremophor RH 60, Solutol HS 15 and polysorbate 80; cyclodextrins such as, but not limited to, alpha-CD, beta-CD, gamma-CD, HP-beta-CD, SBE-beta-CD; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The route by which the disclosed compounds are administered and the form of the composition will dictate the type of carrier to be used. The composition may be in a variety of forms, suitable, for example, for systemic administration (e.g., oral, rectal, nasal, sublingual, buccal, implants, or parenteral injections) or topical administration (e.g., dermal, pulmonary, nasal, aural, ocular, liposome delivery systems, or iontophoresis). In some embodiments, the composition is for oral administration.

Carriers for systemic administration typically include at least one of diluents, lubricants, binders, disintegrants, colorants, flavors, sweeteners, antioxidants, preservatives, glidants, solvents, suspending agents, wetting agents, surfactants, cyclodextrins combinations thereof, and others. All carriers are optional in the compositions.

Suitable diluents include sugars such as glucose, lactose, dextrose, and sucrose; diols such as propylene glycol; calcium carbonate; sodium carbonate; sugar alcohols, such as glycerin; mannitol; and sorbitol. The amount of diluent(s) in a systemic or topical composition is typically about 50 to about 90%.

Suitable lubricants include silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of theobroma. The amount of lubricant(s) in a systemic or topical composition is typically about 5 to about 10%.

Suitable binders include polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of binder(s) in a systemic composition is typically about 5 to about 50%.

Suitable disintegrants include agar, alginic acid and the sodium salt thereof, effervescent mixtures, croscarmellose, crospovidone, sodium carboxymethyl starch, sodium starch glycolate, clays, and ion exchange resins. The amount of disintegrant(s) in a systemic or topical composition is typically about 0.1 to about 10%.

Suitable colorants include a colorant such as an FD&C dye. When used, the amount of colorant in a systemic or topical composition is typically about 0.005 to about 0.1%.

Suitable flavors include menthol, peppermint, and fruit flavors. The amount of flavor(s), when used, in a systemic or topical composition is typically about 0.1 to about 1.0%.

Suitable sweeteners include aspartame and saccharin. The amount of sweetener(s) in a systemic or topical composition is typically about 0.001 to about 1%.

Suitable antioxidants include butylated hydroxyanisole (“BHA”), butylated hydroxytoluene (“BHT”), and vitamin E. The amount of antioxidant(s) in a systemic or topical composition is typically about 0.1 to about 5%.

Suitable preservatives include benzalkonium chloride, methyl paraben and sodium benzoate. The amount of preservative(s) in a systemic or topical composition is typically about 0.01 to about 5%.

Suitable glidants include silicon dioxide. The amount of glidant(s) in a systemic or topical composition is typically about 1 to about 5%.

Suitable solvents include water, isotonic saline, ethyl oleate, glycerine, hydroxylated castor oils, alcohols such as ethanol, dimethyl sulfoxide, N-methyl-2-pyrrolidone, dimethylacetamide and phosphate (or other suitable buffer). The amount of solvent(s) in a systemic or topical composition is typically from about 0 to about 100%.

Suitable suspending agents include AVICEL RC-591 (from FMC Corporation of Philadelphia, Pa.) and sodium alginate. The amount of suspending agent(s) in a systemic or topical composition is typically about 1 to about 8%.

Suitable surfactants include lecithin, Polysorbate 80, and sodium lauryl sulfate, and the TWEENS from Atlas Powder Company of Wilmington, Del. Suitable surfactants include those disclosed in the C.T.F.A. Cosmetic Ingredient Handbook, 1992, pp. 587-592; Remington's Pharmaceutical Sciences, 15th Ed. 1975, pp. 335-337; and McCutcheon's Volume 1, Emulsifiers & Detergents, 1994, North American Edition, pp. 236-239. The amount of surfactant(s) in the systemic or topical composition is typically about 0.1% to about 5%.

Suitable cyclodextrins include alpha-CD, beta-CD, gamma-CD, hydroxypropyl betadex (HP-beta-CD), sulfobutyl-ether β-cyclodextrin (SBE-beta-CD). The amount of cyclodextrins in the systemic or topical composition is typically about 0% to about 40%.

Although the amounts of components in the systemic compositions may vary depending on the type of systemic composition prepared, in general, systemic compositions include 0.01% to 50% of an active compound (e.g., a compound of formula (I)) and 50% to 99.99% of one or more carriers. Compositions for parenteral administration typically include 0.1% to 10% of actives and 90% to 99.9% of a carrier including a diluent and a solvent.

Compositions for oral administration can have various dosage forms. For example, solid forms include tablets, capsules, granules, and bulk powders. These oral dosage forms include a safe and effective amount, usually at least about 5%, and more particularly from about 25% to about 50% of actives. The oral dosage compositions include about 50% to about 95% of carriers, and more particularly, from about 50% to about 75%.

Tablets can be compressed, tablet triturates, enteric-coated, sugar-coated, film-coated, or multiple-compressed. Tablets typically include an active component, and a carrier comprising ingredients selected from diluents, lubricants, binders, disintegrants, colorants, flavors, sweeteners, glidants, and combinations thereof. Specific diluents include calcium carbonate, sodium carbonate, mannitol, lactose, and cellulose. Specific binders include starch, gelatin, and sucrose. Specific disintegrants include alginic acid and croscarmellose. Specific lubricants include magnesium stearate, stearic acid, and tale. Specific colorants are the FD&C dyes, which can be added for appearance. Chewable tablets preferably contain sweeteners such as aspartame and saccharin, or flavors such as menthol, peppermint, fruit flavors, or a combination thereof.

Capsules (including implants, time release and sustained release formulations) typically include an active compound (e.g., a compound of formula (I)), and a carrier including one or more diluents disclosed above in a capsule comprising gelatin. Granules typically comprise a disclosed compound, and preferably glidants such as silicon dioxide to improve flow characteristics. Implants can be of the biodegradable or the non-biodegradable type.

The selection of ingredients in the carrier for oral compositions depends on secondary considerations like taste, cost, and shelf stability, which are not critical for the purposes of this invention.

Solid compositions may be coated by conventional methods, typically with pH or time-dependent coatings, such that a disclosed compound is released in the gastrointestinal tract in the vicinity of the desired application, or at various points and times to extend the desired action. The coatings typically include one or more components selected from the group consisting of cellulose acetate phthalate, polyvinyl acetate phthalate, hydroxypropyl methyl cellulose phthalate, ethyl cellulose, EUDRAGIT® coatings (available from Evonik Industries of Essen, Germany), waxes and shellac.

Compositions for oral administration can have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically include a disclosed compound and a carrier, namely, a carrier selected from diluents, colorants, flavors, sweeteners, preservatives, solvents, suspending agents, and surfactants. Peroral liquid compositions preferably include one or more ingredients selected from colorants, flavors, and sweeteners.

Other compositions useful for attaining systemic delivery of the subject compounds include sublingual, buccal and nasal dosage forms. Such compositions typically include one or more of soluble filler substances such as diluents including sucrose, sorbitol, and mannitol; and binders such as acacia, microcrystalline cellulose, carboxymethyl cellulose, and hydroxypropyl methylcellulose. Such compositions may further include lubricants, colorants, flavors, sweeteners, antioxidants, and glidants.

The disclosed compounds can be topically administered. Topical compositions that can be applied locally to the skin may be in any form including solids, solutions, oils, creams, ointments, gels, lotions, shampoos, leave-on and rinse-out hair conditioners, milks, cleansers, moisturizers, sprays, skin patches, and the like. Topical compositions include: a disclosed compound (e.g., a compound of formula (I)), and a carrier. The carrier of the topical composition preferably aids penetration of the compounds into the skin. The carrier may further include one or more optional components.

The amount of the carrier employed in conjunction with a disclosed compound is sufficient to provide a practical quantity of composition for administration per unit dose of the compound. Techniques and compositions for making dosage forms useful in the methods of this invention are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2nd Ed., (1976).

A carrier may include a single ingredient or a combination of two or more ingredients. In the topical compositions, the carrier includes a topical carrier. Suitable topical carriers include one or more ingredients selected from phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof, and the like. More particularly, carriers for skin applications include propylene glycol, dimethyl isosorbide, and water, and even more particularly, phosphate buffered saline, isotonic water, deionized water, monofunctional alcohols, and symmetrical alcohols.

The carrier of a topical composition may further include one or more ingredients selected from emollients, propellants, solvents, humectants, thickeners, powders, fragrances, pigments, and preservatives, all of which are optional.

Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, tricthylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin include stearyl alcohol and polydimethylsiloxane. The amount of emollient(s) in a skin-based topical composition is typically about 5% to about 95%.

Suitable propellants include propane, butane, isobutane, dimethyl ether, carbon dioxide, nitrous oxide, and combinations thereof. The amount of propellant(s) in a topical composition is typically about 0% to about 95%.

Suitable solvents include water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof. Specific solvents include ethyl alcohol and homotopic alcohols. The amount of solvent(s) in a topical composition is typically about 0% to about 95%.

Suitable humectants include glycerin, sorbitol, sodium 2-pyrrolidone-5-carboxylate, soluble collagen, dibutyl phthalate, gelatin, and combinations thereof. Specific humectants include glycerin. The amount of humectant(s) in a topical composition is typically 0% to 95%.

The amount of thickener(s) in a topical composition is typically about 0% to about 95%.

Suitable powders include beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemically-modified magnesium aluminum silicate, organically-modified montmorillonite clay, hydrated aluminum silicate, fumed silica, carboxyvinyl polymer, sodium carboxymethyl cellulose, ethylene glycol monostearate, and combinations thereof. The amount of powder(s) in a topical composition is typically 0% to 95%.

The amount of fragrance in a topical composition is typically about 0% to about 0.5%, particularly, about 0.001% to about 0.1%.

Suitable pH adjusting additives include HCl or NaOH in amounts sufficient to adjust the pH of a topical pharmaceutical composition.

b. Albumin Compositions

The disclosure further provides compositions comprising albumin nanoparticles. In some embodiments, a compound as disclosed herein is incorporated into compositions comprising an albumin nanoparticle. The albumin nanoparticle compositions and formulations may also include pharmaceutically acceptable carriers, as described above.

Albumins include the most abundant plasma proteins in mammals and albumins from a large and diverse number of mammals have been characterized by biochemical methods and/or by sequence information. Any natural, synthetic, or engineered albumin may be used in the context of the nanoparticle compositions described herein. In some embodiments, the albumin is human serum albumin.

In some embodiments, the albumin nanoparticles further comprise one or more cell targeting epitopes. In some embodiments, the epitopes are covalently attached or directly conjugated to the albumin. In some embodiments, the epitopes are crosslinked to the albumin. In select embodiments, the albumin nanoparticles further comprise one or more immune cell epitopes (e.g., B cell and T cell epitopes). The one or more immune cell antigens may facilitate targeting to lymphatic systems. In select embodiments, the albumin nanoparticles further comprise one or more epitopes from a microbiological agent (e.g., Clostridioides difficile, Bacillus anthracis, Clostridium botulinum, Heliobacter pylori, Rotavirus sp., Coronaviridae).

c. Lipophilic Formulations

In some embodiments, the compounds disclosed herein are incorporated into lipophilic compositions comprising a liposome, a lipid nanoparticle, a micelle, or the like. In some embodiments, a disclosed compound is encapsulated in the liposome, the lipid nanoparticle, or the micelle. The formulations may also include pharmaceutically acceptable carriers, as described above.

In some embodiments, the disclosed compounds are incorporated into lipophilic compositions comprising one or more vesicle forming lipids. Methods of making lipophilic compositions include, for example, lipid film hydration, optionally coupled with sonication or extrusion, solvent evaporation (e.g., ethanol injection, ether injection, or reverse phase evaporation), solvent-diffusion method, hot homogenization process, detergent removal methods, or combinations thereof. The disclosed compounds can be combined with the lipid(s) before formation of the vesicles (passive loading) or after vesicle formation (active loading). The lipophilic compositions may prolong circulation time in vivo, increase stability of the compound, and prevent degradation in the bloodstream. The lipophilic compositions may increase the distribution of the compounds within the lung, breast, pancreas, and spleen.

Any naturally occurring or synthetic vesicle forming lipid or combinations thereof can be used. The one or more vesicle forming lipids may be selected from di-aliphatic chain lipids, such as phospholipids; diglycerides; di-aliphatic glycolipids; single lipids such as sphingomyelin or glycosphingolipid; steroidal lipids; hydrophilic polymer derivatized lipids; or mixtures thereof.

Lipophilic compositions of the disclosure may include one or more cationic and/or ionizable lipids, phospholipids, neutral or non-cationic lipids, polyethylene glycol (PEG)-lipid conjugates, and/or sterols. In some embodiments, the lipid nanoparticle comprises a cationic lipid and/or ionizable lipid, a neutral or non-cationic lipid, and cholesterol.

Cationic and/or ionizable lipids include, for example, amine-containing lipids that can be readily protonated and may have a positive or partial positive charge at physiological pH due to a pKa value between pH 5 and 8. The polar headgroup of the cationic lipids preferably comprises amine derivatives such as primary, secondary, and/or tertiary amines, quaternary ammonium, various combinations of amines, amidium salts, or guanidine and/or imidazole groups as well as pyridinium, piperazine and amino acid headgroups such as lysine, arginine, ornithine and/or tryptophan. Cationic lipids include, but are not limited to, 1,2-dimyristoyl-sn-glycero-3-ethylphosphocholine (DMEPC), 1,2-di-O-octadecenyl-3-trimethylammonium propane (DOTMA) and/or 1,2-dioleoyl-3-trimethylammonium propane (DOTAP), 1,2-dimyristoyl-3-trimethylammonium propane (DMTAP), 2,3-di (tetradecoxy) propyl-(2-hydroxyethyl)-dimethylazanium bromide (DMRIE), didodecyl (dimethyl) ammonium bromide (DDAB), 1,2-dioleyloxypropyl-3-dimethyl-hydroxyethyl ammonium bromide (DORIE), 3β-[N—(N\N′-dimethylamino-ethane) carbamoyl]cholesterol (DC-Chol) or dioleyl ether phosphatidylcholine (DOEPC). Ionizable lipids include, but are not limited to, 1,2-dioleyloxy-3-dimethylamino-propane (DODMA).

In some embodiments, the lipophilic compositions comprise a polyethylene glycol (PEG)-lipid conjugate. A PEG-lipid conjugate may include, but is not limited to, PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-DMG (1,2-dimyristoyl-rac-glycero-3-methoxypolyethylene glycol), PEG-c-DOMG (R-3-[(ω-methoxy poly (ethylene glycol)2000) carbamoyl)]-1,2-dimyristyloxlpropyl-3-amine), PEG-DMA (PEG-dimethacrylate), PEG-DLPE (1,2-didodecanoyl-sn-glycero-3-phosphoethanolamine-PEG), PEG-DMPE (PEG-1,2-dimyristoyl-sn-glycero-3-phosphoethanolamine), PEG-DPPC (PEG-dipalmitoyl phosphatidylcholine), PEG-N,N-di (tetradecyl) acetamide, or a PEG-DSPE (1, 2-distearoyl-sn-glycero-3-phosphoethanolamine-poly (ethylene glycol)) lipid. In some embodiments, the lipid nanoparticle comprises PEG-DMG and/or PEG-N,N-di (tetradecyl) acetamide.

The sterol may comprise cholesterol, fecosterol, ergosterol, campesterol, sitosterol, stigmasterol, brassicasterol, or a sterol ester, such as cholesteryl hemisuccinate, cholesteryl sulfate, or any other derivatives of cholesterol.

A neutral or non-cationic lipid may include one or more phospholipids. Phospholipids include a phospholipid moiety and one or more fatty acid moieties. A phospholipid moiety may include, but is not limited to, phosphatidyl choline, phosphatidyl ethanolamine, phosphatidyl glycerol, phosphatidyl serine, phosphatidic acid, 2-lysophosphatidyl choline, and sphingomyelin. A fatty acid moiety may include, but is not limited to, lauric acid, myristic acid, myristoleic acid, palmitic acid, palmitoleic acid, stearic acid, oleic acid, linoleic acid, alpha-linolenic acid, erucic acid, phytanic acid, arachidic acid, arachidonic acid, eicosapentaenoic acid, behenic acid, docosapentaenoic acid, and docosahexaenoic acid.

Phospholipids suitable for use in the compositions may include, but are not limited to, phosphatidylglycerol (PG) including dimyristoyl phosphatidylglycerol (DMPG) and 1,2-dioleoyl-sn-glycero-3-phospho-rac-(1-glycerol) sodium salt (DOPG); phosphatidylcholine (PC), including egg yolk phosphatidylcholine, dimyristoyl phosphatidylcholine (DMPC), 1,2-distearoyl-sn-glycero-3-phosphocholine (DSPC), 1,2-dilinoleoyl-sn-glycero-3-phosphocholine (DLPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-didocosahexaenoyl-sn-glycero-3-phosphocholine, 1,2-diarachidonoyl-sn-glycero-3-phosphocholine; phosphatidylethanolamine (PE) including 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine; phosphatidic acid (PA); phosphatidylinositol (PI); phosphatidylserine (PS); and sphingomyelin (SM).

The positively charged lipid structures described herein may also include other components typically used in the formation of vesicles (e.g., for stabilization). Examples of such other components includes, without being limited thereto, fatty alcohols, fatty acids, and/or any other pharmaceutically acceptable excipients which may affect the surface charge, the membrane fluidity and assist in the incorporation of the lipid into the lipid assembly.

The lipophilic compositions can also be targeting, e.g., contain one or more targeting moieties or biodistribution modifiers on the surface. A targeting moiety can be any agent that is capable of specifically binding or interacting with a desired target and are generally known in the art, for example ligands such as folic acid, proteins, antibody or antibody fragments, and the like). In some embodiments, the targeting moiety is an immune cell epitope (e.g., B cell and T cell epitopes). In select embodiments, the targeting moiety comprises one or more epitopes from a microbiological agent (e.g., Clostridioides difficile, Bacillus anthracis, Clostridium botulinum, Heliobacter pylori, Rotavirus sp., Coronaviridae).

The lipophilic compositions can have any structure, e.g., structures having an inner space sequestered from the outer medium by one or more lipid bilayers, or any microcapsule that has a semi-permeable membrane with a lipophilic central part where the membrane sequesters an interior. In some embodiments, the lipophilic compositions may comprise unilamellar liposomes, having a single lipid layer. The disclosed compounds may be completely or partially located in the interior space of the liposome or completely or partially within the bilayer membrane of the liposome. In some embodiments, the lipophilic compositions comprise micelles.

d. Additional Formulations

In some embodiments, the disclosed compounds are incorporated into formulations comprising PLA and/or PLGA. PLA or PLGA formulations may be prepared by various methods known in the art such as single/double emulsion-solvent evaporation technique, spray drying, spray freeze drying, supercritical fluid drying, and nanoprecipitation.

In some embodiments, the disclosed compounds are incorporated into polymeric drug delivery systems formed by polymers of naturally occurring materials (e.g., poly-arginine, chitosan, dextrin, polysaccharides, poly (glycolic acid), poly(lactic acid), and hyaluronic acid) or synthetic polymers (e.g., poly (2-hydroxyethyl methacrylate), poly (N-isopropyl acrylamide) s, poly (ethylenimine) s, dendritic polymers, and the like). Formulations may be prepared by various methods known in the art such as a single/double emulsion-solvent evaporation technique, spray drying, spray freeze drying, supercritical fluid drying, and nanoprecipitation.

In some embodiments, the disclosed compounds are incorporated into an organic nanoparticles formed by a peptide, protein, nucleic acid, or any combination thereof.

In some embodiments, the disclosed compounds are incorporated into an inorganic nanoparticle formed by silica, gold, silver, iron, or the like.

e. Additional Therapeutic Agents

Any of the above compositions or formulations disclosed herein may further comprise at least one additional therapeutic agent. In some embodiments the at least one additional therapeutic agent comprises an immune modulator, a chemotherapeutic agent, a nucleic acid (e.g., mRNA, aptamers, antisense oligonucleotides, ribozyme nucleic acids, interfering RNAs, antisense and antigene nucleic acids), a decongestant, a steroid, an analgesic, an antimicrobial agent, an immunotherapy, or a combination thereof. In some embodiments, the at least one additional therapeutic agent is selected from a chemotherapeutic agent, an indoleamine 2,3-dioxygenase (IDO) inhibitor, a Stat3 inhibitor, a TLR agonist, PD-1 or PD-L1 antibody, and a phosphatidylinositol 3-kinase (PI3K) inhibitor (e.g., a Class I PI3K inhibitors, an isoform-selective PI3K inhibitor).

Exemplary immune modulators include: indoleamine 2,3-dioxygenase (IDO) inhibitors and analogs thereof, such as, epacadostat, BMS-986205, indoximod, PF-06840003, and analogs thereof; signal transducer and activator of transcription 3 (Stat3) inhibitors and analogs thereof, such as, SM-36 and its analogs; toll-like receptor (TLR) agonists and analogs thereof, such as, imiquimod, resiquimod, selgantolimod, gardiquimod, SM-360320, TMX-101, TMX-202, TMX-302, TMX-306, GSK2245035, CL097, 852A, AZD-8848, DSP-3025, GS-9620, RO7020531, RO6871765, ANA773, DSP-0509, NJH395, BNT411, TQ-A3334, JNJ-4964, LHC165, CV8102, VTX-1463, VTX-2337, IMO-8400, IMO-3100, IRS-954, and analogs thereof; and statins or other lipid-lowering medications and analogs thereof, such as, atorvastatin, pravastatin, fluvastatin, simvastatin, lovastatin, mevastatin, pitavastatin, rosuvastatin, and analogs thereof.

In some embodiments, the at least one additional therapeutic agent comprises at least one chemotherapeutic agent. As used herein, the term “chemotherapeutic” or “anti-cancer drug” includes any small molecule or other drug used in cancer treatment or prevention. Chemotherapeutics include, but are not limited to, cyclophosphamide, methotrexate, 5-fluorouracil, doxorubicin, docetaxel, daunorubicin, bleomycin, vinblastine, dacarbazine, cisplatin, paclitaxel, raloxifene hydrochloride, tamoxifen citrate, abemacicilib, afinitor (Everolimus), alpelisib, anastrozole, pamidronate, anastrozole, exemestane, capecitabine, epirubicin hydrochloride, eribulin mesylate, toremifene, fulvestrant, letrozole, gemcitabine, goserelin, ixabepilone, emtansine, lapatinib, olaparib, megestrol, neratinib, palbociclib, ribociclib, talazoparib, thiotepa, toremifene, methotrexate, and tucatinib.

In some embodiments, the at least one additional therapeutic agent comprises a polynucleotide or nucleic acid (e.g., ribonucleic acid or deoxyribonucleic acid). The term “polynucleotide,” in its broadest sense, includes any compound and/or substance that is or can be incorporated into an oligonucleotide chain. Exemplary polynucleotides for use in accordance with the present disclosure include, but are not limited to, one or more of deoxyribonucleic acid (DNA), ribonucleic acid (RNA) including messenger mRNA (mRNA), hybrids thereof, RNAi-inducing agents, RNAi agents, siRNAs, shRNAs, miRNAs, antisense RNAs, ribozymes, catalytic DNA, RNAs that induce triple helix formation, aptamers, vectors, etc.

In some embodiments, the at least one additional therapeutic agent is an RNA. RNAs useful in the compositions and methods described herein can be selected from the group consisting of, but are not limited to, shortmers, antagomirs, antisense RNAs, ribozymes, small interfering RNA (siRNA), asymmetrical interfering RNA (aiRNA), microRNA (miRNA), Dicer-substrate RNA (dsRNA), small hairpin RNA (shRNA), transfer RNA ((RNA), messenger RNA (mRNA), and mixtures thereof.

In certain embodiments, the at least one additional therapeutic agent is an mRNA. An mRNA may encode any polypeptide of interest, including any naturally or non-naturally occurring or otherwise modified polypeptide. A polypeptide encoded by an mRNA may be of any size and may have any secondary structure or activity. In some embodiments, a polypeptide encoded by an mRNA may have a therapeutic effect when expressed in a cell.

In other embodiments, the at least one additional therapeutic agent is an siRNA. An siRNA may be capable of selectively knocking down or down regulating expression of a gene of interest. For example, an siRNA could be selected to silence a gene associated with a particular disease, disorder, or condition upon administration to a subject in need thereof of a nanoparticle composition including the siRNA. An siRNA may comprise a sequence that is complementary to an mRNA sequence that encodes a gene or protein of interest. In some embodiments, the siRNA may be an immunomodulatory siRNA.

In some embodiments, the at least one additional therapeutic agent is an shRNA or a vector or plasmid encoding the same. An shRNA may be produced inside a target cell upon delivery of an appropriate construct to the nucleus. Constructs and mechanisms relating to shRNA are well known in the relevant arts.

The PI3K inhibitors may target any class of PI3K, including Class I (e.g., IA and IB), Class II, or Class III. In some embodiments, the PI3K inhibitors is a compound as disclosed herein. In some embodiments, the PI3K inhibitors comprises isoform-selective PI3K inhibitors, dual pan-Class I PI3K/m-TOR inhibitors, and pan-Class I PI3K inhibitors without significant m-TOR activity. PI3K inhibitors useful in the present compositions and methods include, but are not limited to, IPI-549, idelalisib, copanlisib, duvelisib, alpelisib, leniolisib, umbralisib, buparlisib, taselisib, pictilisib, PX-886, pilaralisib, BEZ235, GSK2126458, GSK2636771, AZD8186, SAR260301, gedatolisib, apitolisib, PQR309, MLN1117, and perifosine.

f. Vaccines

The compounds and compositions may also be used for vaccines. The vaccines comprise the compound or compositions disclosed and an antigen or a nucleic acid encoding thereof. Suitable antigens include microbial pathogens, bacteria, viruses, proteins, glycoproteins lipoproteins, peptides, glycopeptides, lipopeptides, toxoids, carbohydrates, and tumor-specific antigens. Mixtures of two or more antigens may be employed.

The antigen can be derived and/or isolated from essentially any desired source depending on the infectious disease, autoimmune disease, condition, cancer, pathogen, or a disease that is to be treated with a given vaccine composition.

The vaccines described herein may be capable of providing immunity against one or more conditions related to infectious diseases, including but not limited to, influenza, measles, human papillomavirus (HPV), rabies, meningitis, whooping cough, tetanus, plague, hepatitis, and tuberculosis and can include infectious disease derived antigens and/or epitopes, or nucleic acids encoding thereof.

The vaccines described herein may also direct an immune response against cancer cells and can include tumor cell derived antigens, epitopes, and/or neoepitopes, or portions thereof, or nucleic acids encoding tumor cell derived antigens, epitopes, and/or neoepitopes. Tumor antigens are surface molecules that are differentially expressed in tumor cells relative to non-tumor tissues. Tumor antigens make tumor cells immunologically distinct from normal cells and provide diagnostic and therapeutic targets for human cancers. Tumor antigens have been characterized either as membrane proteins or as altered carbohydrate molecules of glycoproteins or glycolipids on the cell surface. Cancer cells often have distinctive tumor antigens on their surfaces, such as truncated epidermal growth factor, folate binding protein, epithelial mucins, melanoferrin, carcinoembryonic antigen, prostate-specific membrane antigen, HER2-neu, which are candidates for use in therapeutic cancer vaccines. Because tumor antigens are normal or related to normal components of the body, the immune system often fails to mount an effective immune response against those antigens to destroy the tumor cells. Illustrative cancer types for which this approach can be used include prostate, colon, breast, ovarian, pancreatic, brain, bead and neck, melanoma, leukemia, lymphoma, etc.

In other embodiments, the antigen present in the vaccine composition is not a foreign antigen, but a self-antigen, e.g., the vaccine composition is directed toward an autoimmune disease. Examples of autoimmune diseases include type 1 diabetes, conventional organ specific autoimmunity, neurological disease, rheumatic diseases/connective tissue disease, autoimmune cytopenias, and related autoimmune diseases. Such conventional organ specific autoimmunity may include thyroiditis (Graves+Hashimoto's), gastritis, adrenalitis (Addison's), ovaritis, primary biliary cirrhosis, myasthenia gravis, gonadal failure, hypoparathyroidism, alopecia, malabsorption syndrome, pernicious anemia, hepatitis, anti-receptor antibody diseases and vitiligo. Such neurological diseases may include schizophrenia, Alzheimer's disease, depression, hypopituitarism, diabetes insipidus, sicca syndrome and multiple sclerosis. Such rheumatic diseases/connective tissue diseases may include rheumatoid arthritis, systemic lupus erythematous (SLE) or Lupus, scleroderma, polymyositis, inflammatory bowel disease, dermatomyositis, ulcerative colitis, Crohn's disease, vasculitis, psoriatic arthritis, exfoliative psoriatic dermatitis, pemphigus vulgaris, Sjogren's syndrome. Other autoimmune related diseases may include autoimmune uvoretinitis, glomerulonephritis, post myocardial infarction cardiotomy syndrome, pulmonary hemosiderosis, amyloidosis, sarcoidosis, aphthous stomatitis, and other immune related diseases, as presented herein and known in the related arts.

In one embodiment, the antigen in a vaccine composition is a peptide, polypeptide, or immunogenic portion thereof. An “immunogenic portion,” as used herein is a portion of a protein that is recognized (e.g., specifically bound) by a B cell and/or T cell surface antigen receptor. Such immunogenic portions generally comprise at least 5 amino acid residues, more preferably at least 10, and still more preferably at least 20 amino acid residues of an antigenic protein or a variant thereof.

Immunogenic portions of antigen polypeptides may generally be identified using well known techniques, such as those summarized in Paul, Fundamental Immunology, 3rd ed., 243-247 (Raven Press, 1993) and references cited therein. Such techniques include screening polypeptides for the ability to react with antigen-specific antibodies, antisera and/or T cell lines or clones. As used herein, antisera and antibodies are “antigen-specific” if they specifically bind to an antigen (e.g., they react with the protein in an ELISA or other immunoassay, and do not react detectably with unrelated proteins). Such antisera and antibodies may be prepared using known techniques. An immunogenic portion of a protein is a portion that reacts with such antisera and/or T cells at a level that is not substantially less than the reactivity of the full length polypeptide (e.g., in an ELISA and/or T cell reactivity assay). Such immunogenic portions may react within such assays at a level that is similar to or greater than the reactivity of the full length polypeptide. Such screens may generally be performed using methods well known to those of ordinary skill in the art, such as those described in Harlow and Lane, Antibodies: A Laboratory Manual, Cold Spring Harbor Laboratory, 1988. For example, a polypeptide may be immobilized on a solid support and contacted with patient sera to allow binding of antibodies within the sera to the immobilized polypeptide. Unbound sera may then be removed and bound antibodies detected using, for example, ¹²⁵I-labeled Protein A.

Peptide and polypeptide antigens may be prepared using any of a variety of well-known techniques. Recombinant polypeptides encoded by DNA sequences may be readily prepared from isolated DNA sequences using any of a variety of expression vectors known to those of ordinary skill in the art. Expression may be achieved in any appropriate host cell that has been transformed or transfected with an expression vector containing a DNA molecule that encodes a recombinant polypeptide. Suitable host cells include prokaryotes, yeast, and higher eukaryotic cells, such as mammalian cells and plant cells. Preferably, the host cells employed are E. coli, yeast, or a mammalian cell line such as COS or CHO.

Portions and other variants of a protein antigen having less than about 100 amino acids, and generally less than about 50 amino acids, may also be generated by synthetic means, using techniques well known to those of ordinary skill in the art. For example, such polypeptides may be synthesized using any of the commercially available solid-phase techniques, such as the Merrifield solid-phase synthesis method, where amino acids are sequentially added to a growing amino acid chain. See, Merrifield, J. Am. Chem. Soc. 85:2149-2146, 1963. Equipment for automated synthesis of polypeptides is commercially available from suppliers such as Perkin Elmer/Applied BioSystems Division (Foster City, Calif.), and may be operated according to the manufacturer's instructions.

In certain embodiments, the nucleic acid encoding the antigen is DNA. Illustrative DNA-based vaccines of this type contain DNA encoding one or more polypeptide antigens, such that the antigen is generated in situ. Alternatively, the vaccine may be an RNA-based vaccine. In certain embodiments, the nucleic acid encoding the antigen is an mRNA. An mRNA may encode any polypeptide antigen of interest, including any naturally or non-naturally occurring or otherwise modified polypeptide. A polypeptide encoded by an mRNA may be of any size and may have any secondary structure or activity. In some embodiments, a polypeptide encoded by the mRNA may stimulate an immune response when expressed in a cell.

The vaccine compositions of the present disclosure may also contain other compounds, which may be biologically active or inactive. The vaccine or medicament may comprise an adjuvant or immunostimulant, or a polynucleotide encoding an adjuvant or immunostimulant (e.g., an adjuvantive polypeptide). Adjuvants and immunostimulants are compounds or compositions that either directly or indirectly stimulate the immune system's response to a co-administered antigen. In some embodiments, the vaccines are not adjuvanted or are self-adjuvanting.

Suitable adjuvants are commercially available as, for example, Glucopyranosyl Lipid Adjuvant (GLA); Pam3CSK4; Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham); mineral salts (for example, aluminum, silica, kaolin, and carbon); aluminum salts such as aluminum hydroxide gel (alum), AIK (SO₄)₂, AlNa(SO₄)₂, AlNHA (SO₄), and AI (OH)₃; salts of calcium (e.g., Ca₃(PO₄)₂), iron or zinc; an insoluble suspension of acylated tyrosine; acylated sugars; cationically or anionically derivatized polysaccharides; polynucleotides (for example, poly IC, poly AU acids, and CpG oligodeoxynucleotides (e.g., Class A or B)); polyphosphazenes; cyanoacrylates; polymerase-(DL-lactide-co-glycoside); bovine serum albumin; diphtheria toxoid; tetanus toxoid; edestin; keyhole-limpet hemocyanin; Pseudomonal Toxin A; choleragenoid; cholera toxin; pertussis toxin; viral proteins; Quil A; aminoalkyl glucosamine phosphate compounds. In addition, adjuvants such as cytokines (e.g., GM-CSF or interleukin-2, -7, or -12), interferons, or tumor necrosis factor, may also be used as adjuvants. Protein and polypeptide adjuvants may be obtained from natural or recombinant sources according to methods well known to those skilled in the art. When obtained from recombinant sources, the adjuvant may comprise a protein fragment comprising at least the immunostimulatory portion of the molecule.

Other known immunostimulatory macromolecules which can be used include, but are not limited to, polysaccharides, tRNA, non-metabolizable synthetic polymers such as polyvinylamine, polymethacrylic acid, polyvinylpyrrolidone, mixed polycondensates (with relatively high molecular weight) of 4′,4-diaminodiphenylmethane-3,3′-dicarboxylic acid and 4-nitro-2-aminobenzoic acid (Sec, Sela, M., Science 166:1365-1374 (1969)) or glycolipids, lipids or carbohydrates.

In some embodiments, the adjuvantive polypeptide comprises immune activator proteins, such as CD70, CD40 ligand, and constitutively active TLR4, or polycationic peptides (e.g., protamine). In some embodiments, the adjuvantive polypeptide is a flagellin polypeptide. Commercially available mRNA encoding adjuvantive polypeptides are available, for example, as TriMix (See Bonehill, A. et al. Mol. Ther. 16, 1170-1180 (2008), incorporated herein by reference). In some embodiments, the vaccine may comprise at least two separate polynucleotides, one encoding anti-Müllerian hormone receptor II extracellular domain (AMHR2-ED), as described above, and the other encoding an adjuvantive polypeptide (e.g., a flagellin polypeptide or immune activator protein).

Vaccine preparation is a well-developed art and general guidance in the preparation and formulation of vaccines is readily available from any of a variety of sources. One such example is New Trends and Developments in Vaccines, edited by Volier et al. University Park Press, Baltimore, Md., U.S.A. 1978. Vaccine compositions may generally be used for prophylactic and therapeutic purposes.

The amount of antigen in each vaccine dose is generally selected as an amount which induces an immunoprotective response without significant adverse side effects in typical vaccines. Such amount will vary depending upon which specific immunogen is employed and how it is presented. Of course, the dosage administered may be dependent upon the age, weight, kind of concurrent treatment, if any, and nature of the antigen administered.

The immunogenic activity of a given amount of a vaccine composition can be readily determined, for example by monitoring the increase in titer of antibody against the antigen used in the vaccine composition (Dalsgaard, K. Acta Veterinia Scandinavica 69:1-40 (1978)). Another common method involves injecting CD-1 mice intradermally with various amounts of a vaccine composition, later harvesting sera from the mice and testing for anti-immunogen antibody, e.g., by ELISA. These and other similar approaches will be apparent to the skilled artisan.

### 4. METHODS OF USE

The disclosure provides methods for inducing or modulating an immune or inflammatory response. As used herein, the term “modulating” generally refers to the ability to alter, by increasing or decreasing, e.g., directly or indirectly promoting/stimulating/up-regulating or interfering with/inhibiting/down-regulating a specific concentration, level, expression, function or behavior (e.g., of the immune or inflammatory response). In some embodiments, the modulating is an increase and/or decrease of a certain concentration, level, activity, or function relative to a control, or relative to the average level of activity that would generally be expected or relative to a control level of activity.

Thus, in some embodiments, modulating an immune or inflammatory response refers to the ability of the compounds of the present invention to alter or modulate one or more aspects of the immune or inflammatory response. In some embodiments, the methods polarize macrophages. In some embodiments, the methods induce an interferon response. In some embodiments, the methods active transcription factors (e.g., STAT6, IRF3) of the innate immune response.

The disclosure further provides methods for treating a disease or disorder comprising administration of a compound or composition as disclosed herein, to a subject in need thereof. In some embodiments, the subject is a human.

The disease or disorder may comprise cancer, autoimmune diseases, inflammatory diseases, and infectious diseases.

In some embodiments, the disease or disorder is an inflammatory disease or disorder. Inflammatory diseases are characterized by activation of the immune system in a tissue or an organ to abnormal levels that may lead to abnormal function and/or disease in the tissue or organ. The inflammatory diseases and disorders that may be treated by the methods of the present invention include, but are not limited to, arthritis, rheumatoid arthritis, asthma, inflammatory bowel disease (Crohn's disease or ulcerative colitis), chronic obstructive pulmonary disease (COPD), allergic rhinitis, vasculitis (polyarteritis nodosa, temporal arteritis, Wegener's granulomatosis, Takayasu's arteritis, or Behcet's syndrome), inflammatory neuropathy, psoriasis, systemic lupus erythematosus (SLE), chronic thyroiditis, Hashimoto's thyroiditis, Addison's disease, polymyalgia rheumatica, Sjogren's syndrome, or Churg-Strauss syndrome.

In some embodiments, the disease or disorder is an autoimmune disease or disorder. Autoimmune diseases and disorders refer to conditions in a subject characterized by cellular, tissue and/or organ injury caused by an immunologic reaction of the subject to its own cells, tissues and/or organs. Autoimmune diseases and disorders that may be treated by the methods of the present invention include, but are not limited to, alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatricial pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), irritable bowel disease (IBD), IgA neuropathy, juvenile arthritis, lichen planus, lupus erythematosus, Meniere's disease, mixed connective tissue disease, multiple sclerosis, type 1 or immune-mediated diabetes mellitus, myasthenia gravis, pemphigus vulgaris, pernicious anemia, polyarteritis nodosa, polychondritis, polyglandular syndromes, polymyalgia rheumatics, polymyositis and dermatomyositis, primary agammaglobulinemia, primary biliary cirrhosis, psoriasis, psoriatic arthritis, Raynaud's phenomenon, Reiter's syndrome, Rheumatoid arthritis, sarcoidosis, scleroderma, Sjogren's syndrome, stiff-man syndrome, systemic lupus erythematosus, lupus erythematosus, takayasu arteritis, temporal arteritis/giant cell arteritis, ulcerative colitis, uveitis, vasculitides such as dermatitis berpetiformis vasculitis, vitiligo, and Wegener's granulomatosis.

Some autoimmune disorders are also associated with an inflammatory condition. Examples of inflammatory disorders which are also autoimmune disorders that can be prevented, treated or managed in accordance with the methods of the invention include, but are not limited to, asthma, encephalitis, inflammatory bowel disease, chronic obstructive pulmonary disease (COPD), allergic disorders, pulmonary fibrosis, undifferentiated spondyloarthropathy, undifferentiated arthropathy, arthritis, inflammatory osteolysis, and chronic inflammation resulting from chronic viral or bacterial infections. Examples of the types of psoriasis which can be treated in accordance with the compositions and methods of the invention include, but are not limited to, plaque psoriasis, pustular psoriasis, erythrodermic psoriasis, guttate psoriasis and inverse psoriasis. Some autoimmune disorders are also associated with immune dysregulatory diseases including, for example, sinopulmonary infections, opportunistic pneumonias, inflammatory bowel disease, autoimmune hepatitis and juvenile idiopathic arthritis, and myelofibrosis.

In some embodiments, the disease or disorder is cancer. In some embodiments, the cancer comprises a solid tumor. In some embodiments, the cancer comprises a blood cancer or lymphoma. In some embodiments, the cancer is metastatic cancer. In some embodiments, the disclosed compounds, compositions, or methods result in suppression of elimination of metastasis. In some embodiments, the disclosed compounds, compositions, or methods result in decreased tumor growth. In some embodiments, the disclosed compounds, compositions, or methods prevent tumor recurrence.

The compounds and compositions herein may be useful to treat a wide variety of cancers including carcinoma, sarcoma, lymphoma, leukemia, melanoma, mesothelioma, multiple myeloma, or seminoma. The cancer may be a cancer of the bladder, blood, bone, brain, breast, cervix, colon/rectum, endometrium, head and neck, kidney, liver, lung, lymph nodes, muscle tissue, ovary, pancreas, prostate, skin, spleen, stomach, testicle, thyroid, or uterus.

In some embodiments, the cancer is invasive and/or metastatic cancer (e.g., stage II cancer, stage III cancer or stage IV cancer). In some embodiments, the cancer is an early stage cancer (e.g., stage 0 cancer, stage I cancer), and/or is not invasive and/or metastatic cancer.

In some embodiments, the disease or disorder is an infectious disease. Infectious diseases that can be treated or prevented by the methods of the present invention are caused by infectious agents including, but not limited to, viruses, bacteria, fungi, protozoa, helminths, and parasites. The invention is not limited to treating or preventing infectious diseases caused by intracellular or extracellular pathogens. The infectious disease may be derived from: bacteria, such as Mycobacterium tuberculosis, Chlamydia, Francisella tularensis; DNA viruses, such as Herpesviridae (herpes simplex virus-1, Kaposi's sarcoma-associated virus and Epstein-Barr virus), Papillomaviridae (human papilloma virus), Adenovirus and Hepadnaviridae (Hepatitis B virus), or RNA viruses, such as Retroviridae (human immunodeficiency virus) Flaviviridae (Dengue virus, Hepatitis C virus), Orthomyxoviridae (influenza), and Coronaviridae (human coronavirus and SARS coronavirus).

The compounds and compositions disclosed herein may be administered to a subject by a variety of methods. In any of the uses or methods described herein, administration may be by various routes known to those skilled in the art, including without limitation oral, inhalation, intravenous, intramuscular, topical, subcutaneous, systemic, and/or intraperitoneal administration to a subject in need thereof.

The amount of the compounds of the present disclosure required for use in the disclosed methods will vary not only with the particular compound selected but also with the route of administration, the nature and/or symptoms of the disease and the age and condition of the patient and will be ultimately at the discretion of the attendant physician or clinician. The determination of effective dosage levels, that is the dosage levels necessary to achieve the desired result, can be accomplished by one skilled in the art using routine methods, for example, human clinical trials, in vivo studies, and in vitro studies. For example, useful dosages can be determined by comparing their in vitro activity, and in vivo activity in animal models.

Dosage amount and interval may be adjusted individually to provide plasma levels of the active moiety which are sufficient to maintain the modulating effects, or minimal effective concentration (MEC). The MEC will vary for each compound but can be estimated from in vivo and/or in vitro data. Dosages necessary to achieve the MEC will depend on individual characteristics and route of administration. However, FIPLC assays or bioassays can be used to determine plasma concentrations. Dosage intervals can also be determined using MEC value. Compositions should be administered using a regimen, which maintains plasma levels above the MEC for 10-90% of the time, preferably between 30-90% and most preferably between 50-90%. In cases of local administration or selective uptake, the effective local concentration of the drug may not be related to plasma concentration.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the symptoms to be treated and the route of administration. Further, the dose, and perhaps dose frequency, will also vary according to the age, body weight, and response of the individual patient. A program comparable to that discussed above may be used in veterinary medicine.

The compounds and compositions disclosed herein can be evaluated for efficacy and toxicity using known methods. For example, the toxicology of a particular compound or a subset of the compounds sharing certain chemical moieties, or a composition thereof, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, dogs, or monkeys, may be determined using known methods. Efficacy may be established using several recognized methods, such as in vitro methods, animal models, or human clinical trials. When selecting a model to determine efficacy, the skilled artisan can be guided by the state of the art to choose an appropriate model, dose, route of administration and/or regime.

A wide range of second therapies may be used in conjunction with the compounds of the present disclosure. The second therapy may be administration of an additional therapeutic agent or may be a second therapy not connected to administration of another agent. Such second therapies include, but are not limited to, surgery, immunotherapy, radiotherapy.

The second therapy may be administered at the same time as the initial therapy, either in the same composition or in a separate composition administered at substantially the same time as the first composition. In some embodiments, the second therapy may precede or follow the treatment of the first therapy by time intervals ranging from hours to months.

In some embodiments, a therapeutically effective amount of a compound disclosed herein, or compositions thereof, is administered alone or in combination with a therapeutically effective amount of at least one additional therapeutic agent. In some embodiments, effective combination therapy is achieved with a single composition or pharmacological formulation that includes both agents, or with two distinct compositions or formulations, administered at the same time or separated by a time interval, wherein one composition includes a compound of this invention, and the other includes the at least one additional therapeutic agent.

In some embodiments, the at least one additional therapeutic agent comprises an immune modulator, a chemotherapeutic agent, a nucleic acid (e.g., mRNA, aptamers, antisense oligonucleotides, ribozyme nucleic acids, interfering RNAs, antigene nucleic acids), a decongestant, a steroid, an analgesic, an antimicrobial agent, an immunotherapy, or a combination thereof.

Exemplary immune modulators include: indoleamine 2,3-dioxygenase (IDO) inhibitors and analogs thereof, such as, epacadostat, BMS-986205, indoximod, PF-06840003, and analogs thereof; signal transducer and activator of transcription 3 (Stat3) inhibitors and analogs thereof, such as, SM-36 and its analogs; toll-like receptor (TLR) agonists and analogs thereof, such as, imiquimod, resiquimod, selgantolimod, gardiquimod, SM-360320, TMX-101, TMX-202, TMX-302, TMX-306, GSK2245035, CL097, 852A, AZD-8848, DSP-3025, GS-9620, RO7020531, RO6871765, ANA773, DSP-0509, NJH395, BNT411, TQ-A3334, JNJ-4964, LHC165, CV8102, VTX-1463, VTX-2337, IMO-8400, IMO-3100, IRS-954, and analogs thereof; and statins or other lipid-lowering medications and analogs thereof, such as, atorvastatin, pravastatin, fluvastatin, simvastatin, lovastatin, mevastatin, pitavastatin, rosuvastatin, and analogs thereof.

In some embodiments, the at least one additional therapeutic agent comprises at least one chemotherapeutic agent. As used herein, the term “chemotherapeutic” or “anti-cancer drug” includes any small molecule or other drug used in cancer treatment or prevention. Chemotherapeutics include, but are not limited to, cyclophosphamide, methotrexate, 5-fluorouracil, doxorubicin, docetaxel, daunorubicin, bleomycin, vinblastine, dacarbazine, cisplatin, paclitaxel, raloxifene hydrochloride, tamoxifen citrate, abemacicilib, afinitor (Everolimus), alpelisib, anastrozole, pamidronate, anastrozole, exemestane, capecitabine, epirubicin hydrochloride, eribulin mesylate, toremifene, fulvestrant, letrozole, gemcitabine, goserelin, ixabepilone, emtansine, lapatinib, olaparib, megestrol, neratinib, palbociclib, ribociclib, talazoparib, thiotepa, toremifene, methotrexate, and tucatinib. In select embodiments, the chemotherapeutic agent comprises paclitaxel.

In some embodiments of the methods disclosed herein, the compound or composition can be co-administered with an antimicrobial (e.g., antiviral or antibacterial) agent. In some embodiments, the additional antimicrobial agent is an antiviral agent, including but not limited to, abacavir, acyclovir, adefovir, amantadine, amprenavir, atazanavir, haloxavir marboxil, bictegravir, boceprevir, bulevirtide, cidofovir, cobicistat, daclatasvir, darunavir, delavirdine, didanosine, docosanol, dolutegravir, doravirine, edoxudine, efavirenz, elvitegravir, emtricitabine, enfuvirtide, entecavir, etravirine, famciclovir, fomivirsen, fosamprenavir, foscarnet, ganciclovir, ibacitabine, ibalizumab, idoxuridine, imiquimod, imunovir, indinavir, lamivudine, letermovir, lopinavir, loviride, maraviroc, methisazone, moroxydine, nelfinavir, nevirapine, nexavir, nitazoxanide, oseltamivir, penciclovir, peramivir, penciclovir, peramivir, pleconaril, podophyllotoxin, raltegravir, remdesivir, ribavirin, rilpivirine, rilpivirine, rimantadine, rintatolimod, ritonavir, saquinavir, simeprevir, sofosbuvir, stavudine, taribavirin, telaprevir, telbivudine, tenofovir (e.g., tenofovir alafenamide or tenofovir disoproxil), tipranavir, trifluridine, tromantadine, umifenovir, valaciclovir, valganciclovir, vicriviroc, vidarabine, zalcitabine, zanamivir, and zidovudine, and any combination thereof.

In some embodiments, the additional antimicrobial agent is an antibacterial agent. Exemplary antibacterial agents include sulfonamides, amphenicols, spectinomycin, trimethoprim, glycylcyclines, macrolides (e.g., erythromycin, clarithromycin, azithromycin, roxithromycin), oxazolidinones (e.g., linezolid), tetracyclines (e.g., doxycycline, tetracycline, minocycline), β-lactams (e.g., penicillin, methicillin, cloxacillin), carbapenems (e.g., imipenem, meropenem, aztreonam), aminoglycosides (e.g., gentamicin, tobramycin, amikacin), quinolones and fluoroquinolones (e.g., levofloxacin, ciprofloxacin, moxifloxacin), glycopeptides (e.g., vancomycin), polymyxins (e.g., polymyxin, colistin).

In some embodiments, the second therapy includes immunotherapy.

Immunotherapies include chimeric antigen receptor (CAR) T-cell or T-cell transfer therapies, cytokine therapy, immunomodulators, cancer vaccines, or administration of antibodies (e.g., monoclonal antibodies).

In some embodiments, the immunotherapy comprises administration of antibodies. The antibodies may target antigens either specifically expressed by tumor cells or antigens shared with normal cells. In some embodiments, the immunotherapy may comprise an antibody targeting, for example, CD20, CD33, CD52, CD30, HER (also referred to as erbB or EGFR), VEGF, CTLA-4 (also referred to as CD152), epithelial cell adhesion molecule (EpCAM, also referred to as CD326), and PD-1/PD-L¹. Suitable antibodies include, but are not limited to, rituximab, blinatumomab, trastuzumab, gemtuzumab, alemtuzumab, ibritumomab, tositumomab, bevacizumab, cetuximab, panitumumab, ofatumumab, ipilimumab, brentuximab, pertuzumab, and the like). In some embodiments, the additional therapeutic agent may comprise anti-PD-1/PD-L1 antibodies, including, but not limited to, pembrolizumab, nivolumab, cemiplimab, atezolizumab, avelumab, durvalumab, and ipilimumab. The antibodies may also be linked to a chemotherapeutic agent. Thus, in some embodiments, the antibody is an antibody-drug conjugate.

The immunotherapy (e.g., administration of antibodies) may be administered to a subject by a variety of methods. In any of the uses or methods described herein, administration may be by various routes known to those skilled in the art, including without limitation oral, inhalation, intravenous, intramuscular, topical, subcutaneous, systemic, and/or intraperitoneal administration to a subject in need thereof. The immunotherapy may be administered by parenteral administration (including, but not limited to, subcutaneous, intramuscular, intravenous, intraperitoneal, intracardiac and intraarticular injections). In some embodiments, the immunotherapy may be administered in the same or different manner than the disclosed compounds or compositions.

### 5. KITS

In another aspect, the disclosure provides kits comprising at least one disclosed compound or a pharmaceutically acceptable salt thereof, or a composition comprising the compound or a pharmaceutically acceptable salt thereof, and instructions for using the compound or composition.

The kits can also comprise other agents and/or products co-packaged, co-formulated, and/or co-delivered with other components. For example, a drug manufacturer, a drug reseller, a physician, a compounding shop, or a pharmacist can provide a kit comprising a disclosed compound and/or product and another agent for delivery to a patient.

The kits can also comprise instructions for using the components of the kit. The instructions are relevant materials or methodologies pertaining to the kit. The materials may include any combination of the following: background information, list of components, brief or detailed protocols for using the compositions, trouble-shooting, references, technical support, and any other related documents. Instructions can be supplied with the kit or as a separate member component, either as a paper form or an electronic form which may be supplied on computer readable memory device or downloaded from an internet website, or as recorded presentation.

It is understood that the disclosed kits can be employed in connection with the disclosed methods. The kit may further contain containers or devices for use with the methods or compositions disclosed herein. The kits optionally may provide additional components such as buffers and disposable single-use equipment (e.g., pipettes, cell culture plates or flasks).

The kits provided herein are in suitable packaging. Suitable packaging includes, but is not limited to, vials, bottles, jars, flexible packaging, and the like. Individual member components of the kits may be physically packaged together or separately.

### 6. EXAMPLES

Abbreviations used in the schemes and examples that follow are: Boc is tert-butyloxycarbonyl; Boc₂O is di-tert-butyl dicarbonate; DCM is dichloromethane; DMAP is 4-dimethylaminopyridine; EDC is 1-ethyl-3-(3-dimethylaminopropyl) carbodiimide; eq is equivalent; EtOAc is ethyl acetate; MeOH is methanol; RT or r.t. is room temperature; and THF is tetrahydrofuran.

All air and moisture sensitive manipulations were performed under either argon or in vacuo using standard Schlenk techniques. Anhydrous solvents (Et2O, THE, Dioxane, DMSO, DMF, DCM and Toluene) were purchased from Fisher Scientific. All chemicals were purchased from Fisher Scientific, Sigma Aldrich, TCI WUXI Apptec and DC Chemicals Europe and were used without further purification unless mentioned otherwise.

Analytical thin layer chromatography (TLC) was performed with Merck SIL G/UV254 plates. Compounds were visualized by exposure to UV light or by dipping the plates in solutions of ninhydrin or potassium permanganate followed by heating or by staining with Iodine vapor in a wide jar chamber. Column chromatography was performed in air with silicagel 60 (Fluka). Column chromatography was performed with Merck Kieselgel 60 (200-500 mm). The solvent systems were given (s/s v:v).

NMR spectra ¹H (300 MHz), ¹³C (75 MHz) were respectively recorded on an ARX 300 or an Avance II 500 Bruker spectrometer. Chemical shifts (δ, ppm) are given with reference to residual 1H or 13C of deuterated solvents in the solvent indicated (CDCl 7.26, 77.00; (CD₃)₂CO 2.05, 29.84 and 206.26, (CD₃)₂SO 2.50, 39.52)). 1H- and 13C-NMR chemical shifts (δ) are quoted in parts per million (ppm) relative to the TMS scale. Coupling constants J are quoted in Hz. The following abbreviations are used for the proton spectra multiplicities: s: singlet, d: doublet, t: triplet, q: quartet, qt: quintuplet, m: multiplet, br.: broad, dd: double doublet, dt: double triplet. Coupling constants (J) are reported in Hertz (Hz). Several signals could not be attributed will be represented by ArH (aromatic hydrogen).

Mass spectra (MS) were recorded with a LCQ-advantage (ThermoFinnigan) mass spectrometer with positive (ESI+) or negative (ESI−) electrospray ionization (ionization tension 4.5 kV, injection temperature 240° C.).

### Example 1

### Compound Synthesis

### General Procedure 1: Esterification

Under a nitrogen atmosphere, a solution of the carboxylic acid derivative (1 eq.), EDC hydrochloride (1 eq.), and DMAP (0.5 eq.) in dry THF (0.1 M) was stirred at 0° C. for 0.5 h. Then after, a solution of the alcohol (1 eq.) in dry THF (0.1 M) was added. The solution was stirred at 0° C. to r.t. for 16 h. Progress of the reaction was monitored by TLC using CH₂—Cl₂-MeOH 10:1 mixture as eluent and 1H NMR using ARX 300 Brucker spectrometer. Upon completion of the reaction, the mixture was extracted with dichloromethane (3×). The combined organic layer and washed with 0.2 M HCl aqueous, saturated aqueous solution of NaHCO₃ and brine, respectively. Then, the solid residue was purified by manual column chromatography using CH₂Cl₂-EtOAc or CH₂Ch-MeOH step gradient solvent system as an eluent to afford the titled product.

Synthesis of the dual functional compounds used the above described general esterification procedure as described in the steps below.

### 3-((1-methyl-L-tryptophyl)oxy) propyl 4-(5,6-dimethoxybenzo[b]thiophen-2-yl)-4-oxobutanoate

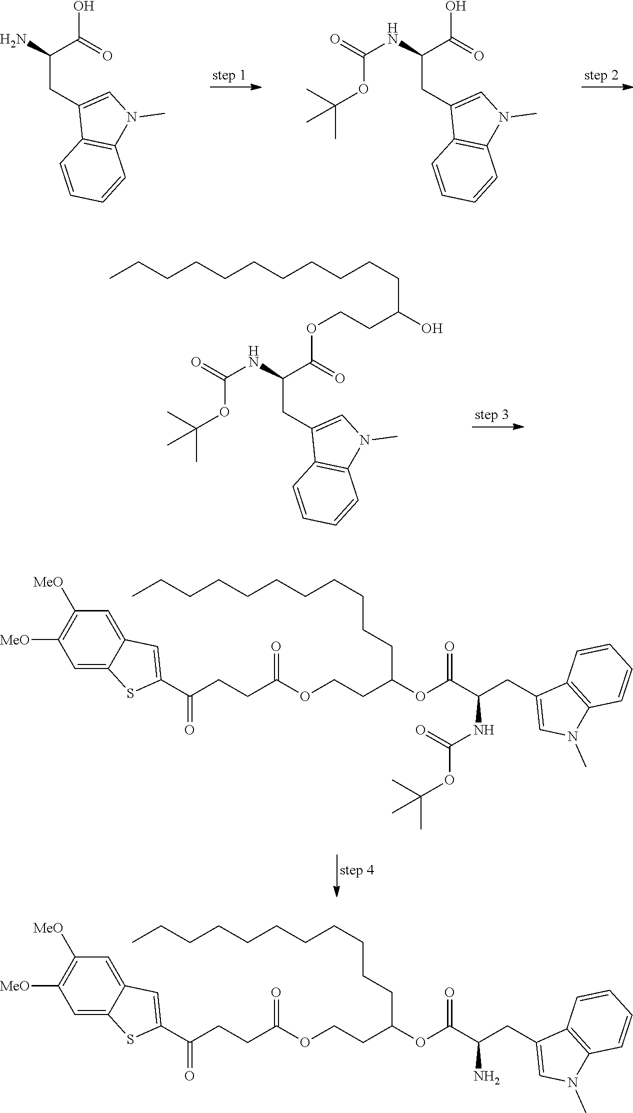 [SMILES: Cn1cc(C[C@@H](N)C(=O)O)c2ccccc21 ; Cn1cc(C[C@@H](NC(=O)OC(C)(C)C)C(=O)O)c2ccccc21 ; CCCCCCCCCCCC(O)CCOC(=O)[C@@H](Cc1cn(C)c2ccccc12)NC(=O)OC(C)(C)C ; CCCCCCCCCCCC(CCOC(=O)CCC(=O)c1cc2cc(OC)c(OC)cc2s1)OC(=O)[C@@H](Cc1cn(C)c2ccccc12)NC(=O)OC(C)(C)C ; CCCCCCCCCCCC(CCOC(=O)CCC(=O)c1cc2cc(OC)c(OC)cc2s1)OC(=O)[C@H](N)Cc1cn(C)c2ccccc12]

Step 1: N^{α}-(tert-butoxycarbonyl)-1-methyl-D-tryptophan. An anhydrous DCM solution of 1-methyl-D-tryptophan (1 eq) was treated by trimethylamine (2.5 eq) and Boc₂ (O) (1.05 eq) at 0° C. to RT over 16 h. The mixture was washed with 1 N HCl aqueous solution and brine to afford the desired product.

Step 2: 3-hydroxypropyl N^{α} (tert-butoxycarbonyl)-1-methyl-D-tryptophanate. The titled compound was synthesized according to the General Procedure 1 from N^{α}-(tert-butoxycarbonyl)-1-methyl-D-tryptophan (1 eq), 1,2 tetradecanediol (1.2 eq), EDC. hydrochloride (1 eq.), DMAP (0.5 eq.) in dry THF (0.1 M) during 4 h at room temperature.

Step 3: 3-((N^{α}-(tert-butoxycarbonyl)-1-methyl-D-tryptophyl)oxy) propyl 4-(5,6-dimethoxybenzo[b]thiophen-2-yl)-4-oxobutanoate. The titled compound was synthesized according to the General Procedure 1 from 3-hydroxypropyl N^{α} (tert-butoxycarbonyl)-1-methyl-D-tryptophanate (1 eq), 4-(5,6-dimethoxybenzo[b]thiophen-2-yl)-4-oxobutanoic acid (1 eq), EDC. hydrochloride (1 eq.), DMAP (0.5 eq.) in dry THF (0.1 M) during 4 h at room temperature.

Step 4: 3-((1-methyl-D-tryptophyl)oxy) propyl 4-(5,6-dimethoxybenzo[b]thiophen-2-yl)-4-oxobutanoate The titled compound was synthesized from 3-((N^{α}-(tert-butoxycarbonyl)-1-methyl-D-tryptophyl)oxy) propyl 4-(5,6-dimethoxybenzo[b]thiophen-2-yl)-4-oxobutanoate (1 eq) stirred in HCl solution of dioxane (HCl 4 M in dioxane) from 0° C. to RT during 5 h.

### (R)-2-(4-(3-(1-(2-aminopyrazolo[1,5-a]pyrimidine-3-carboxamido)ethyl)-1-oxo-2-phenyl-1,2-dihydroisoquinolin-8-yl)-1H-1,2,3-triazol-1-yl)ethyl 4-(5,6-dimethoxybenzo[b]thiophen-2-yl)-4-oxobutanoate (DMA01-166)

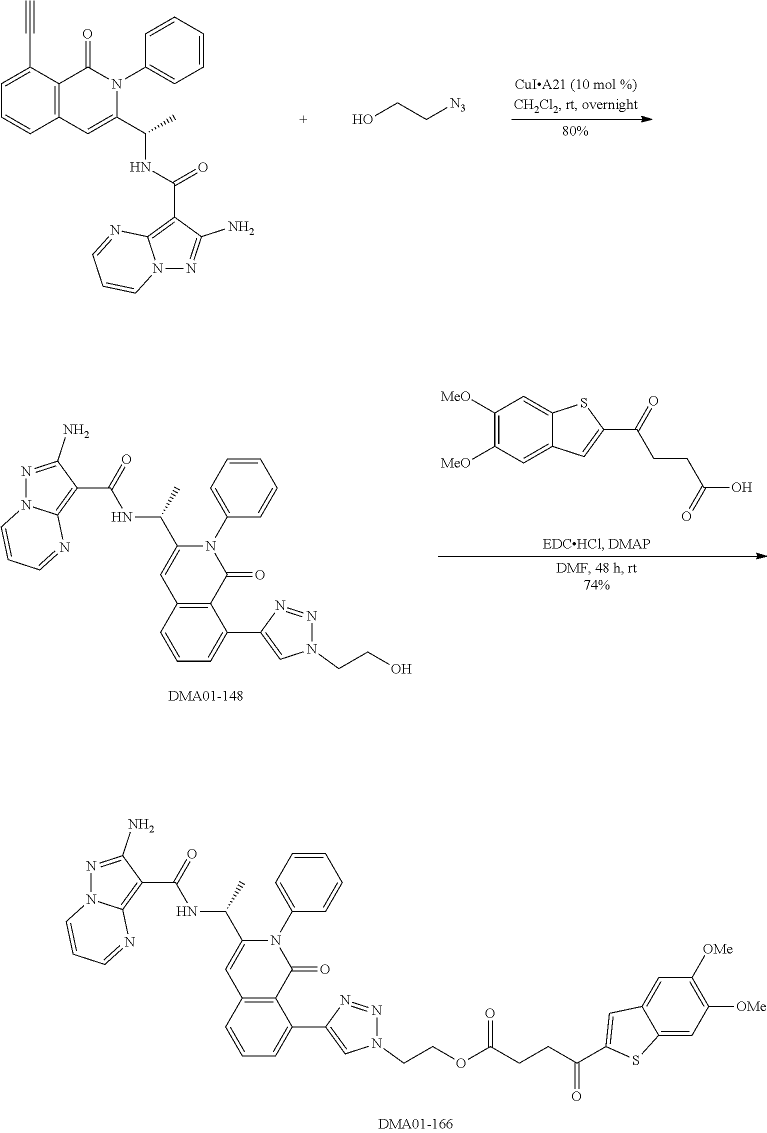 [SMILES: C#Cc1cccc2cc([C@H](C)NC(=O)c3c(N)nn4cccnc34)n(-c3ccccc3)c(=O)c12 ; [N-]=[N+]=NCCO ; C[C@@H](NC(=O)c1c(N)nn2cccnc12)c1cc2cccc(-c3cn(CCO)nn3)c2c(=O)n1-c1ccccc1 ; COc1cc2cc(C(=O)CCC(=O)O)sc2cc1OC ; COc1cc2cc(C(=O)CCC(=O)OCCn3cc(-c4cccc5cc([C@@H](C)NC(=O)c6c(N)nn7cccnc67)n(-c6ccccc6)c(=O)c45)nn3)sc2cc1OC]

Synthesis of DMA01-148: To a solution of(S)-2-amino-N-(1-(8-ethynyl-1-oxo-2-phenyl-1,2-dihydroisoquinolin-3-yl)ethyl) pyrazolo[1,5-a]pyrimidine-3-carboxamide (100 mg, 0.22 mmol) and 2-azidoethanol (38.83 mg, 0.44 mmol) in CH₂Cl₂ (3 mL) was added CuI.A-21 catalyst (16.5 mg, 10 mol %). The reaction mixture was sonicated for 1 h and stirred at room temperature overnight. Upon completion of the reaction, the catalyst was filtered off using CH₂Cl₂ and the solvent was evaporated under vacuum. The crude product was then purified by column chromatography to provide compound DMA01-148 (95 mg, 80% yield). H NMR (500 MHZ, CDCl₃) δ 8.44 (dd, J=23.4, 5.4 Hz, 2H), 8.03-7.83 (m, 2H), 7.74 (d, J=7.4 Hz, 1H), 7.62 (t, J=7.7 Hz, 1H), 7.54-7.35 (m, 5H), 6.80 (dd, J=6.5, 4.4 Hz, 1H), 6.71 (s, 1H), 4.80 (p, J=6.8 Hz, 1H), 4.46-4.25 (m, 2H), 3.89 (t, J=4.9 Hz, 2H), 1.41 (d, J=6.8 Hz, 3H).

Synthesis of DMA01-166 (esterification): A solution of 4-(5,6-dimethoxybenzo[b]thiophen-2-yl)-4-oxobutanoic acid (48.27 mg, 0.16 mmol) and EDC hydrochloride (28.65 mg, 0.15 mmol) in DMF (3 mL) was stirred at 0° C. for 0.5 h. DMA01-148 (80 mg, 0.15 mmol) and DMAP (18.25 mg, 0.15 mmol) were added and the reaction mixture was stirred at 0° C. for 1 h before letting to stir at room temperature for 48 h. Upon completion of the reaction, the reaction mixture was diluted in CH₂Cl and washed with saturated aqueous solution of NaHCO₃, water and brine respectively. The organic layer was dried over Na₂SO₄, filtered and concentrated under vacuum. Purification by column chromatography provided compound DMA01-166 (90 mg, 74% yield). ¹H NMR (500 MHZ, CDCl₃) δ 8.48-8.39 (m, 2H), 8.09 (s, 1H), 7.95 (d, J=7.0 Hz, 1H), 7.87 (dd, J=7.5, 1.4 Hz, 1H), 7.82 (8, 1H), 7.61 (t, J=7.7 Hz, 1H), 7.53-7.36 (m, 5H), 7.22 (s, 1H), 7.18 (s, 1H), 6.81 (dd, J=6.7, 4.4 Hz, 1H), 6.69 (s, 1H), 4.80 (p, J=6.8 Hz, 1H), 4.59 (dd, J=5.8, 4.3 Hz, 2H), 4.49 (t, J=5.1 Hz, 2H), 3.97 (s, 3H), 3.92 (s, 3H), 3.26-3.03 (m, 2H), 2.64 (td, J=6.6, 1.8 Hz, 2H), 1.40 (d, J=6.8 Hz, 3H).

### 2-(4-(3-((R)-1-(2-aminopyrazolo[1,5-a]pyrimidine-3-carboxamido)ethyl)-1-oxo-2-phenyl-1,2-dihydroisoquinolin-8-yl)-1H-1,2,3-triazol-1-yl)ethyl (9Z,12E)-octadeca-9,12-dienoate (DMA01-170)

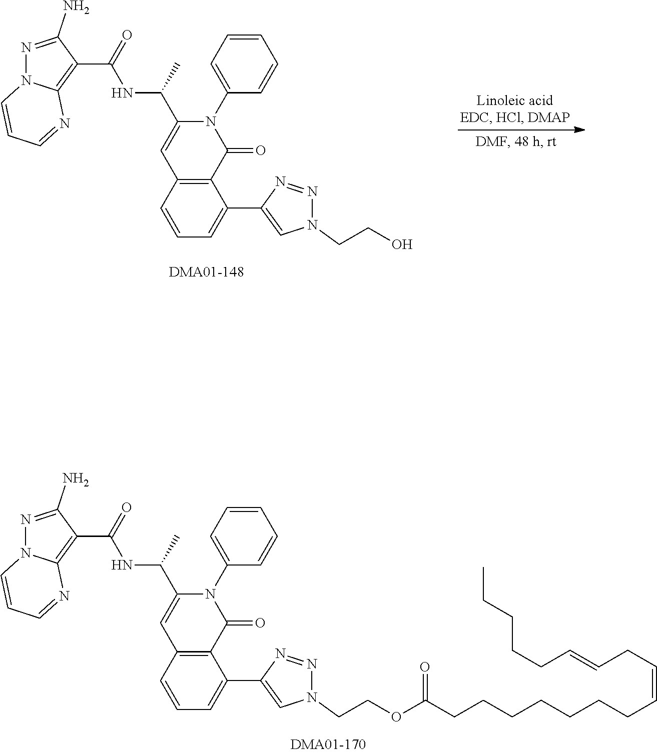 [SMILES: C[C@@H](NC(=O)c1c(N)nn2cccnc12)c1cc2cccc(-c3cn(CCO)nn3)c2c(=O)n1-c1ccccc1 ; CCCCC/C=C/C/C=C\CCCCCCCC(=O)OCCn1cc(-c2cccc3cc([C@@H](C)NC(=O)c4c(N)nn5cccnc45)n(-c4ccccc4)c(=O)c23)nn1]

Synthesis of DMA01-170: The compound DMA01-170 was obtained from DMA01-148 and linoleic acid following the esterification method described above.

### 2-(4-(3-((R)-1-(2-aminopyrazolo[1,5-a]pyrimidine-3-carboxamido)ethyl)-1-oxo-2-phenyl-1,2-dihydroisoquinolin-8-yl)-1H-1,2,3-triazol-1-yl)ethyl 1-methyl-D-tryptophanate (DMA01-173)

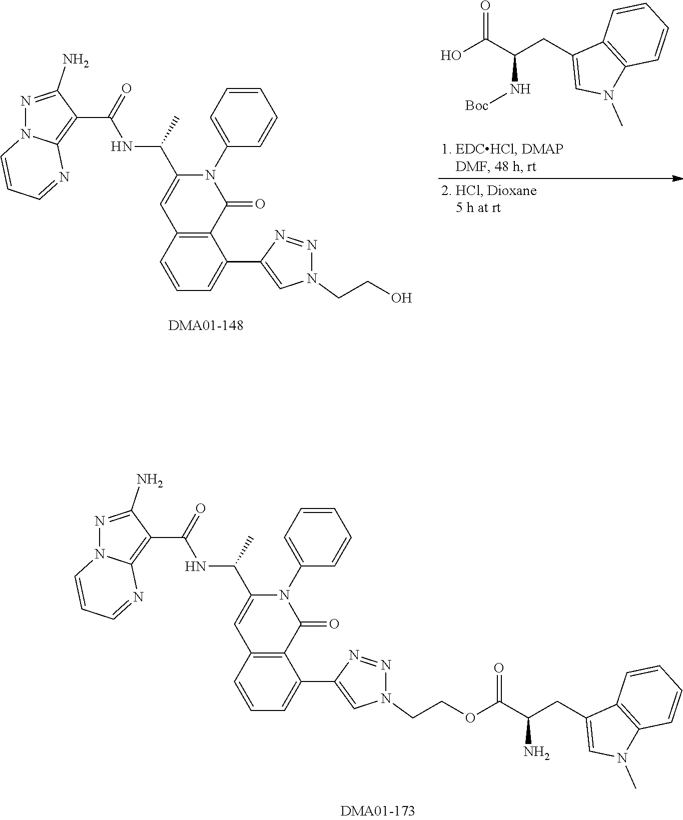 [SMILES: C[C@@H](NC(=O)c1c(N)nn2cccnc12)c1cc2cccc(-c3cn(CCO)nn3)c2c(=O)n1-c1ccccc1 ; Cn1cc(C[C@@H](NC(=O)OC(C)(C)C)C(=O)O)c2ccccc21 ; C[C@@H](NC(=O)c1c(N)nn2cccnc12)c1cc2cccc(-c3cn(CCOC(=O)[C@H](N)Cc4cn(C)c5ccccc45)nn3)c2c(=O)n1-c1ccccc1]

Synthesis of DMA01-173: The compound DMA01-173 was obtained from DMA01-148 and N^{α}-(tert-butoxycarbonyl)-1-methyl-D-tryptophan following the esterification method described above and deprotection of the Boc with HCl in dioxane.

### (S)-2-(4-(3-(1-(2-aminopyrazolo[1,5-a]pyrimidine-3-carboxamido)ethyl)-1-oxo-2-phenyl-1,2-dihydroisoquinolin-8-yl)-1H-1,2,3-triazol-1-yl) acetic acid (DMA01-143)

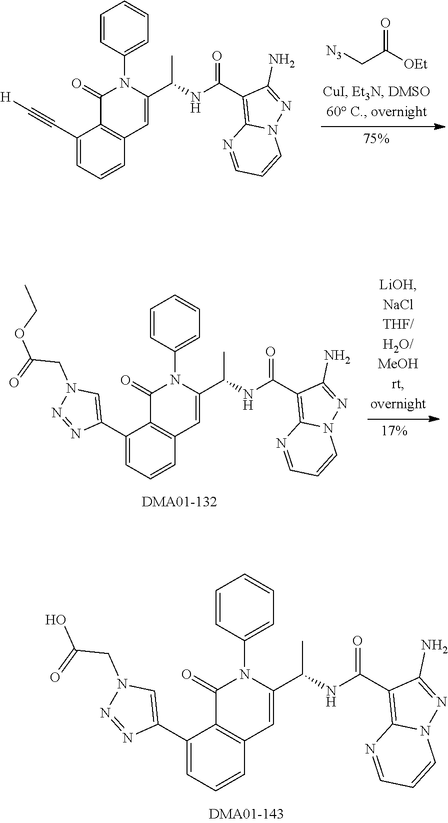 [SMILES: C#Cc1cccc2cc([C@H](C)NC(=O)c3c(N)nn4cccnc34)n(-c3ccccc3)c(=O)c12 ; CCOC(=O)CN=[N+]=[N-] ; CCOC(=O)Cn1cc(-c2cccc3cc([C@H](C)NC(=O)c4c(N)nn5cccnc45)n(-c4ccccc4)c(=O)c23)nn1 ; C[C@H](NC(=O)c1c(N)nn2cccnc12)c1cc2cccc(-c3cn(CC(=O)O)nn3)c2c(=O)n1-c1ccccc1]

Synthesis of DMA01-132: (S)-2-amino-N-(1-(8-ethynyl-1-oxo-2-phenyl-1,2-dihydroisoquinolin-3-yl)ethyl) pyrazolo[1,5-a]pyrimidine-3-carboxamide (155 mg, 0.35 mmol), Ethyl azidoacetate (25% in toluene, 180 μL, 0.35 mmol, 1 eq) and CuI (8 mg, 10 mol %) in DMSO (0.9 mL, 0.4 M) was triturated in sonic bath during 0.5 h then heated at 60° C. overnight, after which column purification (SiO₂, CH₂Cl₂-MeOH 97:3) afforded the desired product as an off-white solid (150 mg, 75% yield). ¹H NMR (300 MHZ, MeOD) δ 8.66 (dd, J=6.8, 1.6 Hz, 1H), 8.49 (dd, J=4.5, 1.6 Hz, 1H), 8.02 (8, 1H), 7.79-7.27 (m, 8H), 7.00-6.84 (m, 2H), 5.25 (s, 2H), 4.75 (t, J=6.8 Hz, 1H), 4.21 (q, J=7.1 Hz, 2H), 1.45 (d, J=6.8 Hz, 3H), 1.25 (t, J=7.1 Hz, 3H).

Synthesis of DMA01-143: DMA01-132 (31 mg, 0.05 mmol, 1 eq) in THF (1 mL) was treated with an aqueous solution of LiOH (2 mg 0.08 mmol, 1.5 eq) and NaCl (5 mg, 0.081 mmol, 1.5 eq) in 0.3 mL of H₂O. Methanol (0.3 mL) was added to the mixture and the reaction was stirred at room temperature overnight, after which pTLC purification (SiO₂, CH₂Cl₂-MeOH 9:1, duration: 18 h) afforded the desired product as an off-white solid (5 mg, 17% yield). ¹H NMR (300 MHz, MeOD) δ 8.70 (dd, J=6.7, 1.7 Hz, 1H), 8.51 (dd, J=4.5, 1.6 Hz, 1H), 8.21 (d, J=7.0 Hz, 1H), 7.95 (s, 1H), 7.78-7.69 (m, 2H), 7.69-7.61 (m, 1H), 7.58-7.31 (m, 5H), 7.02-6.91 (m, 2H), 5.49 (s, 2H), 4.79 (t, J=6.9 Hz, 1H), 1.47 (d, J=6.7 Hz, 3H).

### 1-(4-(3-((R)-1-(2-aminopyrazolo[1,5-a]pyrimidine-3-carboxamido)ethyl)-1-oxo-2-phenyl-1,2-dihydroisoquinolin-8-yl)-1H-1,2,3-triazol-1-yl) propan-2-yl 4-(5,6-dimethoxybenzo[b]thiophen-2-yl)-4-oxobutanoate (SHI-233)

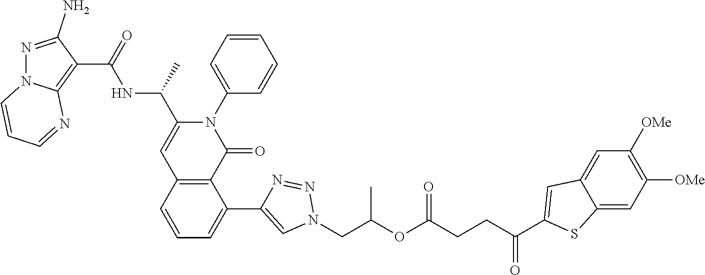 [SMILES: COc1cc2cc(C(=O)CCC(=O)OC(C)Cn3cc(-c4cccc5cc([C@@H](C)NC(=O)c6c(N)nn7cccnc67)n(-c6ccccc6)c(=O)c45)nn3)sc2cc1OC]

SH-233 was synthesized by methods similar to those shown for DMA01-166 above, using 2-azidopropan-1-ol in place of 2-azidoethanol. ¹H NMR (500 MHz, cdcl₃) δ 8.44 (dd, J=14.6, 5.7 Hz, 2H), 8.15-7.57 (m, 5H), 7.54-7.32 (m, 7H), 7.25-7.10 (m, 3H), 6.81 (t, J=5.7 Hz, 1H), 6.67 (t, J=6.1 Hz, 1H), 5.57 (s, 2H), 4.83-4.74 (m, 1H), 4.40 (dd, J=14.6, 6.6 Hz, 1H), 4.00-3.95 (m, 3H), 3.95-3.90 (m, 3H), 3.64 (s, 1H), 3.36 (d, J=12.3 Hz, 1H), 3.12-2.99 (m, 1H), 2.60 (q, J=6.8 Hz, 1H), 1.39 (dd, J=9.6, 7.0 Hz, 6H).

### Example 2

### STING Activation by Dual Functioning NCEs

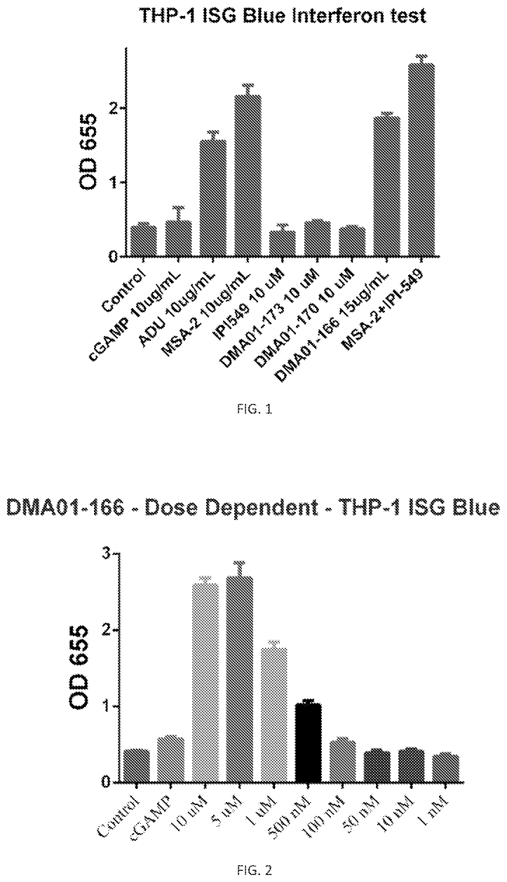

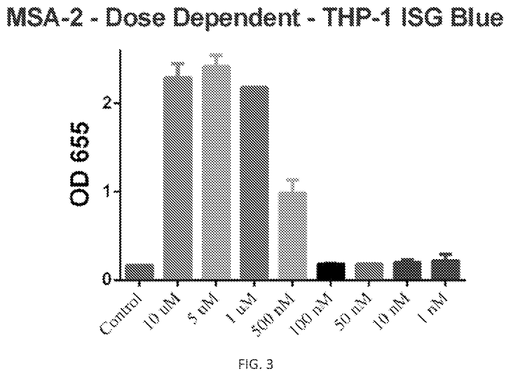

THP-1 ISG Blue cells analysis is a standard assay to test STING activation in vitro. DMA01-166 showed enhanced interferon signal compared to control and cGAMP group. DMA01-166 showed similar STING activation compared to MSA-2 and ADU, which are both potent STING agonists (FIG. 1). In contrast, DMA01-173 did not stimulate STING activation. Different concentrations of DMA01-166 (FIG. 2) and MSA-2 (FIG. 3) were incubated with THP-1 ISG Blue cells for 24 hours. Both DMA01-166 and MSA-2 showed similar dose independent STING activation effect.

### Example 3

### Bone Marrow Dendritic Cell (BMDC) Activation by Dual Functioning NCEs

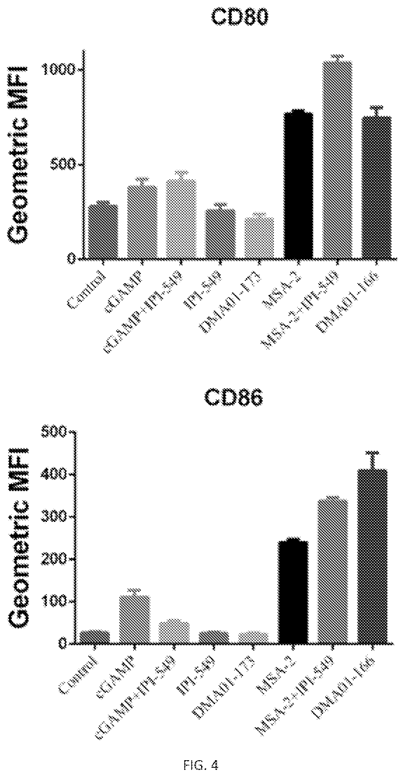

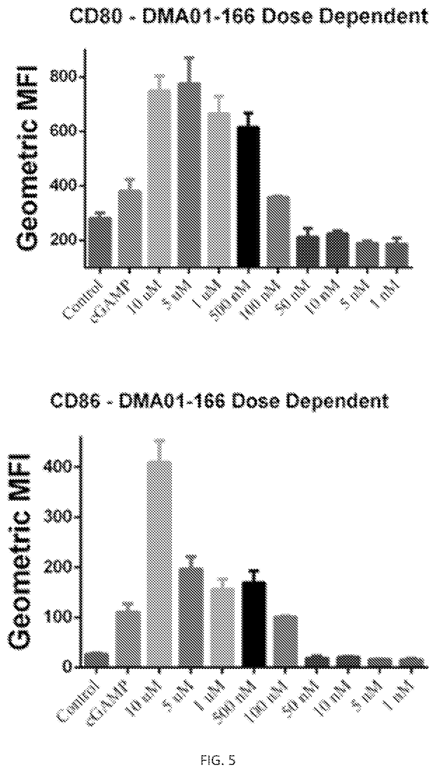

STING activation induces BMDC activation. To test the ability of DMA01-166 in activating BMDC, BMDCs activation was measured based on the upregulation of CD80 and CD86 by flow cytometry. DMA01-166 showed similar STING activation compared to MSA-2 (FIG. 4). In contrast, DMA01-173 did not stimulate DC activation. Different concentrations of DMA01-166 were incubated with BMDC cells for 24 hours. DMA01-166 showed a dose independent BMDC activation effect (FIG. 5).

### Example 4

### mIFNβ Secretion Induced Dual Functioning NCEs

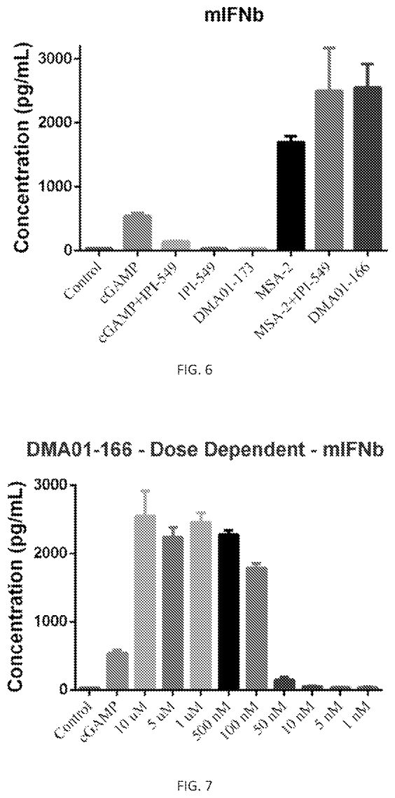

STING activation in BMDC results in mIFNβ secretion. DMA01-166 showed higher mIFNβ secretion compared to MSA-2 (FIG. 6). In contrast, DMA01-173 cannot enhance mIFNβ secretion. Different concentrations of DMA01-166 (FIG. 7) were incubated with BMDC cells for 24 hours. DMA01-166 showed a dose independent BMDC activation effect.

### Example 5

### Macrophage Polarization by Dual Functioning NCEs

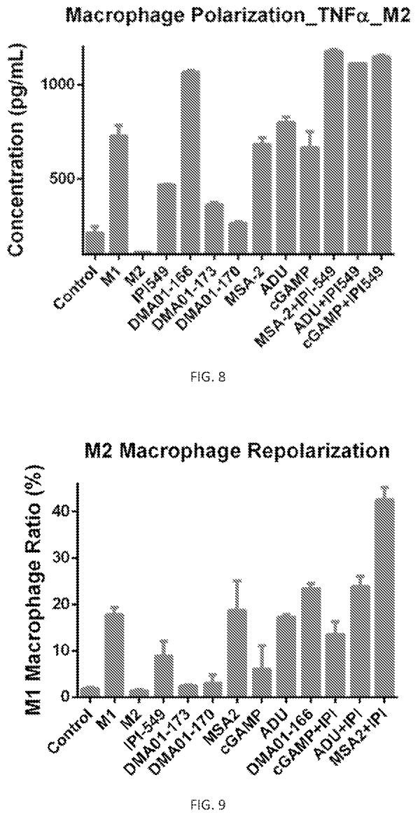

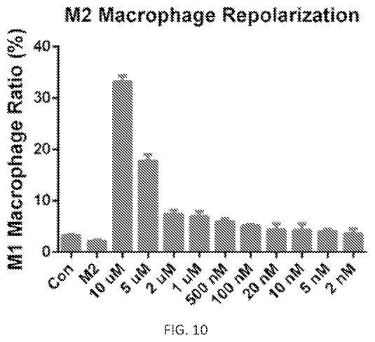

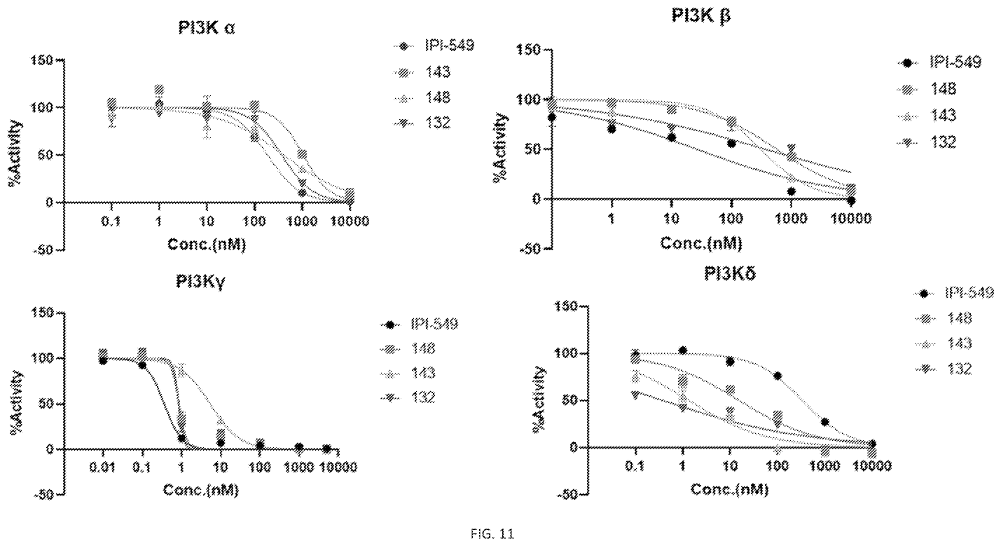

STING activation and PI3K inhibition both induced macrophage polarization from M2 to M1 phenotype for higher TNFα secreting compared to IPI-549, MSA-2, ADU and cGAMP, four compounds which are well studied for their effectiveness on macrophage polarization (FIG. 8). DMA01-173 and DMA01-170 also showed macrophage polarization effects compared to M2 control group (FIG. 8). Macrophage polarization effects of the dual functioning NCEs were further tested by first inducing RAW264.7 cells to M2 macrophage, and then treating with different drugs to test the M1 macrophage percentage among all the macrophages. DMA01-166 induced higher percentage of M1 macrophage compared to IPI-549, cGAMP, ADU and MSA-2. DMA01-173 and DMA01-170 also showed mild effects on increasing M1 percentage (FIG. 9). DMA01-166 showed a dose independent macrophage polarization effect (FIG. 10).

### Example 6

### PI3K Inhibition by Dual Functioning NCEs

PI3Kα, PI3K β, PI3Kγ, and PI3Kδ binding assay kits were obtained from BPS Bioscience (San Diego, CA, USA). The assays were conducted according to manufacturer's protocols in 96-well microplates. Briefly, 5 μL PI3K lipid substrate was added to all wells. Then, 5 μL of IPI-549, DMA01-148, 143, 132, or 166 at different concentrations were added, which were followed by 5 μL of ATP (12.5 μM). 10 μL of PI3Ka (0.5 ng/μL, PI3K β 4 ng/μL, PI3Kγ 4 ng/μL, or PI3Kδ 13 ng/μL) was added to initiate reaction. The plate was carefully shaken, and the reaction mixture was incubated at 30° C. for 40 minutes. Then 25 μL ADP-Glo reagent (Promega, Madison, WI, USA) was added to each well and the reactions were performed in darkness for 45 minutes. Finally, 50 μL of kinase detection reagent (Promega, Madison, WI, USA) was added to each well and the reactions were performed in darkness for 30 minutes. The luminescence of the reaction mixture was read on a Synergy 2 microplate reader (Biotek).

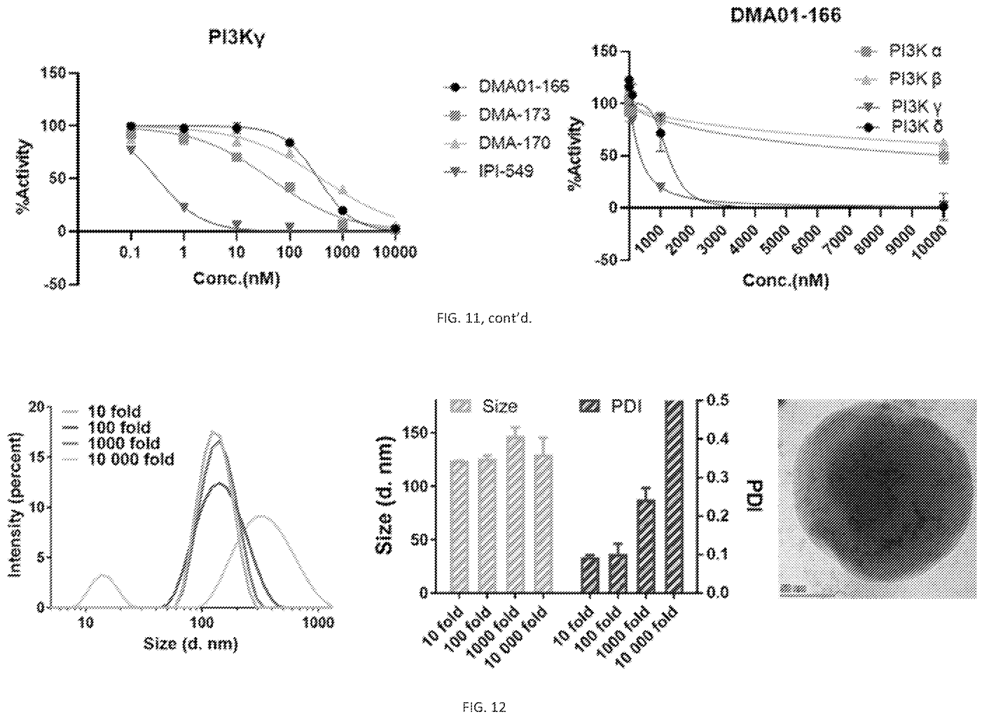

Inhibition of different isoform of PI3Kα, PI3K β, PI3Kγ, and PI3Kδ by IPI-549, DMA01-148, DMA01-143, DMA01-132, DMA01-166, DMA01-170, DMA01-173 were tested (FIG. 11). The IC50 of compounds to different PI3K isoforms were calculated using Prism 8 (Table 1). DMA01-148 selectively inhibited PI3Kγ, with similar efficacy compared to IPI-549. DMA01-143 and DMA01-132 inhibited both PI3Kγ and PI3Kδ. DMA01-166 showed no effect on PI3Kα and PI3K β, but inhibited both PI3Kγ in 349 nM.

TABLE 1
In vitro inhibitory activities (IC50) of IPI-549, DMA01-
148, 143, 132, 166 against different isoforms of PI3Ks

| | Enzyme IC₅₀ (nM) | | | |
|---|---|---|---|---|
| Compounds | PI3Kα | PI3K β | PI3Kγ | PI3Kδ |
| IPI-549 | 186.6 | 106.84 | 0.3620 | 337.7 |
| 148 | 1052 | 547.5 | 0.8636 | 15.44 |
| 143 | 409 | 311.8 | 5.343 | 1.364 |
| 132 | 371.9 | 373.8 | 0.8447 | 0.3811 |
| 166 | >10000 | >10000 | 349.0 | 1250 |

### Example 7

### DMA01-166 Nanoformulation

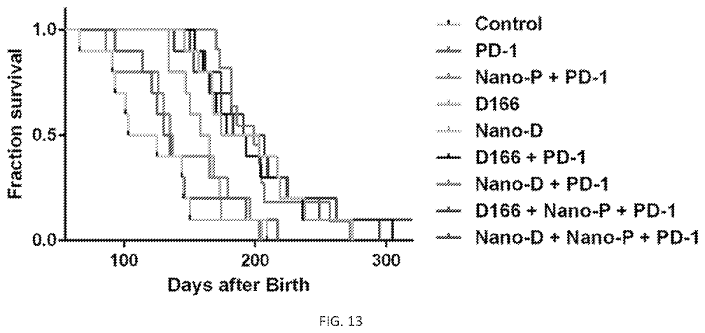

DMA01-166 was dissolved in 1 mL of chloroform (organic phase) and then added dropwise into human serum albumin (66 kDa) dissolved in 20 mL Milli Q water (water phase) to generate a milky emulsion using a rotor-stator homogenizer. The crude Nano-166 emulsion was obtained after running 6 cycles of low pressure (20000 psi) on a high-pressure homogenizer (Nano DeBEE). The organic solvent was removed by rotoevaporation at 25° C. After being filtered by the 0.22 μm strainer, the resulting Nano-P1 suspension was lyophilized to obtain dry powder for long-term storage at −20° C. The size of Nano-166 is around 120 nm with PDI less than 0.2 as tested by Dynamic Light Scattering (DLS). The size measured by TEM is less than 100 nm. The Nano-166 is stable after dilution for 10 000 folds (FIG. 12).

The in vivo efficacy of DMA01-166 and its albumin nanoformulation was tested on KPC transgenic model. DMA01-166 plus PD-1 antibody (D166+PD-1) shows longer median survival of 185.5 days compared to DMA01-166 (D166 157.5 days) or PD-1 alone (PD-1 132.5 days). Nano-D plus PD-1 antibody (Nano-D+PD-1) shows the longest median survival (185.5 days). Adding an albumin nanoformulation of paclitaxel (Nano-P) did not change the median survival time (FIG. 13).

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art and may be made without departing from the spirit and scope thereof.

1. A compound of formula (I): 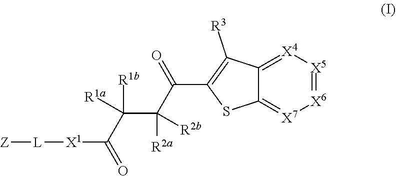
or a pharmaceutically acceptable salt thereof, wherein:
X¹ is selected from O, NR^{w}, S, and a bond;
R^{1a}, R^{1b}, R^{2a}, and R^{2b} are each independently selected from hydrogen, C₁-C₄ alkyl, C₁-C₄ alkoxy, C₃-C₆ cycloalkyl, halo-C₁-C₄-alkyl, amino-C₁-C₄-alkyl, hydroxy-C₁-C₄-alkyl, C₁-C₄-alkoxy-C₁-C₄-alkyl, halo, hydroxy, amino, C₁-C₄-alkylamino, di-C₁-C₄-alkylamino, and cyano; wherein R^{1a} and R^{1b}, or R^{1a} and R^{2a}, or R^{2a} and R^{2b}, are optionally taken together with the carbon atom(s) to which they are attached to form an optionally substituted 3- to 6-membered ring;
X⁴ is CR⁴ or N;
X⁵ is CR⁵ or N;
X⁶ is CR⁶ or N;
X⁷ is CR⁷ or N;
R³, R⁴, R⁵, R⁶, and R⁷ are each independently selected from hydrogen, C₁-C₆ alkyl, C₂-C₆ alkenyl, C₂-C₆ alkynyl, C₁-C₆ alkoxy, C₁-C₆ alkylthio, C₃-C₆ cycloalkyl, halo-C₁-C₆-alkyl, halo-C₁-C₆-alkoxy, amino-C₁-C₆-alkyl, hydroxy-C₁-C₆-alkyl, C₁-C₆-alkoxy-C₁-C₆-alkyl, halo, hydroxy, amino, C₁-C₄-alkylamino, di-C₁-C₄-alkylamino, cyano, —COOR^{x}, —CON(R^{y})₂, —SO₂R², an oligo- or poly-ethylene glycol chain, and a group-Y—R⁸; wherein R⁴ and R⁵, R⁵ and R⁶, or R⁶ and R⁷ are optionally taken together with the carbon atoms to which they are attached to form an optionally substituted 5- or 6-membered ring;
Y is selected from —C(O)—, —C(O)O—, —C(O)NR^{y}—, and —C(O)S—;
R⁸ is a lipid moiety having at least 8 carbon atoms;
R^{v}, R^{w}, R^{x}, R^{y}, and R^{z} are each independently selected from hydrogen, C₁-C₆ alkyl, C₂-C₆ alkenyl, C₂-C₆ alkynyl, C₃-C₆ cycloalkyl, and halo-C₁-C₆-alkyl;
L is a linker; and
Z is a moiety of formula (a) or formula (b): 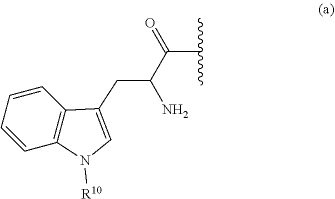
wherein R¹⁰ is hydrogen, or C₁-C₆ alkyl; 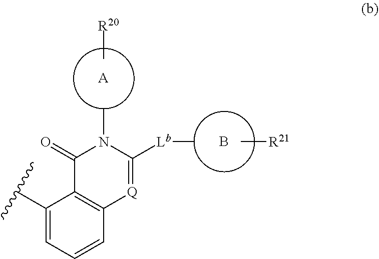
wherein:
Q is CH or N;
A is aryl or a 5- or 6-membered monocyclic heteroaryl having 1, 2, 3, or 4 heteroatoms independently selected from N, O, S, and P;
R²⁰ is selected from hydrogen, halo, C₁-C₄ alkyl, C₃-C₆ cycloalkyl, C₁-C₄ haloalkyl, —OR^{a1}, —N(R^{a2})(R^{a3}), —SO₂R^{a4}, —SO₂N(R^{a5})(R^{a6}), and —NHSO₂R^{a7}, wherein R^{a1}, R^{a2}, R^{a3}, R^{a4}, R^{a5}, R^{a6}, and R^{a7} are each independently selected from hydrogen, C₁-C₄ alkyl, and C₁-C₄ haloalkyl;
R²¹ is selected from hydrogen and a group L²¹-E, wherein:
L²¹ is a bond, C₁-C₂ alkylene, —CH═CH—, —C≡C—, —C(O)—, —O—, —NH—, —S—, —C(O)O—, —C(O)NH—, —C(O)S—, arylene, cycloalkylene, heteroarylene, or heterocyclylene, or wherein L²¹ comprises a combination of any two of such groups;
E is a bicyclic heterocyclyl or bicyclic heteroaryl, each of which is optionally substituted with 1, 2, 3, 4, or 5 substituents independently selected from halo, C₁-C₄ alkyl, C₃-C₆ cycloalkyl, C₃-C₆-cycloalkyl-C₁₋₄-alkyl, C₁-C₄ haloalkyl, oxo, —OR^{b1}, —N(R^{b2})(R^{b3}), —SO₂R^{b4}, —SO₂N(R^{b5})(R^{b6}), and —NHSO₂R^{b7}, wherein R^{b1}, R^{b2}, R^{b3}, R^{b4}, R^{b5}, R^{b6}, and R^{b7} are each independently selected from hydrogen, C₁-C₄ alkyl, and C₁-C₄ haloalkyl;
L^{b} is —(CR^{c1}R^{c2})ₘ-G^{b}-, wherein:
R^{c1} and R^{c2} are independently selected from hydrogen and C₁-C₄ alkyl;
m is 0, 1, or 2; and
G^{b} is a bond, —NHC(O)—, —NH—, —O—, or —S—; and
B is a bicyclic heteroaryl or bicyclic heterocyclyl, each of which is optionally substituted with 1, 2, 3, 4, or 5 substituents independently selected from halo, C₁-C₄ alkyl, C₃-C₆ cycloalkyl, C₁-C₄ haloalkyl, optionally substituted aryl, —OR^{d1}, —N(R^{d2})(R^{d3}), —SO₂R^{d4}, —SO₂N(R^{d5})(R^{d6}), and —NHSO₂R^{d7}, wherein R^{d1}, R^{d2}, R^{d3}, R^{d4}, R^{d5}, R^{d6}, and R^{d7} are each independently selected from hydrogen, C₁-C₄ alkyl, and C₁-C₄ haloalkyl.

2. The compound of claim 1, or a pharmaceutically acceptable salt thereof, wherein X⁴ is CR⁴, X⁵ is CR⁵, X⁶ is CR⁶, and X⁷ is CR⁷.

3. The compound of claim 1 or 2, or a pharmaceutically acceptable salt thereof, wherein R⁴ is selected from hydrogen and halo.

4. The compound of any one of claims 1-3, or a pharmaceutically acceptable salt thereof, wherein R⁴ is hydrogen.

5. The compound of any one of claims 1-4, or a pharmaceutically acceptable salt thereof, wherein R⁵ and R⁶ are each independently selected from C₁-C₆ alkyl, C₂-C₆ alkenyl, C₂-C₆ alkynyl, C₁-C₆ alkoxy, C₁-C₆ alkylthio, C₃-C₆ cycloalkyl, halo-C₁-C₆-alkyl, halo-C₁-C₆-alkoxy, amino-C₁-C₆-alkyl, hydroxy-C₁-C₆-alkyl, C₁-C₆-alkoxy-C₁-C₆-alkyl, halo, hydroxy, amino, C₁-C₄-alkylamino, di-C₁-C₄-alkylamino, cyano, —COOR^{x}, —CON(R^{y})₂, and —SO₂R^{z}.

6. The compound of any one of claims 1-5, or a pharmaceutically acceptable salt thereof, wherein R⁵ and R⁶ are each independently selected from C₁-C₄ alkyl, C₂-C₄ alkenyl, C₂-C₄ alkynyl, C₁-C₄ alkoxy, C₁-C₆ alkylthio, halo-C₁-C₄-alkyl, halo-C₁-C₄-alkoxy, hydroxy, halo, and C₁-C₄-alkylamino.

7. The compound of any one of claims 1-6, or a pharmaceutically acceptable salt thereof, wherein R⁵ and R⁶ are each independently selected from C₁-C₄ alkyl, C₁-C₄ alkoxy, and halo.

8. The compound of any one of claims 1-7, or a pharmaceutically acceptable salt thereof, wherein R⁵ and R⁶ are each independently selected from C₁-C₄ alkoxy.

9. The compound of any one of claims 1-8, or a pharmaceutically acceptable salt thereof, wherein R⁷ is hydrogen.

10. The compound of any one of claims 1-9, or a pharmaceutically acceptable salt thereof, wherein R^{1a}, R^{1b}, R^{2a}, and R^{2b} are each independently selected from hydrogen, C₁-C₄ alkyl, C₃-C₆ cycloalkyl, C₁-C₄-alkoxy-C₁-C₄-alkyl, and hydroxy.

11. The compound of any one of claims 1-10, or a pharmaceutically acceptable salt thereof, wherein R^{1a}, R^{1b}, R^{2a}, and R^{2b} are each independently selected from hydrogen and C₁-C₄ alkyl.

12. The compound of any one of claims 1-11, or a pharmaceutically acceptable salt thereof, wherein R^{1b}, R^{2a}, and R^{2b} are hydrogen, and R^{1a} is selected from hydrogen and C₁-C₄ alkyl.

13. The compound of any one of claims 1-9, or a pharmaceutically acceptable salt thereof, wherein R^{1a} and R^{2a} are hydrogen, and R^{1b} and R^{2b} together with the carbon atoms to which they are attached form a 3-membered ring.

14. The compound of any one of claims 1-13, or a pharmaceutically acceptable salt thereof, wherein R³ is selected from hydrogen and halo.

15. The compound of any one of claims 1-14, or a pharmaceutically acceptable salt thereof, wherein R³ is hydrogen.

16. The compound of any one of claims 1-15, or a pharmaceutically acceptable salt thereof, wherein the compound is a compound of formula (Ia):

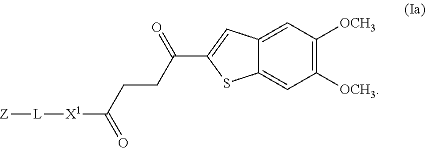

17. The compound of any one of claims 1-16, or a pharmaceutically acceptable salt thereof, wherein Z is a moiety of formula (a).

18. The compounds of claim 17, or a pharmaceutically acceptable salt thereof, wherein R¹⁰ is methyl.

19. The compound of any one of claims 1-16, or a pharmaceutically acceptable salt thereof, wherein Z is a moiety of formula (b).

20. The compound of claim 19, or a pharmaceutically acceptable salt thereof, wherein the moiety of formula (b) is a moiety of formula (bi):

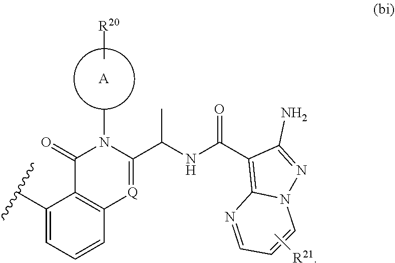

21. The compound of claim 19 or 20, or a pharmaceutically acceptable salt thereof, wherein A is phenyl.

22. The compound of any one of claims 19-21, or a pharmaceutically acceptable salt thereof, wherein R²⁰ is hydrogen.

23. The compound of any one of claims 19-22, or a pharmaceutically acceptable salt thereof, wherein Q is CH.

24. The compound of any one of claims 19-23, or a pharmaceutically acceptable salt thereof, wherein R²¹ is selected from hydrogen and a group of formula:

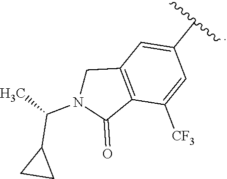 [SMILES: *c1cc2c(c(C(F)(F)F)c1)C(=O)N([C@@H](C)C1CC1)C2]

25. The compound of any one of claims 19-24, or a pharmaceutically acceptable salt thereof, wherein the moiety of formula (b) is:

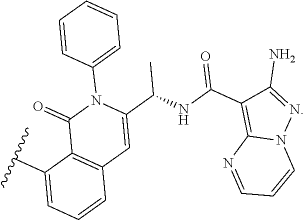 [SMILES: *c1cccc2cc([C@H](C)NC(=O)c3c(N)nn4cccnc34)n(-c3ccccc3)c(=O)c12]

26. The compound of any one of claims 1-25, or a pharmaceutically acceptable salt thereof, wherein L comprises one or more groups independently selected from —C(R′)₂—, —CH═CH—, —C≡C—, —O—, —NR′—, —BR′—, —S—, —C(O)—, —C(NR′)—, —S(O)—, —S(O)₂—, arylene, heteroarylene, cycloalkylene, and heterocyclylene, wherein each R′ is independently selected from hydrogen, C₁-C₈₀ alkyl, C₂-C₈₀ alkenyl, C₂-C₈₀ alkynyl, aryl, arylalkyl, cycloalkyl, cycloalkylalkyl, heterocyclyl, heterocyclylalkyl, heteroaryl, and heteroarylalkyl, and wherein each alkyl, arylene, heteroarylene, cycloalkylene, and heterocyclylene is independently unsubstituted or substituted with 1, 2, or 3 substituents.

27. The compound of claim 26, or a pharmaceutically acceptable salt thereof, wherein L comprises one or more groups independently selected from —C(R′)₂—, —C≡C—, —O—, —NH—, —C(O)—, and heteroarylene, wherein each R′ is independently selected from hydrogen, C₁-C₄₀ alkyl, phenyl, and —CH₂-heterocyclyl (e.g., wherein the heterocyclyl is a 6-membered heterocyclyl having 1 or 2 heteroatoms independently selected from N, O, and S), wherein the phenyl and the heterocyclyl are each independently unsubstituted or substituted with 1 or 2 substituents.

28. The compound of claim 1, wherein the compound is selected from: 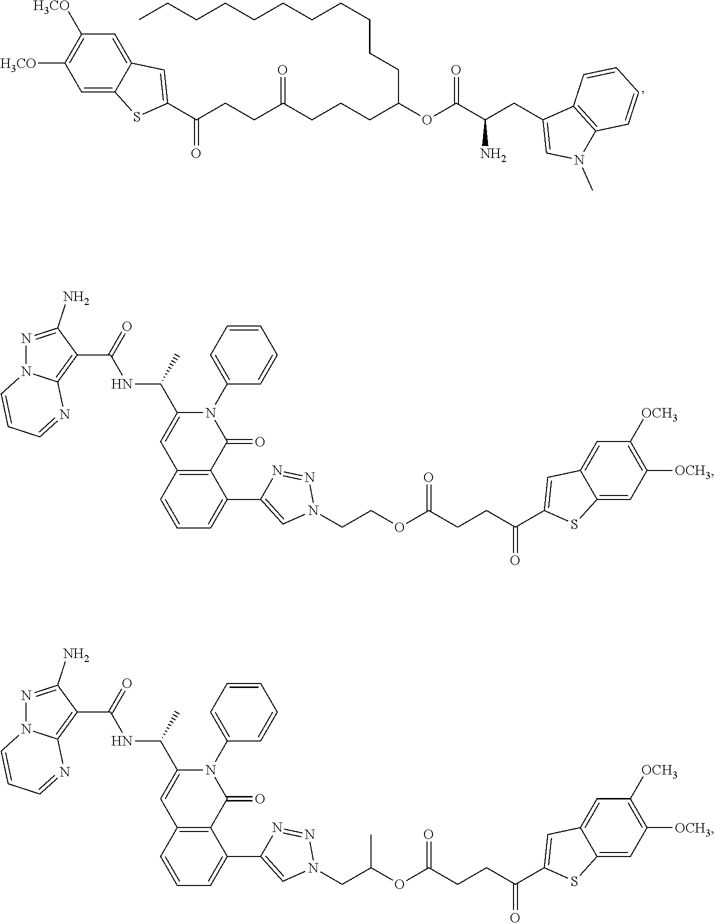 [SMILES: CCCCCCCCCCCC(CCCC(=O)CCC(=O)c1cc2cc(OC)c(OC)cc2s1)OC(=O)[C@H](N)Cc1cn(C)c2ccccc12 ; COc1cc2cc(C(=O)CCC(=O)OCCn3cc(-c4cccc5cc([C@@H](C)NC(=O)c6c(N)nn7cccnc67)n(-c6ccccc6)c(=O)c45)nn3)sc2cc1OC ; COc1cc2cc(C(=O)CCC(=O)OC(C)Cn3cc(-c4cccc5cc([C@@H](C)NC(=O)c6c(N)nn7cccnc67)n(-c6ccccc6)c(=O)c45)nn3)sc2cc1OC]
and pharmaceutically acceptable salts thereof.

29. A pharmaceutical composition comprising an effective amount of a compound of any one of claims 1-28 or a pharmaceutically acceptable salt thereof, and a pharmaceutically acceptable carrier.

30. The pharmaceutical composition of claim 29, wherein the composition comprises albumin nanoparticles, liposomes, micelles, or lipid nanoparticles.

31. The pharmaceutical composition of claim 30, wherein the composition further comprises an albumin nanoparticle.

32. The pharmaceutical composition of claim 31, wherein the albumin is human serum albumin or albumin from animal species.

33. The composition of any of claims 29-32, wherein the composition further comprises at least one additional therapeutic agent.

34. The composition of claim 33, wherein the at least one additional therapeutic agent comprises an immune modulator, a chemotherapeutic agent, a nucleic acid, a decongestant, a steroid, an analgesic, an antimicrobial agent, an immunotherapy, or a combination thereof.

35. The composition of claim 33 or 34, wherein the at least one additional therapeutic agent comprises an RNA selected from the group consisting of a small interfering RNA (siRNA), an asymmetrical interfering RNA (aiRNA), a microRNA (miRNA), a Dicer-substrate RNA (dsRNA), a small hairpin RNA (shRNA), a messenger RNA (mRNA), and mixtures thereof.

36. The composition of claim 33 or 34, wherein the at least one additional therapeutic agent is selected from a chemotherapeutic agent, an IDO inhibitor, a Stat3 inhibitor, a TLR agonist, PD-1 or PD-L¹ antibody, and a PI3K inhibitor.

37. The composition of any of claims 29-36, further comprising one or more cell targeting epitopes.

38. The composition of claim 37, wherein the one or more cell targeting epitopes are covalently attached or directly conjugated to an albumin.

39. The composition of claim 37 or 38, wherein the cell targeting epitopes comprise an immune cell epitope.

40. The composition of claim 37 or 38, further comprising one or more epitopes from a microbiological agent.

41. A vaccine comprising an effective amount of
a compound of any one of claims 1-28, or a pharmaceutically acceptable salt thereof, or a composition of any of one of claims 29-40; and
an antigen or a nucleic acid encoding thereof.

42. The vaccine of claim 41, wherein the antigen is a tumor antigen, a self-antigen, or an infectious disease derived antigen.

43. The vaccine of claim 41 or 42, wherein the nucleic acid is messenger RNA (mRNA).

44. A method of treating or preventing a disease or disorder comprising administering an effective amount of a compound of any one of claims 1-28, or a pharmaceutically acceptable salt thereof, a composition of any of claims 29-40, or the vaccine of any of claims 41-43, to a subject in need thereof.

45. The method of claim 44, wherein the disease or disorder comprises cancer, an autoimmune disease, an inflammatory disease, or an infectious disease.

46. The method of claim 44 or 45, wherein the disease or disorder is cancer.

47. The method of claim 46, wherein the subject has cancer, has had cancer, is predisposed to cancer, or has a family history of cancer.

48. The method of any of claims 45-47, wherein the cancer comprises a solid tumor or hematological cancer.

49. The method of any of claims 45-48, wherein the cancer is metastatic cancer.

50. The method of any of claims 45-49, wherein the method suppresses or eliminates cancer metastasis, decreases tumor growth, prevents tumor recurrences, or any combination thereof.

51. The method of any of claims 44-50, wherein the administering comprises an initial immunization and at least one subsequent immunization.

52. A method of inducing or modulating an immune or inflammatory response in a subject comprising administering a compound of any one of claims 1-28 or a pharmaceutically acceptable salt thereof, a composition of any of claims 29-40, or the vaccine of any of claims 41-43, to a subject in need thereof.

53. The method of any of claims 44-52, wherein the subject is human.

54. The method of any of claims 44-53, further comprising administering at least one additional therapeutic agent.

55. The method of claim 54, wherein the at least one additional therapeutic agent comprises an immune modulator, a chemotherapeutic agent, a nucleic acid, a decongestant, a steroid, an analgesic, an antimicrobial agent, an immunotherapy, or a combination thereof.

56. Use of a compound of any one of claims 1-28, or a pharmaceutically acceptable salt thereof, or a composition of any of claims 29-40 in the manufacture of a medicament for the treatment or prevention a disease or disorder.

57. The use of claim 56, wherein the disease or disorder comprises cancer, an autoimmune disease, an inflammatory disease, or an infectious disease.
